SEQUENCE LISTING (139 sequences)

SEQ ID NO: 1 - protein, 442 aa, Artificial Sequence
  Synthetic Construct
EVQLLESGGGLVQPGGSLRLSCAASGRTFSSYAMGWFRQAPGKEREFVSAISGSGGSTYYADSVKGRFTISRDNSKNTLYLQMNSLKPEDTAVYYCAADLGDGSWVDYVNMEPYEYDYWGQGTQVTVSSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKGSHHHHHH

SEQ ID NO: 2 - protein, 476 aa, Artificial Sequence
  Synthetic Construct
CRGDRGDRGDRGDCGGGGAEVQLLESGGGLVQPGGSLRLSCAASGRTFSSYAMGWFRQAPGKEREFVSAISGSGGSTYYADSVKGRFTISRDNSKNTLYLQMNSLKPEDTAVYYCAADLGDGSWVDYVNMEPYEYDYWGQGTQVTVSSGGGGAGGGGAGGGGAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKGSHHHHHH

SEQ ID NO: 3 - protein, 468 aa, Artificial Sequence
  Synthetic Construct
CRGDRGDRGDRGDCGGGGAEVQLLESGGGLVQPGGSLRLSCAASGRTFSSYAMGWFRQAPGKEREFVSAISGSGGSTYYADSVKGRFTISRDNSKNTLYLQMNSLKPEDTAVYYCAADLGDGSWVDYVNMEPYEYDYWGQGTQVTVSSGGGGAGGGGAGGGGAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 4 - protein, 324 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKGGGGACRGDRGDRGDRGDC

SEQ ID NO: 5 - protein, 453 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKQVQLVESGGGLVKPGGSLRLSCAASGRTFSSYAMGWFRQAPGKEREFVSAISGSGGSTYYADSVKGRFTISRDNAKNSLYLQMNSLRAEDTAVYYCAADLGDGSWVDYVNAEPYEYDYWGQGTLVTVSSGGGGACRGDRGDRGDRGDC

SEQ ID NO: 6 - protein, 442 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLGGGGAGGGGAGGGGAAYCGPPPSINNGDTTSFPLSVYPPGSTVTYRCQSFYKLQGSVTVTCRNKQWSEPPRCLDPCVVSEENMNKNNIQLKWRNDGKLYAKTGDAVEFQCKFPHKAMISSPPFRAICQEGKFEYPICE

SEQ ID NO: 7 - protein, 434 aa, Artificial Sequence
  Synthetic Construct
QVQLVESGGGLVKPGGSLRLSCAASGRTFSSYAMGWFRQAPGKEREFVSAISGSGGSTYYADSVKGRFTISRDNAKNSLYLQMNSLRAEDTAVYYCAADLGDGSWVDYVNAEPYEYDYWGQGTLVTVSSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 8 - protein, 324 aa, Artificial Sequence
  Synthetic Construct
QDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKGGGGACRGDRGDRGDRGDC

SEQ ID NO: 9 - protein, 453 aa, Artificial Sequence
  Synthetic Construct
QDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKQVQLVESGGGLVKPGGSLRLSCAASGRTFSSYAMGWFRQAPGKEREFVSAISGSGGSTYYADSVKGRFTISRDNAKNSLYLQMNSLRAEDTAVYYCAADLGDGSWVDYVNAEPYEYDYWGQGTLVTVSSGGGGACRGDRGDRGDRGDC

SEQ ID NO: 10 - protein, 446 aa, Artificial Sequence
  Synthetic Construct
QDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLGGGGAGGGGAGGGAGGGGSAYCGPPPSINNGDTTSFPLSVYPPGSTVTYRCQSFYKLQGSVTVTCRNKQWSEPPRCLDPCVVSEENMNKNNIQLKWRNDGKLYAKTGDAVEFQCKFPHKAMISSPPFRAICQEGKFEYPICE

SEQ ID NO: 11 - protein, 434 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKQVQLVESGGGLVKPGGSLRLSCAASGRTFSSYAMGWFRQAPGKEREFVSAISGSGGSTYYADSVKGRFTISRDNAKNSLYLQMNSLRAEDTAVYYCAADLGDGSWVDYVNAEPYEYDYWGQGTLVTVSS

SEQ ID NO: 12 - protein, 434 aa, Artificial Sequence
  Synthetic Construct
QDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKQVQLVESGGGLVKPGGSLRLSCAASGRTFSSYAMGWFRQAPGKEREFVSAISGSGGSTYYADSVKGRFTISRDNAKNSLYLQMNSLRAEDTAVYYCAADLGDGSWVDYVNAEPYEYDYWGQGTLVTVSS

SEQ ID NO: 13 - protein, 453 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKEVQLVESGGGLVKPGGSLRLSCAASGRTFSSYAMGWFRQAPGKEREFVSAISGSGGSTYYADSVKGRFTISRDNAKNSLYLQMNSLRAEDTAVYYCAADLGDGSWVDYVNAEPYEYDYWGQGTLVTVSSGGGGACRGDRGDRGDRGDC

SEQ ID NO: 14 - protein, 516 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLEVQLVESGGGLVKPGGSLRLSCAASGRTFSSYAMGWFRQAPGKEREFVSAISGSGGSTYYADSVKGRFTISRDNAKNSLYLQMNSLRAEDTAVYYCAADLGDGSWVDYVNAEPYEYDYWGQGTLVTVSSGGGGACRGDRGDRGDRGDC

SEQ ID NO: 15 - protein, 458 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKGGGGAEVQLVESGGGLVKPGGSLRLSCAASGRTFSSYAMGWFRQAPGKEREFVSAISGSGGSTYYADSVKGRFTISRDNAKNSLYLQMNSLRAEDTAVYYCAADLGDGSWVDYVNAEPYEYDYWGQGTLVTVSSGGGGACRGDRGDRGDRGDC

SEQ ID NO: 16 - protein, 305 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 17 - protein, 305 aa, Artificial Sequence
  Synthetic Construct
QDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 18 - protein, 368 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCL

SEQ ID NO: 19 - protein, 129 aa, Artificial Sequence
  Synthetic Construct
QVQLVESGGGLVKPGGSLRLSCAASGRTFSSYAMGWFRQAPGKEREFVSAISGSGGSTYYADSVKGRFTISRDNAKNSLYLQMNSLRAEDTAVYYCAADLGDGSWVDYVNAEPYEYDYWGQGTLVTVSS

SEQ ID NO: 20 - protein, 129 aa, Artificial Sequence
  Synthetic Construct
EVQLVESGGGLVKPGGSLRLSCAASGRTFSSYAMGWFRQAPGKEREFVSAISGSGGSTYYADSVKGRFTISRDNAKNSLYLQMNSLRAEDTAVYYCAADLGDGSWVDYVNAEPYEYDYWGQGTLVTVSS

SEQ ID NO: 21 - protein, 14 aa, Artificial Sequence
  Synthetic Construct
CRGDRGDRGDRGDC

SEQ ID NO: 22 - protein, 123 aa, Artificial Sequence
  Synthetic Construct
AYCGPPPSINNGDTTSFPLSVYPPGSTVTYRCQSFYKLQGSVTVTCRNKQWSEPPRCLDPCVVSEENMNKNNIQLKWRNDGKLYAKTGDAVEFQCKFPHKAMISSPPFRAICQEGKFEYPICE

SEQ ID NO: 23 - protein, 129 aa, Artificial Sequence
  Synthetic Construct
EVQLLESGGGLVQPGGSLRLSCAASGRTFSSYAMGWFRQAPGKEREFVSAISGSGGSTYYADSVKGRFTISRDNSKNTLYLQMNSLKPEDTAVYYCAADLGDGSWVDYVNMEPYEYDYWGQGTQVTVSS

SEQ ID NO: 24 - protein, 64 aa, Artificial Sequence
  Synthetic Construct
EDCNELPPRRNTEILTGSWSDQTYPEGTQAIYKCRPGYRSLGNVIMVCRKGEWVALNPLRKCQK

SEQ ID NO: 25 - protein, 61 aa, Artificial Sequence
  Synthetic Construct
RPCGHPGDTPFGTFTLTGGNVFEYGVKAVYTCNEGYQLLGEINYRECDTDGWTNDIPICEV

SEQ ID NO: 26 - protein, 64 aa, Artificial Sequence
  Synthetic Construct
VKCLPVTAPENGKIVSSAMEPDREYHFGQAVRFVCNSGYKIEGDEEMHCSDDGFWSKEKPKCVE

SEQ ID NO: 27 - protein, 57 aa, Artificial Sequence
  Synthetic Construct
ISCKSPDVINGSPISQKIIYKENERFQYKCNMGYEYSERGDAVCTESGWRPLPSCEE

SEQ ID NO: 28 - protein, 59 aa, Artificial Sequence
  Synthetic Construct
KSCDNPYIPNGDYSPLRIKHRTGDEITYQCRNGFYPATRGNTAKCTSTGWIPAPRCTLK

SEQ ID NO: 29 - protein, 63 aa, Artificial Sequence
  Synthetic Construct
PCDYPDIKHGGLYHENMRRPYFPVAVGKYYSYYCDEHFETPSGSYWDHIHCTQDGWSPAVPCL

SEQ ID NO: 30 - protein, 19 aa, Artificial Sequence
  Synthetic Construct
GGGGAGGGGAGGGAGGGGS

SEQ ID NO: 31 - protein, 8 aa, Artificial Sequence
  Synthetic Construct
GGGGSDAA

SEQ ID NO: 32 - protein, 15 aa, Artificial Sequence
  Synthetic Construct
GGGGAGGGGAGGGGS

SEQ ID NO: 33 - protein, 14 aa, Artificial Sequence
  Synthetic Construct
GGGGAGGGAGGGGS

SEQ ID NO: 34 - protein, 15 aa, Artificial Sequence
  Synthetic Construct
GGGGAGGGGAGGGGS

SEQ ID NO: 35 - protein, 15 aa, Artificial Sequence
  Synthetic Construct
GGGGSGGGGSGGGGS

SEQ ID NO: 36 - protein, 5 aa, Artificial Sequence
  Synthetic Construct
GGGGS

SEQ ID NO: 37 - protein, 10 aa, Artificial Sequence
  Synthetic Construct
GGGGSGGGGS

SEQ ID NO: 38 - protein, 15 aa, Artificial Sequence
  Synthetic Construct
GGGGSGGGGSGGGGS

SEQ ID NO: 39 - protein, 20 aa, Artificial Sequence
  Synthetic Construct
GGGGSGGGGSGGGGSGGGGS

SEQ ID NO: 40 - protein, 25 aa, Artificial Sequence
  Synthetic Construct
GGGGSGGGGSGGGGSGGGGSGGGGS

SEQ ID NO: 41 - protein, 30 aa, Artificial Sequence
  Synthetic Construct
GGGGSGGGGSGGGGSGGGGSGGGGSGGGGS

SEQ ID NO: 42 - protein, 15 aa, Artificial Sequence
  Synthetic Construct
EAAAKEAAAKEAAAK

SEQ ID NO: 43 - protein, 5 aa, Artificial Sequence
  Synthetic Construct
PAPAP

SEQ ID NO: 44 - protein, 10 aa, Artificial Sequence
  Synthetic Construct
GGGGSPAPAP

SEQ ID NO: 45 - protein, 10 aa, Artificial Sequence
  Synthetic Construct
PAPAPGGGGS

SEQ ID NO: 46 - protein, 12 aa, Artificial Sequence
  Synthetic Construct
GSTSGKSSEGKG

SEQ ID NO: 47 - protein, 10 aa, Artificial Sequence
  Synthetic Construct
GGGDSGGGDS

SEQ ID NO: 48 - protein, 10 aa, Artificial Sequence
  Synthetic Construct
GGGESGGGES

SEQ ID NO: 49 - protein, 10 aa, Artificial Sequence
  Synthetic Construct
GGGDSGGGGS

SEQ ID NO: 50 - protein, 10 aa, Artificial Sequence
  Synthetic Construct
GGGASGGGGS

SEQ ID NO: 51 - protein, 10 aa, Artificial Sequence
  Synthetic Construct
GGGESGGGGS

SEQ ID NO: 52 - protein, 6 aa, Artificial Sequence
  Synthetic Construct
ASTKGP

SEQ ID NO: 53 - protein, 13 aa, Artificial Sequence
  Synthetic Construct
ASTKGPSVFPLAP

SEQ ID NO: 54 - protein, 4 aa, Artificial Sequence
  Synthetic Construct
GGGP

SEQ ID NO: 55 - protein, 8 aa, Artificial Sequence
  Synthetic Construct
GGGGGGGP

SEQ ID NO: 56 - protein, 9 aa, Artificial Sequence
  Synthetic Construct
PAPNLLGGP

SEQ ID NO: 57 - protein, 6 aa, Artificial Sequence
  Synthetic Construct
GGGGGG

SEQ ID NO: 58 - protein, 12 aa, Artificial Sequence
  Synthetic Construct
GGGGGGGGGGGG

SEQ ID NO: 59 - protein, 8 aa, Artificial Sequence
  Synthetic Construct
APELPGGP

SEQ ID NO: 60 - protein, 8 aa, Artificial Sequence
  Synthetic Construct
SEPQPQPG

SEQ ID NO: 61 - protein, 12 aa, Artificial Sequence
  Synthetic Construct
GGGSGGGSGGGS

SEQ ID NO: 62 - protein, 14 aa, Artificial Sequence
  Synthetic Construct
GGGGGGGGGSGGGS

SEQ ID NO: 63 - protein, 15 aa, Artificial Sequence
  Synthetic Construct
GGGGSGGGGGGGGGS

SEQ ID NO: 64 - protein, 15 aa, Artificial Sequence
  Synthetic Construct
GGSSSGGSSSGGSSS

SEQ ID NO: 65 - protein, 20 aa, Artificial Sequence
  Synthetic Construct
GSSSSGSSSSGSSSSGSSSS

SEQ ID NO: 66 - protein, 15 aa, Artificial Sequence
  Synthetic Construct
GGGGAGGGGSGGGGS

SEQ ID NO: 67 - protein, 15 aa, Artificial Sequence
  Synthetic Construct
GGGGSGGGGAGGGGS

SEQ ID NO: 68 - protein, 15 aa, Artificial Sequence
  Synthetic Construct
GGGASGGGGSGGGGS

SEQ ID NO: 69 - protein, 15 aa, Artificial Sequence
  Synthetic Construct
GGGGSGGGASGGGGS

SEQ ID NO: 70 - protein, 15 aa, Artificial Sequence
  Synthetic Construct
GGGGSAGGGSGGGGS

SEQ ID NO: 71 - protein, 15 aa, Artificial Sequence
  Synthetic Construct
GGGGSGGGGSAGGGS

SEQ ID NO: 72 - protein, 15 aa, Artificial Sequence
  Synthetic Construct
GGGGSAGGGSAGGGS

SEQ ID NO: 73 - protein, 15 aa, Artificial Sequence
  Synthetic Construct
GGGGDGGGGSGGGGS

SEQ ID NO: 74 - protein, 15 aa, Artificial Sequence
  Synthetic Construct
GGGGSGGGGDGGGGS

SEQ ID NO: 75 - protein, 15 aa, Artificial Sequence
  Synthetic Construct
GGGGDGGGGDGGGGS

SEQ ID NO: 76 - protein, 15 aa, Artificial Sequence
  Synthetic Construct
GGGGEGGGGSGGGGS

SEQ ID NO: 77 - protein, 16 aa, Artificial Sequence
  Synthetic Construct
GGGGSGGGGSEGGGGS

SEQ ID NO: 78 - protein, 15 aa, Artificial Sequence
  Synthetic Construct
GGGGEGGGGEGGGGS

SEQ ID NO: 79 - protein, 7 aa, Artificial Sequence
  Synthetic Construct
GGGGSDA

SEQ ID NO: 80 - protein, 5 aa, Artificial Sequence
  Synthetic Construct
GGGGA

SEQ ID NO: 81 - protein, 15 aa, Artificial Sequence
  Synthetic Construct
GGGGAGGGGAGGGGA

SEQ ID NO: 82 - protein, 18 aa, Artificial Sequence
  Synthetic Construct
KESGSVSSEQLAQFRSLD

SEQ ID NO: 83 - protein, 14 aa, Artificial Sequence
  Synthetic Construct
EGKSSGSGSESKST

SEQ ID NO: 84 - protein, 8 aa, Artificial Sequence
  Synthetic Construct
GGGGGGGG

SEQ ID NO: 85 - protein, 12 aa, Artificial Sequence
  Synthetic Construct
GSAGSAAGSGEF

SEQ ID NO: 86 - protein, 7 aa, Artificial Sequence
  Synthetic Construct
AEAAAKA

SEQ ID NO: 87 - protein, 20 aa, Artificial Sequence
  Synthetic Construct
LEAGCKNFFPRSFTSCGSLE

SEQ ID NO: 88 - protein, 4 aa, Artificial Sequence
  Synthetic Construct
GSST

SEQ ID NO: 89 - protein, 12 aa, Artificial Sequence
  Synthetic Construct
CRRRRRREAEAC

SEQ ID NO: 90 - protein, 4 aa, Artificial Sequence
  Synthetic Construct
GSGS

SEQ ID NO: 91 - protein, 6 aa, Artificial Sequence
  Synthetic Construct
GSGSGS

SEQ ID NO: 92 - protein, 8 aa, Artificial Sequence
  Synthetic Construct
GSGSGSGS

SEQ ID NO: 93 - protein, 10 aa, Artificial Sequence
  Synthetic Construct
GSGSGSGSGS

SEQ ID NO: 94 - protein, 5 aa, Artificial Sequence
  Synthetic Construct
EAAAK

SEQ ID NO: 95 - protein, 12 aa, Artificial Sequence
  Synthetic Construct
GSGSGSGSGSGS

SEQ ID NO: 96 - protein, 6 aa, Artificial Sequence
  Synthetic Construct
GGSGGS

SEQ ID NO: 97 - protein, 9 aa, Artificial Sequence
  Synthetic Construct
GGSGGSGGS

SEQ ID NO: 98 - protein, 12 aa, Artificial Sequence
  Synthetic Construct
GGSGGSGGSGGS

SEQ ID NO: 99 - protein, 4 aa, Artificial Sequence
  Synthetic Construct
GGSG

SEQ ID NO: 100 - protein, 8 aa, Artificial Sequence
  Synthetic Construct
GGSGGGSG

SEQ ID NO: 101 - protein, 12 aa, Artificial Sequence
  Synthetic Construct
GGSGGGSGGGSG

SEQ ID NO: 102 - protein, 10 aa, Artificial Sequence
  Synthetic Construct
GENLYFQSGG

SEQ ID NO: 103 - protein, 8 aa, Artificial Sequence
  Synthetic Construct
SACYCELS

SEQ ID NO: 104 - protein, 5 aa, Artificial Sequence
  Synthetic Construct
RSIAT

SEQ ID NO: 105 - protein, 17 aa, Artificial Sequence
  Synthetic Construct
RPACKIPNDLKQKVMNH

SEQ ID NO: 106 - protein, 36 aa, Artificial Sequence
  Synthetic Construct
GGSAGGSGSGSSGGSSGASGTGTAGGTGSGSGTGSG

SEQ ID NO: 107 - protein, 17 aa, Artificial Sequence
  Synthetic Construct
AAANSSIDLISVPVDSR

SEQ ID NO: 108 - protein, 36 aa, Artificial Sequence
  Synthetic Construct
GGSGGGSEGGGSEGGGSEGGGSEGGGSEGGGSGGGS

SEQ ID NO: 109 - protein, 15 aa, Artificial Sequence
  Synthetic Construct
GGGGAGGGGAGGGGS

SEQ ID NO: 110 - protein, 20 aa, Artificial Sequence
  Synthetic Construct
GGGGAGGGGAGGGGAGGGGS

SEQ ID NO: 111 - protein, 28 aa, Artificial Sequence
  Synthetic Construct
DAAGGGGSGGGGSGGGGSGGGGSGGGGS

SEQ ID NO: 112 - protein, 18 aa, Artificial Sequence
  Synthetic Construct
GGSSRSSSSGGGGAGGGG

SEQ ID NO: 113 - protein, 21 aa, Artificial Sequence
  Synthetic Construct
KGGGGAGGGGAGGGAGGGGSK

SEQ ID NO: 114 - protein, 21 aa, Artificial Sequence
  Synthetic Construct
RGGGGAGGGGAGGGAGGGGSR

SEQ ID NO: 115 - protein, 21 aa, Artificial Sequence
  Synthetic Construct
KGGGGAGGGGAGGGAGGGGSR

SEQ ID NO: 116 - protein, 21 aa, Artificial Sequence
  Synthetic Construct
KGGGGAGGGGAGGGAGGGGSR

SEQ ID NO: 117 - protein, 17 aa, Artificial Sequence
  Synthetic Construct
KGGGGAGGGGAGGGGSK

SEQ ID NO: 118 - protein, 17 aa, Artificial Sequence
  Synthetic Construct
KGGGGAGGGGAGGGGSR

SEQ ID NO: 119 - protein, 17 aa, Artificial Sequence
  Synthetic Construct
RGGGGAGGGGAGGGGSK

SEQ ID NO: 120 - protein, 17 aa, Artificial Sequence
  Synthetic Construct
RGGGGAGGGGAGGGGSR

SEQ ID NO: 121 - protein, 5 aa, Artificial Sequence
  Synthetic Construct
DDDDK

SEQ ID NO: 122 - protein, 8 aa, Artificial Sequence
  Synthetic Construct
LEVLFQGP

SEQ ID NO: 123 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 124 - protein, 569 aa, Homo sapiens
MLLLFSVILISWVSTVGGEGTLCDFPKIHHGFLYDEEDYNPFSQVPTGEVFYYSCEYNFVSPSKSFWTRITCTEEGWSPTPKCLRMCSFPFVKNGHSESSGLIHLEGDTVQIICNTGYSLQNNEKNISCVERGWSTPPICSFTKGECHVPILEANVDAQPKKESYKVGDVLKFSCRKNLIRVGSDSVQCYQFGWSPNFPTCKGQVRSCGPPPQLSNGEVKEIRKEEYGHNEVVEYDCNPNFIINGPKKIQCVDGEWTTLPTCVEQVKTCGYIPELEYGYVQPSVPPYQHGVSVEVNCRNEYAMIGNNMITCINGIWTELPMCVATHQLKRCKIAGVNIKTLLKLSGKEFNHNSRIRYRCSDIFRYRHSVCINGKWNPEVDCTEKREQFCPPPPQIPNAQNMTTTVNYQDGEKVAVLCKENYLLPEAKEIVCKDGRWQSLPRCVESTAYCGPPPSINNGDTTSFPLSVYPPGSTVTYRCQSFYKLQGSVTVTCRNKQWSEPPRCLDPCVVSEENMNKNNIQLKWRNDGKLYAKTGDAVEFQCKFPHKAMISSPPFRAICQEGKFEYPICE

SEQ ID NO: 125 - DNA, 1326 bp, Artificial Sequence
  Synthetic Construct
gaggtgcaactgctggaatctggcggaggacttgttcagcctggcggctctctgagactgtcttgtgccgccagcggcagaacctttagctcttacgccatgggctggttcagacaggcccctggcaaagagagagagttcgttagcgccatcagcggctctggcggcagcacatattacgccgatagcgtgaagggcagattcaccatcagccgggacaacagcaagaacaccctgtacctgcagatgaacagcctgaagcctgaggacaccgccgtgtattactgtgccgccgatctcggagatggcagctgggtcgactacgtgaacatggaaccctacgagtacgactactggggccagggcacacaagtgaccgtgtcctctgaggactgcaacgagctgcctcctcggagaaataccgagatcctgaccggctcttggagcgaccagacataccctgagggaacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcaccctgacaggcggcaacgtgttcgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacctgacagagagtatcacttcggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtatagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccccgccaccagaggcaataccgccaagtgtacaagcaccggctggatccctgctcctagatgtacactgaagggctcccaccaccaccatcaccac

SEQ ID NO: 126 - DNA, 1428 bp, Artificial Sequence
  Synthetic Construct
tgccggggagacagaggcgatagaggcgacaggggagattgcggcggaggcggagctgaagttcaactgcttgaatctggcggcggactggttcaacctggcggatctctgagactgtcttgtgccgccagcggcagaacctttagctcttacgccatgggctggttcagacaggcccctggcaaagagagagagttcgttagcgccatctctggcagcggcggcagcacatattacgccgattctgtgaagggcagattcaccatcagccgggacaacagcaagaacaccctgtacctgcagatgaacagcctgaagcctgaggacaccgccgtgtattactgtgccgccgatctcggagatggcagctgggtcgactacgtgaacatggaaccctacgagtacgactactggggccagggcacacaagtgacagtttctagcggtggtggcggagctggtggcggtggtgctggtggcggaggcgctgaagattgcaatgagctgcctcctcggcggaacacagagatcttgacaggctcttggagcgaccagacataccctgagggaacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcaccctgaccggcggcaatgtgtttgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacctgacagagagtatcacttcggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtatagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccctgccaccagaggcaacaccgccaagtgtacaagcacaggctggatccctgctcctagatgtaccctgaagggctcccaccaccaccatcaccat

SEQ ID NO: 127 - DNA, 1404 bp, Artificial Sequence
  Synthetic Construct
tgccggggagacagaggcgatagaggcgacaggggagattgcggcggaggcggagctgaagttcaactgcttgaatctggcggcggactggttcaacctggcggatctctgagactgtcttgtgccgccagcggcagaacctttagctcttacgccatgggctggttcagacaggcccctggcaaagagagagagttcgttagcgccatctctggcagcggcggcagcacatattacgccgattctgtgaagggcagattcaccatcagccgggacaacagcaagaacaccctgtacctgcagatgaacagcctgaagcctgaggacaccgccgtgtattactgtgccgccgatctcggagatggcagctgggtcgactacgtgaacatggaaccctacgagtacgactactggggccagggcacacaagtgacagtttctagcggtggtggcggagctggtggcggtggtgctggtggcggaggcgctgaagattgcaatgagctgcctcctcggcggaacacagagatcttgacaggctcttggagcgaccagacataccctgagggaacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcaccctgaccggcggcaatgtgtttgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacctgacagagagtatcacttcggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtatagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccctgccaccagaggcaacaccgccaagtgtacaagcacaggctggatccctgctcctcggtgcaccctgaaa

SEQ ID NO: 128 - DNA, 972 bp, Artificial Sequence
  Synthetic Construct
gaggattgcaatgagctgcctcctcggagaaacaccgagatcctgacaggctcttggagcgaccagacataccctgagggcacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcacactgaccggcggcaacgtgttcgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtacagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccccgccaccagaggcaataccgccaagtgtacaagcaccggctggatccctgctcctagatgtacacttaaaggcggcggaggcgcctgtaggggagacagaggcgatagaggcgacaggggagattgc

SEQ ID NO: 129 - DNA, 1359 bp, Artificial Sequence
  Synthetic Construct
gaggattgcaatgagctgcctcctcggagaaacaccgagatcctgacaggctcttggagcgaccagacataccctgagggcacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcacactgaccggcggcaacgtgttcgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtacagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccccgccaccagaggcaataccgccaagtgtacaagcaccggctggatccctgctcctagatgcacactgaaacaggtgcagctggtggaatctggcggcggacttgtgaaacctggcggctctctgagactgtcttgtgccgccagcggcagaacctttagcagctacgccatgggatggttcagacaggcccctggcaaagagagagagttcgttagcgccatcagcggctctggcggcagcacatattacgccgatagcgtgaagggcagattcaccatcagccgggacaacgccaagaacagcctgtacctgcagatgaactccctgagagccgaggacaccgccgtgtactattgtgccgctgatctcggagatggcagctgggtcgactatgtgaatgccgagccttacgagtacgactactggggccagggcacactggttacagttagttctggcggaggcggagcctgtaggggagacagaggcgatagaggcgacaggggagattgc

SEQ ID NO: 130 - DNA, 1326 bp, Artificial Sequence
  Synthetic Construct
gaggactgcaacgagctgcctcctagaagaaacaccgagatcctgaccggctcttggagcgaccagacatatcctgagggcacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcaccctgacaggcggcaacgtgttcgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtacagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccccgccaccagaggcaataccgccaagtgtacaagcaccggctggatccctgctcctagatgtactcttggaggtggcggagctggcggcggaggtgctggcggaggcggagcagcttattgtggacctcctcctagcatcaacaacggggacaccaccagctttccactgtctgtgtacccacctggcagcaccgtgacatacagatgccagagcttttacaagctgcagggcagcgtgaccgtgacctgcagaaacaaacagtggtccgagcctccaagatgtctggacccttgtgtggtgtccgaggaaaacatgaacaagaacaacatccagctgaagtggcggaacgacggcaagctgtatgccaagactggggacgccgtggaatttcagtgcaagttccctcacaaggccatgatcagcagccctccattcagagccatctgccaagagggcaagttcgagtaccccatctgcgag

SEQ ID NO: 131 - DNA, 1302 bp, Artificial Sequence
  Synthetic Construct
caggtgcaactggttgaatctggcggcggacttgtgaagcctggcggatctctgagactgtcttgtgccgccagcggcagaacctttagcagctacgccatgggatggttcagacaggcccctggcaaagagagagagttcgttagcgccatctctggcagcggcggcagcacatattacgccgattctgtgaagggcagattcaccatcagccgggacaacgccaagaacagcctgtacctgcagatgaactccctgagagccgaggacaccgccgtgtactattgtgccgctgatctcggagatggcagctgggtcgactatgtgaatgccgagccttacgagtacgactactggggccagggcacactggttaccgtgtcctctgaggactgcaacgagctgcctcctcggagaaataccgagatcctgaccggctcttggagcgaccagacatatcctgagggcacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcaccctgacaggcggcaacgtgttcgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtacagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccccgccaccagaggcaataccgccaagtgtacaagcaccggctggatccctgctcctcggtgcacactgaaa

SEQ ID NO: 132 - DNA, 972 bp, Artificial Sequence
  Synthetic Construct
caggattgcaatgagctgcctcctcggagaaacaccgagatcctgacaggctcttggagcgaccagacataccctgagggcacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcacactgaccggcggcaacgtgttcgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtacagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccccgccaccagaggcaataccgccaagtgtacaagcaccggctggatccctgctcctagatgtacacttaaaggcggcggaggcgcctgtaggggagacagaggcgatagaggcgacaggggagattgc

SEQ ID NO: 133 - DNA, 1359 bp, Artificial Sequence
  Synthetic Construct
caggattgcaatgagctgcctcctcggagaaacaccgagatcctgacaggctcttggagcgaccagacataccctgagggcacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcacactgaccggcggcaacgtgttcgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtacagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccccgccaccagaggcaataccgccaagtgtacaagcaccggctggatccctgctcctagatgcacactgaaacaggtgcagctggtggaatctggcggcggacttgtgaaacctggcggctctctgagactgtcttgtgccgccagcggcagaacctttagcagctacgccatgggatggttcagacaggcccctggcaaagagagagagttcgttagcgccatcagcggctctggcggcagcacatattacgccgatagcgtgaagggcagattcaccatcagccgggacaacgccaagaacagcctgtacctgcagatgaactccctgagagccgaggacaccgccgtgtactattgtgccgctgatctcggagatggcagctgggtcgactatgtgaatgccgagccttacgagtacgactactggggccagggcacactggttacagttagttctggcggaggcggagcctgtaggggagacagaggcgatagaggcgacaggggagattgc

SEQ ID NO: 134 - DNA, 1338 bp, Artificial Sequence
  Synthetic Construct
caggactgcaacgagctgcctcctagaagaaacaccgagatcctgaccggctcttggagcgaccagacatatcctgagggcacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcaccctgacaggcggcaacgtgttcgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtacagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccccgccaccagaggcaataccgccaagtgtacaagcaccggctggatccctgctcctagatgtactcttggaggtggcggagctggcggcggaggtgctggtggtggtgctggcggaggcggatctgcttattgtggacctcctcctagcatcaacaacggggacaccacaagcttcccactgtctgtgtacccacctggcagcaccgtgacatacagatgccagagcttttacaagctgcagggcagcgtgaccgtgacctgcagaaacaaacagtggtccgagcctccaagatgtctggacccttgcgtggtgtccgaggaaaacatgaacaagaacaacatccagctgaagtggcggaacgacggcaagctgtatgccaagactggggacgccgtggaatttcagtgcaagttccctcacaaggccatgatcagcagccctccattcagagccatctgccaagagggcaagttcgagtaccccatctgcgag

SEQ ID NO: 135 - DNA, 1302 bp, Artificial Sequence
  Synthetic Construct
gaggattgcaatgagctgcctcctcggagaaacaccgagatcctgacaggctcttggagcgaccagacataccctgagggcacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcacactgaccggcggcaacgtgttcgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtacagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccccgccaccagaggcaataccgccaagtgtacaagcaccggctggatccctgctcctagatgcacactgaaacaggtgcagctggtggaatctggcggcggacttgtgaaacctggcggctctctgagactgtcttgtgccgccagcggcagaacctttagcagctacgccatgggatggttcagacaggcccctggcaaagagagagagttcgttagcgccatcagcggctctggcggcagcacatattacgccgatagcgtgaagggcagattcaccatcagccgggacaacgccaagaacagcctgtacctgcagatgaactccctgagagccgaggacaccgccgtgtactattgtgccgctgatctcggagatggcagctgggtcgactatgtgaatgccgagccttacgagtacgactactggggccagggcacactggtcaccgtgtcatct

SEQ ID NO: 136 - DNA, 1302 bp, Artificial Sequence
  Synthetic Construct
caggattgcaatgagctgcctcctcggagaaacaccgagatcctgacaggctcttggagcgaccagacataccctgagggcacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcacactgaccggcggcaacgtgttcgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtacagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccccgccaccagaggcaataccgccaagtgtacaagcaccggctggatccctgctcctagatgcacactgaaacaggtgcagctggtggaatctggcggcggacttgtgaaacctggcggctctctgagactgtcttgtgccgccagcggcagaacctttagcagctacgccatgggatggttcagacaggcccctggcaaagagagagagttcgttagcgccatcagcggctctggcggcagcacatattacgccgatagcgtgaagggcagattcaccatcagccgggacaacgccaagaacagcctgtacctgcagatgaactccctgagagccgaggacaccgccgtgtactattgtgccgctgatctcggagatggcagctgggtcgactatgtgaatgccgagccttacgagtacgactactggggccagggcacactggtcaccgtgtcatct

SEQ ID NO: 137 - DNA, 1359 bp, Artificial Sequence
  Synthetic Construct
gaggattgcaatgagctgcctcctcggagaaacaccgagatcctgacaggctcttggagcgaccagacataccctgagggcacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcacactgaccggcggcaacgtgttcgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtacagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccccgccaccagaggcaataccgccaagtgtacaagcaccggctggatccctgctcctagatgcacactgaaagaggtgcagctggttgagtctggcggcggacttgtgaaacctggcggaagcctgagactgtcttgtgccgccagcggcagaacctttagcagctacgccatgggatggttcagacaggcccctggcaaagagagagagttcgttagcgccatctctggcagcggcggcagcacatattacgccgattctgtgaagggcagattcaccatcagccgggacaacgccaagaacagcctgtacctgcagatgaactccctgagagccgaggacaccgccgtgtactattgtgccgctgatctcggagatggcagctgggtcgactatgtgaatgccgagccttacgagtacgactactggggccagggcacactggttacagttagttctggcggaggcggagcctgtaggggagacagaggcgatagaggcgacaggggagattgc

SEQ ID NO: 138 - DNA, 1548 bp, Artificial Sequence
  Synthetic Construct
gaggattgcaatgagctgcctcctcggagaaacaccgagatcctgacaggctcttggagcgaccagacataccctgagggcacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcacactgaccggcggcaacgtgttcgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtacagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccccgccaccagaggcaataccgccaagtgtacaagcaccggctggatccctgctcctagatgcaccctgaagccttgcgactaccccgatattaagcacggcggcctgtaccacgagaacatgagaaggccttacttccccgtggccgtgggcaagtactacagctactactgcgacgagcacttcgagacacctagcggcagctactgggatcacatccactgtacccaggatggctggtcccctgctgtgccttgtctggaagtgcagctggttgaatctggcggcggacttgtgaaacctggcggctctctgagactgtcttgtgccgccagcggcagaacctttagcagctacgccatgggatggttcagacaggcccctggcaaagagagagagttcgttagcgccatcagcggctctggcggcagcacatattacgccgatagcgtgaagggcagattcaccatcagccgggacaacgccaagaacagcctgtacctgcagatgaactccctgagagccgaggacaccgccgtgtactattgtgccgctgatctcggagatggcagctgggtcgactatgtgaatgccgagccttacgagtacgactactggggccagggcacactggttacagttagttctggcggaggcggagcctgtaggggagacagaggcgatagaggcgacaggggagattgc

SEQ ID NO: 139 - DNA, 1374 bp, Artificial Sequence
  Synthetic Construct
gaggattgcaatgagctgcctcctcggagaaacaccgagatcctgacaggctcttggagcgaccagacataccctgagggcacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcacactgaccggcggcaacgtgttcgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtacagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccccgccaccagaggcaataccgccaagtgtacaagcaccggctggatccctgctcctagatgtacacttaaaggcggaggcggagccgaggtgcaacttgttgaatctggcggcggactggttaagcctggcggatctctgagactgtcttgtgccgccagcggcagaacctttagcagctacgccatgggatggttcagacaggcccctggcaaagagagagagttcgttagcgccatctctggcagcggcggcagcacatattacgccgattctgtgaagggcagattcaccatcagccgggacaacgccaagaacagcctgtacctgcagatgaactccctgagagccgaggacaccgccgtgtactattgtgccgctgatctcggagatggcagctgggtcgactatgtgaatgccgagccttacgagtacgactactggggccagggcacactggttacagtttcttcaggtggcggaggggcctgcaggggagacagaggcgatagaggcgacaggggagattgc

(19) United States
(12) Patent Application Publication
(10) Pub. No.: US 2024/0262877 A1
(43) Pub. Date: Aug. 8, 2024

(54) KIDNEY ACTIVE FUSION PROTEINS AND METHODS OF TREATMENT USING THE SAME

(71) Applicant: Alexion Pharmaceuticals, Inc., Boston, MA (US)

(72) Inventors: Keith Bouchard, Unionville, CT (US); Jeffrey William Hunter, Wallingford, CT (US); Bruce A. Andrien, Jr., Guilford, CT (US); Sung-Kwon Kim, Cheshire, CT (US); Julian Chandler, Old Lyme, CT (US)

(73) Assignee: Alexion Pharmaceuticals, Inc., Boston, MA (US)

(21) Appl. No.: 18/561,469

(22) PCT Filed: May 24, 2022
(86) PCT No.: PCT/US2022/030658

§ 371 Date: Nov. 16, 2023

Related U.S. Application Data
(60) Provisional application No. 63/192,835, filed on May 25, 2021.

(51) Int. Cl. C07K 14/47; A61K 38/00; A61K 45/06; A61P 13/12; C07K 14/78

(52) U.S. Cl. CPC C07K 14/472; A61K 45/06; A61P 13/12; C07K 14/78; A61K 38/00; C07K 2317/22; C07K 2317/624; C07K 2319/30

(57) ABSTRACT

Described herein are fusion proteins which include factor H catalytic domains and may include VHH domains, factor H-related protein 5 domains, or integrin binding domains, and the use of such fusion proteins in methods of treatment of diseases mediated by complement alternative pathway activation or dysregulation, for example, kidney diseases.

### SEQUENCE LISTING

This application contains a Sequence Listing which has been submitted electronically in ASCII format and is hereby incorporated by reference in its entirety. Said ASCII copy, created on May 16, 2022, is named 50694-083WO2_Sequence_Listing_5_16_22_ST25 and is 140,215 bytes in size.

### BACKGROUND

The complement system plays a central role in the clearance of immune complexes and in immune responses to infectious agents, foreign antigens, virus-infected cells, and tumor cells. Complement activation occurs primarily by three pathways: the classical pathway, the lectin pathway, and the alternative pathway. The alternative pathway of complement is in a constant state of low-level activation. Uncontrolled activation or insufficient regulation of the complement alternative pathway (CAP) can lead to inflammation, cellular injury, and tissue damage. Local alternative pathway activation within the kidney is a contributor to renal pathology and loss of function. Thus, the complement alternative pathway has been implicated in the pathogenesis of a number of renal diseases. Inhibition or modulation of complement alternative pathway activity, in the absence of initiation of the lectin and classical pathway, has been recognized as a promising therapeutic strategy. For example, the alternative pathway plays a role in amplifying complement activation initiated from all three pathways. The number of treatment options available for diseases associated with the complement alternative pathway are limited. Thus, developing innovative strategies to treat diseases associated with complement alternative pathway activation or dysregulation, such as kidney diseases, which affect an estimated 37 million people in the U.S. alone, is a significant unmet need.

### SUMMARY OF THE DISCLOSURE

Described herein are fusion polypeptides that include a Factor H catalytic domain. The fusion proteins may be used to treat patients with diseases associated with complement alternative pathway activation or dysregulation, such as kidney diseases.

Provided herein is a fusion protein having the structure, from N-terminus to C-terminus of D1-L1-D2-L2-D3, wherein D1 includes a fragment of complement factor H (FH); L1 is absent, is a covalent bond, or is an amino acid sequence of at least one amino acid; D2 includes a VHH or is absent; L2 is absent, is a covalent bond, or is an amino acid sequence of at least one amino acid; and D3 is an integrin recognition domain. In some embodiments, D1 includes one or more (e.g., two, three, four, five or more) FH short consensus repeat (SCR) domains, optionally wherein the one or more SCR domains are selected from the group consisting of SCR 1, 2, 3, 4, 5, 6, 19, and 20. In some embodiments, the FH SCR domains are selected from the group consisting of SCR 1-4; 1-5; 1-6, 19, and 20; 1-5, 19, and 20; or 19 and 20.

In an embodiment, the VHH of D2 includes a single-domain antibody. In another embodiment, the VHH of D2 includes a camelid single domain antibody. In an embodiment, the integrin recognition domain of D3 includes an integrin recognition domain including an arginylglycylaspartic acid (RGD) peptide motif. In another embodiment, the integrin recognition domain of D3 includes a cyclo(RGD)4 peptide motif.

In an embodiment, L1 and L2 include the same amino acid sequence. In another embodiment, L1 and L2 include different amino acid sequences. In some embodiments, L1 and/or L2 are selected from the group consisting of: (G₄A)₂G₃AG₄S, G₄SDAA, (G₄A)₂G₄S, G₄AG₃AG₄S, GGGGAGGGGAGGGGS, GGGGSGGGGSGGGGS, G₄S, (G₄S)₂, (G₄S)₃, (G₄S)₄, (G₄S)₅, (G₄S)₆, (EAAAK)₃, PAPAP, G₄SPAPAP, PAPAPG₄S, GSTSGKSSEGKG, (GGGDS)₂, (GGGES)₂, GGGDSGGGGS, GGGASGGGGS, GGGESGGGGS, ASTKGP, ASTKGPSVFPLAP, G₃P, G₇P, PAPNLLGGP, G₆, G₁₂, APELPGGP, SEPQPQPG, (G₃S2)₃, GGGGGGGGGGGGS, GGGGSGGGGGGGGGS, (GGSSS)₃, (GS₄)₃, G₄A(G₄S)₂, G₄SG₄AG₄S, G₃AS(G₄S)₂, G₄SG₃ASG₄S, G₄SAG₃SG₄S, (G₄S)₂AG₃S, G₄SAG₃SAG₃S, G₄D(G₄S)₂, G₄SG₄DG₄S, (G₄D)₂G₄S, G₄E(G₄S)₂, G₄SG₄EG₄S, (G₄E)₂G₄S, G₄SDA, G₄A, and (G₄A)₃, such as G₄A. In some embodiments, L1 and/or L2 are selected from the group consisting of: (G₄A)₂G₃AG₄S, G₄SDAA, (G₄A)₂G₄S, G₄SDAA, (G₄S)₄, G₄AG₃AG₄S, G₄A, and (G₄A)₃.

In some embodiments, the fusion protein includes the FH SCR domains 1-5; L1 includes G₄A; D2 is absent; L2 is absent; and D3 includes cyclo(RGD)₄; D1 includes the FH SCR domains 1-5; L1 is absent; D2 includes the VHH; L2 includes G₄A; and D3 includes cyclo(RGD)₄; D1 includes the FH SCR domains 1-5; L1 includes G₄A; D2 is absent; L2 includes G₄A; and D3 includes cyclo(RGD)₄; D1 includes the FH SCR domains 1-5; L1 is absent; D2 includes a VHH; L2 includes G₄A; and D3 includes cyclo(RGD)₄; D1 includes the FH SCR domains 1-5; L1 is absent; D2 includes a VHH; L2 includes G₄A; and D3 includes cyclo(RGD)₄; D1 includes the FH SCR domains 1-6; L1 is absent; D2 includes a VHH; L2 includes G₄A; and D3 includes cyclo(RGD)₄; or D1 includes the FH SCR domains 1-5; L1 includes G₄A; D2 includes a VHH; L2 includes G₄A; and D3 includes cyclo(RGD)₄.

In some embodiments, the fusion protein has an amino acid sequence of SEQ ID NO: 4, or a variant thereof having up to 10 amino acid (e.g., 1, 2, 3, 4, 5, 6, 7, 8, and 9 amino acid) substitutions, additions, or deletions; has an amino acid sequence of SEQ ID NO: 5, or a variant thereof having up to 10 amino acid (e.g., 1, 2, 3, 4, 5, 6, 7, 8, and 9 amino acid) substitutions, additions, or deletions; has an amino acid sequence of SEQ ID NO: 8, or a variant thereof having up to 10 amino acid (e.g., 1, 2, 3, 4, 5, 6, 7, 8, and 9 amino acid) substitutions, additions, or deletions; has an amino acid sequence of SEQ ID NO: 9, or a variant thereof having up to 10 amino acid (e.g., 1, 2, 3, 4, 5, 6, 7, 8, and 9 amino acid) substitutions, additions, or deletions; has an amino acid sequence of SEQ ID NO: 13, or a variant thereof having up to 10 amino acid (e.g., 1, 2, 3, 4, 5, 6, 7, 8, and 9 amino acid) substitutions, additions, or deletions; or has an amino acid sequence of SEQ ID NO: 14, or a variant thereof having up to 10 amino acid (e.g., 1, 2, 3, 4, 5, 6, 7, 8, and 9 amino acid) substitutions, additions, or deletions; has an amino acid sequence of SEQ ID NO: 15, or a variant thereof having up to 10 amino acid (e.g., 1, 2, 3, 4, 5, 6, 7, 8, and 9 amino acid) substitutions, additions, or deletions.

In some embodiments, the fusion protein has an amino acid sequence with at least 85% (e.g., at least 90%, 95%, and 99%) sequence identity to SEQ ID NO: 4; has an amino acid sequence with at least 85% (e.g., at least 90%, 95%, and 99%) sequence identity to SEQ ID NO: 5; has an amino acid sequence with at least 85% (e.g., at least 90%, 95%, and 99%) sequence identity to SEQ ID NO: 8; has an amino acid sequence with at least 85% (e.g., at least 90%, 95%, and 99%) sequence identity to SEQ ID NO: 9; has an amino acid sequence with at least 85% (e.g., at least 90%, 95%, and 99%) sequence identity to SEQ ID NO: 13; has an amino acid sequence with at least 85% (e.g., at least 90%, 95%, and 99%) sequence identity to SEQ ID NO: 14; has an amino acid sequence with at least 85% (e.g., at least 90%, 95%, and 99%) sequence identity to SEQ ID NO: 15.

In another aspect, the disclosure provides a fusion protein including the structure, from N-terminus to C-terminus of D1-L1-D2, wherein D1 includes a FH fragment, such as FH1-5; L1 includes a linker or is absent; and D2 includes a factor H-related protein 5 (FHRP5) domain, such as FHRP domains 7 and 8. In an embodiment, L1 is selected from the group consisting of: G₄A, (G₄A)₃, (G₄A)₂G₃AG₄S, G₄SDAA, (G₄A)₂G₄S, G₄AG₃AG₄S, GGGGAGGGGAGGGGS, GGGGSGGGGSGGGGS, G₄S, (G₄S)₂, (G₄S)₃, (G₄S)₄, (G₄S)₅, (G₄S)₆, (EAAAK)₃, PAPAP, G₄SPAPAP, PAPAPG₄S, GSTSGKSSEGKG, (GGGDS)₂, (GGGES)₂, GGGDSGGGGS, GGGASGGGGS, GGGESGGGGS, ASTKGP, ASTKGPSVFPLAP, G₃P, G₇P, PAPNLLGGP, G₆, G₁₂, APELPGGP, SEPQPQPG, (G₃S2)₃, GGGGGGGGGGGGS, GGGGSGGGGGGGGGS, (GGSSS)₃, (GS₄)₃, G₄A(G₄S)₂, G₄SG₄AG₄S, G₃AS(G₄S)₂, G₄SG₃ASG₄S, G₄SAG₃SG₄S, (G₄S)₂AG₃S, G₄SAG₃SAG₃S, G₄D(G₄S)₂, G₄SG₄DG₄S, (G₄D)₂G₄S, G₄E(G₄S)₂, G₄SG₄EG₄S, (G₄E)₂G₄S, and G₄SDA, such as G₄A. In some embodiments, L1 is selected from the group consisting of: G₄A, and (G₄A)₃, (G₄A)₂G₃AG₄S, G₄SDAA, (G₄A)₂G₄S, G₄SDAA, (G₄S)₄, and G₄AG₃AG₄S.

In an embodiment, the fusion protein has an amino acid sequence of SEQ ID NO: 6, or a variant having up to 10 amino acid (e.g., 1, 2, 3, 4, 5, 6, 7, 8, 9, and 10 amino acids) substitutions, additions, or deletions; or has an amino acid sequence of SEQ ID NO: 10, or a variant having up to 10 amino acid (e.g., 1, 2, 3, 4, 5, 6, 7, 8, 9, and 10 amino acid) substitutions, additions, or deletions. In some embodiments, the fusion protein has an amino acid sequence with at least 85% (e.g., at least 90%, 95, and 99%) sequence identity to SEQ ID NO: 6; or has an amino acid sequence with at least 85% (e.g., at least 90%, 95, and 99%) sequence identity to SEQ ID NO: 10.

In another aspect, the disclosure provides a fusion protein including the structure, from N-terminus to C-terminus: of D1-L1-D2-L2-D3, wherein D1 includes an integrin recognition domain, such as cyclo(RGD)₄; L1 includes a linker or is absent; D2 is a VHH, such as a single domain antibody, L2 is a linker or is absent; and D3 is a FH fragment, such FH1-5. In some embodiments, the fusion protein has a C-terminal His tag. In an embodiment, L1 and L2 include the same amino acid sequence. In another embodiment, L1 and L2 include different amino acid sequences. In some embodiments, L1 and/or L2 are selected from the group consisting of: G₄A, (G₄A)₃, (G₄A)₂G₃AG₄S, G₄SDAA, (G₄A)₂G₄S, G₄AG₃AG₄S, GGGGAGGGGAGGGGS, GGGGSGGGGSGGGGS, G₄S, (G₄S)₂, (G₄S)₃, (G₄S)₄, (G₄S)₅, (G₄S)₆, (EAAAK)₃, PAPAP, G₄SPAPAP, PAPAPG₄S, GSTSGKSSEGKG, (GGGDS)₂, (GGGES)₂, GGGDSGGGGS, GGGASGGGGS, GGGESGGGGS, ASTKGP, ASTKGPSVFPLAP, G₃P, G₇P, PAPNLLGGP, G₆, G₁₂, APELPGGP, SEPQPQPG, (G₃S2)₃, GGGGGGGGGSGGGS, GGGGSGGGGGGGGGS, (GGSSS)₃, (GS₄)₃, G₄A(G₄S)₂, G₄SG₄AG₄S, G₃AS(G₄S)₂, G₄SG₃ASG₄S, G₄SAG₃SG₄S, (G₄S)₂AG₃S, G₄SAG₃SAG₃S, G₄D(G₄S)₂, G₄SG₄DG₄S, (G₄D)₂G₄S, G₄E(G₄S)₂, G₄SG₄EG₄S, (G₄E)₂G₄S, and G₄SDA, such as G₄A. In some embodiments, L1 and/or L2 are selected from the group consisting of: G₄A, (G₄A)₃, (G₄A)₂G₃AG₄S, G₄SDAA, (G₄A)₂G₄S, G₄SDAA, (G₄S)₄, and G₄AG₃AG₄S.

In some embodiments, the fusion protein has an amino acid sequence of SEQ ID NO: 2, or a variant having up to 10 amino acid (e.g., 1, 2, 3, 4, 5, 6, 7, 8, and 9 amino acids) substitutions, additions, or deletions; or has an amino acid sequence of SEQ ID NO: 3, or a variant having up to 10 amino acid (e.g., 1, 2, 3, 4, 5, 6, 7, 8, and 9 amino acids) substitutions, additions, or deletions. In some embodiments, the fusion protein has an amino acid sequence with at least 85% (e.g., at least 90%, 95% or 99%) sequence identity to SEQ ID NO: 2; or has an amino acid sequence with at least 85% (e.g., at least 90%, 95% or 99%) sequence identity to SEQ ID NO: 3.

In another aspect, the disclosure provides a fusion protein including the structure, from N-terminus to C-terminus, of D1-D2 or D2-D1, wherein D1 is a VHH, such as a single domain antibody, and D2 is a FH fragment, such as FH1-5. In some embodiments, the fusion protein has a C-terminal His tag. In some embodiments, the fusion protein has an amino acid sequence of SEQ ID NO: 1, or a variant having up to 10 amino acid (e.g., 1, 2, 3, 4, 5, 6, 7, 8, and 9 amino acids) substitutions, additions, or deletions; or has an amino acid sequence of SEQ ID NO: 7, or a variant having up to 10 amino acid (e.g., 1, 2, 3, 4, 5, 6, 7, 8, and 9 amino acids) substitutions, additions, or deletions. In some embodiments, the fusion protein has an amino acid sequence with at least 85% (e.g., at least 90%, 95% or 99%) sequence identity to SEQ ID NO: 1; or has an amino acid sequence with at least 85% (e.g., at least 90%, 95% or 99%) sequence identity to SEQ ID NO: 7. In some embodiments, the fusion protein has an amino acid sequence of SEQ ID NO: 11, or a variant having up to 10 amino acid (e.g., 1, 2, 3, 4, 5, 6, 7, 8, and 9 amino acids) substitutions, additions, or deletions; or has an amino acid sequence of SEQ ID NO: 12, or a variant having up to 10 amino acid (e.g., 1, 2, 3, 4, 5, 6, 7, 8, and 9 amino acids) substitutions, additions, or deletions. In some embodiments, the fusion protein has an amino acid sequence with at least 85% (e.g., at least 90%, 95% or 99%) sequence identity to SEQ ID NO: 11; or has an amino acid sequence with at least 85% (e.g., at least 90%, 95% or 99%) sequence identity to SEQ ID NO: 12.

In an embodiment, the fusion protein has an increased intrarenal residence time relative to the fusion protein lacking the VHH domain.

In another aspect, the disclosure provides a pharmaceutical composition including any one of the fusion proteins described herein and a pharmaceutically acceptable carrier.

In another aspect the disclosure provides a polynucleotide encoding any one of the fusion proteins described herein.

In another aspect, the disclosure provides a host cell including a vector including the polynucleotide described herein.

In another aspect, the disclosure provides a host cell including the polynucleotide described herein or the vector described herein.

In another aspect, the disclosure provides a method of producing any one of the fusion proteins described herein including the steps of culturing one or more host cells including one or more nucleic acid molecules capable of expressing the fusion protein under conditions suitable for expression of the fusion protein. In some embodiments, the method further includes the step of obtaining the fusion protein from the cell culture or culture medium.

In another aspect, the disclosure provides a method of treating a disease mediated by complement alternative pathway activation or dysregulation including administering an effective amount of a composition including any one of the fusion proteins described herein, the pharmaceutical composition described herein, the polynucleotide described herein, the vector described herein, or the host cell described herein to a subject in need thereof. In some embodiments, the fusion protein is formulated as a pharmaceutical composition with at least one (e.g., at least one, two, five, or ten) pharmaceutically acceptable carrier. In an embodiment, the composition is lyophilized. In another embodiment, the composition is rehydrated prior to administration. In another embodiment, the at least one (e.g., at least one, two, five, or ten) pharmaceutically acceptable carrier is saline. In some embodiments, the composition is formulated for daily, weekly, or monthly administration.

In some embodiments, the composition is formulated for intravenous, subcutaneous, intramuscular, oral, nasal, sublingual, intrathecal, and intradermal administration. In some embodiments, the composition is formulated for administration at a dosage of between about 0.1 mg/kg to about 150 mg/kg (e.g., about 0.5-150 mg/kg, 1-150 mg/kg, 10⁻¹⁵⁰ mg/kg, 25-150 mg/kg, 50-150 mg/kg, 100-150 mg/kg, 125-150 mg/kg, 0.1-125 mg/kg, 0.1-100 mg/kg, 0.1-50 mg/kg, 0.1-25 mg/kg, 0.1-10 mg/kg, 0.1-5 mg/kg, and 0.1-1 mg/kg). In some embodiments, the composition is formulated for administration in combination with an additional therapeutic agent.

In some embodiments, the disease mediated by complement alternative pathway activation or dysregulation is kidney disorders, focal segmental glomerulosclerosis (FSGS), IgA nephropathy, minimal change disease (MCD), diabetic nephropathy, Alport syndrome, lupus nephritis, membranous nephropathy, acute kidney injury, Goodpasture syndrome, nephrotic syndrome, chronic proteinuria, chronic kidney disease, C3 glomerulopathy (C3G), dense deposit disease, membranoproliferative glomerulonephritis, glomerulonephritis, polycystic kidney disease, hypertensive nephropathy, nephrosclerosis, atypical hemolytic uremic syndrome (aHUS), ischemia reperfusion injury, or rejection of a transplanted organ, such as a kidney. In some embodiments, the subject is a mammal. In some embodiments, the mammal is a human.

In another aspect, the disclosure provides a kit including a composition selected from any one of the fusion proteins described herein, the pharmaceutical composition described herein, the polynucleotide described herein, the vector described herein, or the host cell described herein. In some embodiments, the kit further includes instructions for administering an effective amount of the composition to a subject in need thereof.

### BRIEF DESCRIPTION OF THE DRAWINGS

FIGS. 1A-1B are schematic diagrams illustrating complement factor H (FH) fusion proteins of Formulas I and III, which include an integrin recognition domain (FIG. 1A), and Factor H fusion proteins of Formula II (FIG. 1B), which include a fragment of FHRP5.

FIG. 2A is a graph showing assay results for comparative inhibition of CAP-mediated hemolysis by Compound A and factor H SCRs 1-5.

FIG. 2B is a graph showing assay results for comparative inhibition of CAP-mediated hemolysis by Compounds D, H, E, and I.

FIG. 2C is a graph showing assay results for comparative inhibition of CAP-mediated hemolysis by Compounds E, I, and reference protein 6, which is an anti-HSA factor H-VHH fusion protein used as a positive control.

FIG. 2D is a graph showing assay results for comparative inhibition of CAP-mediated hemolysis by Compounds E, M, N, and O.

FIG. 3 is a set of whole body and kidney in vivo images showing of wild-type mice after treatment with Compound B. The images were produced using a LI-COR Odyssey microscope.

FIG. 4 is a graph showing serum levels in ng/mL for the indicated fusion proteins at 1 hour and 24 hours after administration to wild-type mice.

FIG. 5 is a graph showing proteinuria levels in an adriamycin nephropathy model of FSGS, in wild-type Balb/c mice, following administration of fusion proteins with an intravenous dose performed on day 0 and subcutaneous dosing on days 7, 9, 11, and 13. The data are shown as means + or − of the standard error of the mean (n=4-9). Statistically significant differences compared to vehicle are denoted by *p<0.05 and ***p<0.001, and statistically significant differences compared to vehicle and adriamycin are denoted by †p<0.5.

FIG. 6 is a graph showing albuminuria levels an adriamycin nephropathy model of FSGS, in wild-type Balb/c mice, following administration of fusion proteins with an intravenous dose performed on day 0 and subcutaneous dosing on days 7, 9, 11, and 13. The data are shown as means + or − of the standard error of the mean (n=4-9). Statistically significant differences compared to vehicle are denoted by *p<0.05 and ***p<0.001, and statistically significant differences compared to vehicle and adriamycin are denoted by †p<0.5.

FIG. 7 is a graph showing blinded tubular protein scoring of Masson's trichrome-stained adriamycin nephropathy mouse kidneys, scored 1-5 according to accepted methods. N=8-10. Statistically significant differences compared to vehicle using multiple t-tests are denoted by **p<0.01, and using ANOVA with multiple comparisons denoted by #p<0.5.

FIG. 8A is a set of images showing exemplary immunofluorescent evaluation of kidney sections for C3 deposition, in an adriamycin nephropathy model, in wild-type Balb/c mice, 7 days following administration of Compound E. Other molecules gave similar results, or were closer to vehicle negative control.

FIG. 8B is a graph showing pixel mean intensity for FIG. 9 results; C3 pixel mean intensity values represent average signal intensity within select region of interest/renal medulla at day 14 (day 7 post treatment).

FIG. 9A is a graph showing urine protein/creatinine ratios in male Balb/c mice with adriamycin-induced kidney disease following treatment with fusion proteins, with an intravenous dose performed on day 0 and subcutaneous dosing on days 7, 9, 11, and 13. The data are shown as means + or − of the standard error of the mean (n=4-9). Statistically significant differences compared to vehicle are denoted by ***p<0.001. There were no statistically significant differences compared to vehicle and adriamycin. Positive trends were observed.

FIG. 9B is a graph showing urine albumin/creatinine ratios following treatment with fusion proteins, with an intravenous dose performed on day 0 and subcutaneous dosing on days 7, 9, 11, and 13. The data are shown as means + or − of the standard error of the mean (n=4-9). Statistically significant differences compared to vehicle are denoted by ***p<0.001. There were no statistically significant differences compared to vehicle and adriamycin.

FIG. 10A is a Western blot showing an SDS-PAGE gel of purified Compounds D and E.

FIG. 10B is a graph showing a hydrophobic interaction chromatogram of Compound E.

FIG. 11 is a graph showing the mass spectrometry of Compound E, which shows the molecular weight of Compound E to be about 50 kDa. A minor peak of +162 Da was observed and is likely due to glycation.

FIG. 12 is a graph showing a melting curve for Compound E using Dynamic Light Scattering.

FIG. 13A is a graph showing the retention time of Compound E at 0 days at 37° C. using size exclusion chromatography to measure the relative stability of the compound.

FIG. 13B is a graph showing the retention time of Compound E after 14 days at 37° C. using size exclusion chromatography to measure the relative stability of the compound.

FIG. 14A is a graph showing the retention time of Compound E after 0 days at 37° C. using hydrophobic interaction chromatography to measure the relative stability of the compound.

FIG. 14B is a graph showing the retention time of Compound E after 14 days at 37° C. using hydrophobic interaction chromatography to measure the relative stability of the compound.

FIG. 15A is a graph showing the aligned times of non-reduced Compound E after 0 days and 14 days at 37° C. using capillary electrophoresis-SDS chromatography to measure the relative stability of the compound.

FIG. 15B is a graph showing the aligned times of reduced Compound E after 0 days and 14 days at 37° C. using capillary electrophoresis-SDS chromatography to measure the relative stability of the compound.

FIG. 16 is a graph showing a chromatogram signature of Compound E obtained using iso-electric capillary electrophoresis (ICE).

FIG. 17 is a graph showing the mass spectra of Compound E measured after 0 days, 3 days, 7 days, and 14 days at 37° C. to characterize the stability of the compound at room temperature.

FIG. 18A is a graph showing a binding curve of Compound E and Compound K to C3b as compared to factor H (fH) over 0 to 2500 seconds.

FIG. 18B is a graph showing a 40-second, blown-up view of the binding curve of FIG. 18A, wherein the t=0 seconds timepoint on FIG. 18B corresponds to the t=720 seconds timepoint on FIG. 18A.

FIG. 19 is a graph showing assay results for comparative inhibition of fluid-phase CAP activation by Compound E in the Complement system Alternative Pathway WIESLAB® across two lots of normal human serum (NHS).

FIG. 20 is a graph showing composite single-dose serum pharmacokinetics (PK) data of Compound E following subcutaneous (SC) administration to wild-type male C57BI/6 mice across two separate studies.

FIG. 21A is a graph showing serum pharmacokinetics of Compound E following intravenous (IV) or SC administration to female cynomolgus monkeys. Included within each graph are comparative PK profiles across a range of dose levels following both the initial dose given on study day 0 and a fourth dose administered on study day 12.

FIG. 21B includes the data from FIG. 21A re-plotted to compare equivalent dose levels given by IV or SC route of administration.

### DEFINITIONS

As used herein, the term “about” refers to a value that is within 10% above or below the value being described.

As used herein, “administering” and “administration” refer to any method of providing a pharmaceutical preparation to a subject. Fusion proteins may be administered by any method known to those skilled in the art. Suitable methods for administering the fusion protein may be, for example, orally, by injection (e.g., intravenously, intraperitoneally, intramuscularly, intravitreally, and subcutaneously), drop infusion preparations, inhalation, intranasally, and the like. In some embodiments, administration is via intravenous and/or subcutaneous infusions. Fusion proteins prepared, as described herein, may be administered in various forms depending on the disorder to be treated and the age, condition, and body weight of the subject, as is known in the art. A preparation can be administered prophylactically; that is, administered to decrease the likelihood of developing a disease or condition.

As used herein, the terms “binding affinity,” “specifically binds,” “affinity” refer to the strength of the total noncovalent interactions between a single binding site of a molecule and its binding partner. Unless otherwise indicated, as used herein, “binding affinity” refers to intrinsic binding affinity, which reflects a specific interaction between members of a binding pair. The affinity of a molecule X for its partner Y can generally be represented by the dissociation constant (Kd). Affinity can be measured by standard methods known in the art, including those described herein. A low-affinity complex contains a molecule that generally tends to dissociate readily from its binding partner, whereas a high-affinity complex contains a molecule that generally tends to remain bound to its binding partner for a longer duration. “Specifically binds” refers to molecules and binding partner pairs that have a Kd of at least 1×10⁻⁶ M or lower (e.g., in the range of 1×10⁻⁶ M to 1×10⁻¹² M, such as 1×10⁻⁷ M, 1×10⁻⁸ M, 1×10⁻⁹ M, 1×10⁻¹⁰ M, 1×10⁻¹¹ M, and 1×10⁻¹² M).

As used herein, the term “antibody” refers to an immunoglobulin molecule that specifically or substantially specifically binds to, or is immunologically reactive with, a particular antigen. The antibody can be, for example, a natural or artificial mono- or polyvalent antibody including, but not limited to, a polyclonal, monoclonal, multi-specific, human, humanized, or chimeric antibody. An antibody may be a genetically engineered or otherwise modified form of an antibody, including but not limited to, heteroconjugate antibodies (e.g., bi-, tri-, and tetra-specific antibodies, diabodies, triabodies, and tetrabodies), and antigen binding fragments of antibodies, including, for example, single domain, VHH, Fab′, F(ab′)₂, Fab, Fv, rIgG and scFv fragments.

As used herein, the term “complement alternative pathway” refers to one of three pathways of complement activation (the others being the classical pathway and the lectin pathway).

As used herein, the term “complement alternative pathway activation or dysregulation” refers to any aberration in the ability of the complement alternative pathway to provide host defense against pathogens and clear immune complexes and damaged cells for immunoregulation. Complement alternative pathway activation or dysregulation can occur in the fluid phase and at the cell surface. Complement alternative pathway activation or dysregulation can lead to excessive complement activation or insufficient regulation, both causing tissue injury.

As used herein, the term “disease” refers to an interruption, cessation, or disorder of body functions, systems, or organs. Disease(s) or disorders of interest include those that would benefit from treatment with a fusion protein or by a method described herein. Non-limiting examples of diseases or disorders to be treated herein are diseases or disorders mediated by complement alternative pathway activation or dysregulation include, but are not limited to, kidney disorders, focal segmental glomerulosclerosis (FSGS), IgA nephropathy, minimal change disease (MCD), diabetic nephropathy, Alport syndrome, lupus nephritis, membranous nephropathy, acute kidney injury, Goodpasture syndrome, nephrotic syndrome, chronic proteinuria, chronic kidney disease, C3 glomerulopathy (C3G), dense deposit disease, glomerulonephritis, membranoproliferative glomerulonephritis, polycystic kidney disease, hypertensive nephropathy, nephrosclerosis, atypical hemolytic uremic syndrome (aHUS), ischemia reperfusion injury, or rejection of a transplanted organ, such as a kidney. In some embodiments, the disease is FSGS.

As used herein, “Factor H” refers to a protein component of the complement alternative pathway encoded by the complement factor H gene (“FH;” NM000186; GeneID:3075; UniProt ID P08603; Ripoche, J. et al., Biochem. J., 249:593-602, 1988) (SEQ ID NO: 123). Factor H is translated as a 1,213 amino acid precursor polypeptide that is processed by removal of an 18 amino acid signal peptide, resulting in the mature factor H protein (amino acids 19-1231). Factor H consists of 20 short complement regulator (SCR) domains. Amino acids 1-18 comprise the signal peptide, residues 21-80 comprise SCR1 (SEQ ID NO: 24, residues 85-141 comprise SCR 2 (SEQ ID NO: 25), residues 146-205 comprise SCR3 (SEQ ID NO: 26), residues 201-262 comprise SCR 4 (SEQ ID NO: 27), residues 267-320 comprise SCR 5 (SEQ ID NO: 28), residues 326-384 comprise SCR 6 (SEQ ID NO: 29). Factor H regulates complement activation on self-cells by possessing both cofactor activity for the factor I-mediated C3b cleavage, and decay accelerating activity against the alternative pathway C3 convertase, C3bBb.

Cleavage of C3 results initially in the generation and deposition of C3b on the activating cell surface. The C3b fragment is involved in the generation of enzymatic complexes that amplify the complement cascade. On a cell surface, C3b is rapidly converted to inactive iC3b, for example when deposited on a host surface containing regulators of complement activation (i.e., most host tissue). Even in the absence of membrane-bound complement regulators, substantial levels of iC3b are formed because of the action of serum factor H and serum factor I. iC3b is subsequently digested to the membrane-bound fragments C3dg and then C3d by factor I and other proteases and cofactors, but this process is relatively slow.

As used herein, the term “Factor H-Related Protein 5” or “FHRP5” refers to a protein component of the complement alternative pathway encoded by the complement factor H-related protein 5 gene (“CFHR5;” NM_030787.3; Gene ID: 81494; UniProt ID: Q9BXR6) (SEQ ID NO: 124). FHRP5 has nine SCRs. The first two SCRs have heparin binding properties, a region within SCRs 5-7 have heparin binding and C reactive protein binding properties, and the two C-terminal SCRs are similar to a complement component 3 b (C3b) binding domain. FHRP5 co-localizes with C3, binds C3b in a dose-dependent manner, and is recruited to tissues damaged by C-reactive protein.

As used herein, the term “fragment” refers to less than 100% of the amino acid sequence of a full-length reference protein (e.g., 99%, 90%, 80%, 70%, 60%, 50%, 40%, 30%, 20%, 10%, of the full-length sequence etc.), but including, e.g., 5, 10, 20, 25, 30, 35, 40, 45, 50, 100, 150, 200, 250, 300, 350, or more amino acids. A fragment can be of sufficient length such that a desirable function of the full-length protein is maintained. For example, the regulation of the complement alternative pathway in the fluid phase by fragments of, for example, factor H, is maintained. Such fragments are “biologically active fragments.”

As used herein, a “functional fragment” or a “biologically active fragment” refers to a fragment, or portion, of a protein having some or all of the activities of the full-length protein. For example, a functional or biologically active fragment of factor H, refers to any fragment of a factor H protein having some or all of the activities of factor H, e.g., complement alternative pathway regulatory activity of the full-length factor H protein. Examples include, but are not limited to, factor H fragments, joined from N-terminus to C terminus, containing the following SCRs: [1-4], [1-5], [1-6], [1-7], [1-20], [19-20], [1-4 and 19-20], and [1-5] and [19-20]. A functional fragment” or a “biologically active fragment” of FHRP5 protein is one having some or all of the activities of FHRP5, e.g., complement alternative pathway regulatory activity of the full-length FHRP5 protein. Examples include, but are not limited to, FHRP5 fragments, from N-terminus to C-terminus, containing the following SCRs: [7-8]. As used herein, the term “fused” or “joined” refers to the combination or attachment of two or more elements, components, or protein domains, e.g., polypeptides, by means including chemical conjugation, recombinant means, and chemical bonds, e.g., disulfide bonds and amide bonds. For example, two single polypeptides can be joined to form one contiguous protein structure through recombinant expression, chemical conjugation, a chemical bond, a peptide linker, or any other means of covalent linkage.

As used herein, the term “fusion protein” refers to a composite polypeptide made up of two (or more) distinct, heterologous polypeptides. The heterologous polypeptides can either be full-length proteins or fragments of full-length proteins. Fusion proteins herein can be prepared by either synthetic or recombinant techniques known in the art.

As used herein, the term “host cell” refers to any kind of cellular system that can be engineered to generate the fusion proteins described herein. Non-limiting examples of host cells include Expi CHO-S, Expi 293 F, HEK, HEK 293, HT-1080, CHO, Pichia pastoris, Saccharomyces cerevisiae, and transformable insect cells such as High Five, Sf9, and Sf21 cells.

As used herein, the term “integrin recognition motif” refers to a polypeptide oligomer of repeating arginylglycylaspartic acid moieties, e.g., (RGD)₁₋₈, such as (RGD)₁₋₄ (SEQ ID NO: 21). In some embodiments, the arginylglycylaspartic acid moieties may be cyclized.

As used herein, the term “intrarenal residence time” refers to a time period during which a compound, such as Compounds A-O described herein, is present in extravascular compartments, for example along the kidney epithelium or within Bowman's capsule within the kidney. The intrarenal residence time may be measured using longitudinal in vivo imaging. For example, in animal studies, the IVIS Spectrum Imaging System (PerkinElmer Inc., Waltham, MA) can be used for image acquisition. Fluorescent imaging analysis can be performed using Living Image 4.5.1 software (PerkinElmer Inc., Waltham, MA), with automatic 2D epi-illumination exposure settings, field of view (FOV) C, F/Stop 2, medium binning and 800 nm emission/750 nm excitation filters, with subjects receiving, for example, 1 mg/kg of AlexaFluor 750-labeled test article via intravenous injection. In clinical settings, for example, longitudinal in vivo imaging may be accomplished using radiolabeled test article and PET or SPECT imaging.

As used herein, the terms “linker,” “L1,” and “L2” refer to a linkage between two elements, e.g., polypeptides or protein domains. A linker can be a covalent bond. A linker can also be a molecule of any length that can be used to couple, for example, a factor H fragment and/or a VHH and/or an integrin recognition motif. A linker also refers to a moiety (e.g., a polyethylene glycol (PEG) polymer) or an amino acid sequence (e.g., a 1-200 amino acid, 1-150 amino acid, 1-100, a 5-50 amino acid, or a 1-10 amino acid sequence, such as amino acids with smaller side chains and/or flexible amino acid sequences) occurring between two polypeptides or polypeptide domains to provide space and/or flexibility between the two polypeptides or polypeptide domains. An amino acid linker may be part of the primary sequence of a polypeptide (e.g., joined to the linked polypeptides or polypeptide domains via the polypeptide backbone). Non-limiting examples include (G₄A)₂G₄S, G₄A, (G₄A)₃, and (G₄A)₂G₃AG₄S (SEQ ID NOS: 32, 80, 81, and 30).

As used herein, the term “patient in need thereof” or “subject in need thereof,” refers to a subject in need of treatment, e.g., based on the presence of a disease or disorder (e.g., one or more symptoms of the disease or disorder). A subject can be identified as having a need for treatment of a disease or disorder (e.g., kidney disorders, FSGS, IgA nephropathy, MCD, diabetic nephropathy, Alport syndrome, lupus nephritis, membranous nephropathy, acute kidney injury, Goodpasture syndrome, nephrotic syndrome, chronic proteinuria, chronic kidney disease, C3G, dense deposit disease, glomerulonephritis, membranoproliferative glomerulonephritis, polycystic kidney disease, hypertensive nephropathy, nephrosclerosis, aHUS, ischemia reperfusion injury, or rejection of a transplanted organ, such as a kidney) prior to administration of a treatment. In some embodiments, the disease is FSGS, and the need for treatment is based upon an earlier diagnosis by a person of skill in the art (e.g., a physician). For example, a patient is a mammal, such as a human.

The terms “peptide”, “polypeptide” and “protein” are used interchangeably herein to refer to polymers of amino acids of any length. The terms also encompass an amino acid polymer that has been modified, for example, by disulfide bond formation, glycosylation, acetylation, phosphorylation, lipidation, or conjugation with a labeling component, among others.

“Percent (%) sequence identity,” with respect to a reference polynucleotide or polypeptide sequence, is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software, such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

By “pharmaceutical composition” is meant any composition that contains a therapeutically or biologically active agent (e.g., fusion protein) that is suitable for administration to a subject. Any of these formulations can be prepared by well-known and accepted methods in the art. See, for example, Remington: The Science and Practice of Pharmacy (21st ed.), ed. A. R. Gennaro, Lippincott Williams & Wilkins, 2005, and Encyclopedia of Pharmaceutical Technology, ed. J. Swarbrick, Informa Healthcare, 2006, each of which is hereby incorporated by reference.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response, and other problem complications commensurate with a reasonable benefit/risk ratio.

The terms “polynucleotide” and “nucleic acid” are used interchangeably to refer to a polymeric form of nucleotides of any length, including deoxyribonucleotides, ribonucleotides, or analogs thereof. A polynucleotide may include modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the disclosure described herein that is a double-stranded polynucleotide encompasses both the double-stranded form and each of the two complementary single-stranded forms known or predicted to make up the double-stranded form.

As used herein, the terms “short complement regulator”, or “SCR”, also known as “short consensus repeat”, “sushi domains,” or “complement control protein” or “CCP,” describe domains found in all regulators of complement activation (RCA) gene clusters that contribute to their ability to regulate complement activation in the blood or on the cell surface to which they specifically bind. SCRs typically are composed of about 60 amino acids, with four cysteine residues disulfide bonded in a 1-3, 2-4 arrangement and a hydrophobic core built around an almost invariant tryptophan residue. SCRs are found in proteins including, but not limited to, factor H and FHRP5.

As used herein, the terms “single domain antibody” and “VHH” define molecules formed by a single immunoglobulin domain. Single domain antibodies include antibodies whose complementary determining regions (“CDRs”) are part of a single domain polypeptide. Single domain antibodies often include an antibody or antigen binding fragment thereof that specifically binds a single antigen (e.g., the VHH antibody binds an antigen with a K_{D} of 1×10⁻⁶ M or lower, e.g., a K_{D} in the range of 1×10⁻⁶ M to 1×10⁻¹² M, such as a K_{D} of 1×10⁻⁷ M, 1×10⁻⁸ M, 1×10⁻⁹ M, 1×10⁻¹⁰ M, 1×10⁻¹¹ M, and 1×10⁻¹² M). Generally, the antigen binding site of an immunoglobulin single variable domain is formed by no more than three CDRs. The single variable domain may, for example, include a light chain variable domain sequence (a VL sequence) or a suitable fragment thereof; or a heavy chain variable domain sequence (e.g., a VH sequence or VHH sequence), or a suitable fragment thereof. Such antibodies can be derived, for example, from antibodies raised in Camelidae species, for example, in a camel, dromedary, llama, alpaca, or guanaco. Additional antibodies include, for example, immunoglobulin new antigen receptor (IgNAR) of cartilaginous fishes (e.g., sharks, e.g., nurse sharks). Other species besides Camelidae and cartilaginous fishes may produce antibodies whose CDRs are part of a single polypeptide. Antibodies can be prepared by either synthetic or recombinant techniques known in the art.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

By “therapeutically effective amount” is meant the amount of a composition administered to improve, inhibit, or ameliorate a condition of a subject, or a symptom of a disorder or disease in a clinically relevant manner. Any improvement in the subject is considered sufficient to achieve treatment. In some embodiments, an amount sufficient to treat is an amount that reduces, inhibits, or prevents the occurrence or one or more symptoms of the disease or disorder (e.g., a disease or disorder mediated by complement alternative pathway activation or dysregulation) or is an amount that reduces the severity of, or the length of time during which a subject suffers from one or more symptoms of the disease or disorder, for example, any disease or disorder mediated by CAP activation or dysregulation, (e.g., by at least about 10%, about 20%, or about 30%, such as by at least about 50%, about 60%, or about 70%, and for example by at least about 80%, about 90%, about 95%, about 99%, or more, relative to a control subject that is not treated with a composition described herein). An effective amount of the pharmaceutical composition used to practice the methods described herein (e.g., the treatment of kidney diseases) may vary depending upon the manner of administration and the age, body weight, and general health of the subject being treated. A physician or researcher can decide the appropriate amount and dosage regimen. Dosage can vary, and can be administered in one or more dose administrations daily, weekly, monthly, or yearly, for one or several days.

As used herein, the terms “treatment,” “treating,” or “treat” refer to therapeutic treatment, in which the object is to inhibit or lessen an undesired physiological change or disorder or to promote a beneficial phenotype in a patient. For example, “treatment,” “treating,” or “treat” refer to clinical intervention in an attempt to alter the natural course of an individual's affliction, disease, or disorder. The terms include, for example, prophylaxis before or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration, or palliation of the disease state, and improved prognosis. In some embodiments, fusion proteins are used to control the cellular and clinical manifestations of kidney disorders, FSGS, IgA nephropathy, MCD, diabetic nephropathy, Alport syndrome, lupus nephritis, membranous nephropathy, acute kidney injury, Goodpasture syndrome, nephrotic syndrome, chronic proteinuria, chronic kidney disease, C3G, dense deposit disease, glomerulonephritis, membranoproliferative glomerulonephritis, polycystic kidney disease, hypertensive nephropathy, nephrosclerosis, aHUS, ischemia reperfusion injury, or rejection of a transplanted organ, such as a kidney. In some embodiments, the disease is FSGS.

A “variant” refers to a polynucleotide or a polypeptide that is substantially homologous to a native or reference polynucleotide or polypeptide. For example, a variant polynucleotide is substantially homologous to a native or reference polynucleotide but has a polynucleotide sequence different from that of the native or reference polynucleotide because of one or a plurality of deletions, insertions, and/or substitutions. In another example, a variant polypeptide is substantially homologous to a native or reference polypeptide but has an amino acid sequence different from that of the native or reference polypeptide because of one or a plurality of deletions, insertions, and/or substitutions. Variant polypeptide sequences encoding polynucleotide sequences encompass sequences that comprise one or more additions, deletions, or substitutions of nucleotides when compared to a native or reference polynucleotide sequence, that encode a variant protein or fragment thereof that retains activity. A wide variety of mutagenesis approaches are known in the art and can be applied by a person of ordinary skill in the art. A variant polynucleotide or polypeptide sequence can be at least 80%, at least 85%, at least at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a variant sequence can be determined, for example, by comparing the two sequences using freely available computer programs commonly employed for this purpose on the world wide web (e.g., BLASTp or BLASTn with default settings).

A “vector” as used herein refers to a macromolecule or association of macromolecules that comprises or associates with a polynucleotide and which can be used to mediate delivery of the polynucleotide to a cell, either in vitro or in vivo. Illustrative vectors include, for example, plasmids, viral vectors, liposomes, and other gene delivery vehicles.

### DETAILED DESCRIPTION

Described herein are complement alternative pathway-specific C3 and C5 convertase inhibitors that regulate complement alternative pathway activity. Described herein are a series of low molecular weight complement alternative pathway (CAP) activation and amplification loop-inhibiting molecules designed with mechanisms for binding to kidney epithelial cells. The compositions and methods described herein feature fusion proteins that include a fragment of complement factor H (FH) which may be fused to a VHH domain, a fragment of factor H-related protein 5 (FHRP5), and/or one or more kidney targeting motifs (e.g., one or more cyclic arginylglycylaspartic acid (RGD) motifs).

These fusion molecules include Complement Factor H (FH) catalytic domain short consensus repeats (SCRs) 1-4 for providing Factor I-mediated cofactor activity and decay acceleration functions through C3b binding. Additional, Factor H SCRs (e.g., SCR 5 and SCR 6) may be included to increase activity, stability, or structural flexibility. The fusion proteins, according to the disclosure herein, containing the FH catalytic domain SCRs may also include short amino acid sequence motifs or complement molecule domains which recognize integrin or damage markers present on injured kidney epithelial and tubular interstitial cell surfaces. Described herein are fusion proteins which may include a single-domain, variable heavy chain only (VHH) camelid antibody, to enable kidney epithelial cell deposition, improve expression, facilitate purification, and provide an exogenous probe for detection. Collectively, the use of these targeting residues, coupled with the inherent clearance kinetics of low molecular weight proteins, provide selective localization of CAP inhibitor fusion proteins to renal epithelial cells.

Diseases mediated by complement dysregulation are often a result of complement overactivity both in the fluid phase and at the cell surface. Described herein are compositions and methods for treating diseases mediated by complement dysregulation. Examples of disorders mediated by complement alternative pathway activation or dysregulation include, for example, kidney disorders, FSGS, IgA nephropathy, MCD, diabetic nephropathy, Alport syndrome, lupus nephritis, membranous nephropathy, acute kidney injury, Goodpasture syndrome, nephrotic syndrome, chronic proteinuria, chronic kidney disease, C3G, dense deposit disease, glomerulonephritis, membranoproliferative glomerulonephritis, polycystic kidney disease, hypertensive nephropathy, nephrosclerosis, aHUS, ischemia reperfusion injury, or rejection of a transplanted organ, such as a kidney. In some embodiments, the disease is FSGS.

The fusion protein or fusion proteins according to the disclosure herein regulate(s) complement alternative pathway activity, such as by irreversibly inactivating C3b and attenuating C3 and C5 convertase activity. The constructs target the complement alternative pathway and leave activation (protection) via classical and lectin pathways intact.

### Fusion Proteins

As described herein, fusion proteins of the disclosure include a fragment of factor H and may include an integrin recognition motif or a fragment of FHRP5. The constructs can be used as therapeutic agents to treat diseases mediated by complement alternative pathway activation or dysregulation (e.g., FSGS).

In humans, several regulatory proteins are encoded by a cluster of genes located on the long arm of chromosome 1. This region is called the regulator of complement activation (RCA) gene cluster. Although the proteins within the RCA family vary in size, they share primary amino acid structure similarities. The best studied members of the RCA family are factor H, FHL-1, CR1, DAF, MCP, and C4b-binding protein (C4BP). The members of this family are organized in tandem structural units, termed short consensus repeats (SCRs), which are present in multiple copies in the protein. Each SCR consists of ˜60-70 highly conserved amino acids, including four cysteine residues.

In some embodiments, the portion of the fusion protein suitable for inhibiting activity of the complement alternative pathway is fused with a VHH, for increased duration of effect.

In certain embodiments, the portion of the fusion protein suitable for inhibiting activity of the complement alternative pathway includes a fragment of factor H. The fragment of factor H may include at least the first four N-terminal SCR domains of factor H (e.g., SCRs 1, 2, 3, and 4). In certain embodiments, the fragment of factor H includes at least the first five N-terminal SCR domains of factor H (e.g., SCRs 1, 2, 3, 4, and 5); also known as the cofactor and decay accelerating domains. In certain embodiments, the fragment of factor H includes at least the first six N-terminal SCR domains of factor H (e.g., SCRs 1, 2, 3, 4, 5, and 6).

In some embodiments, the fragment of factor H may include a polypeptide sequence that is at least 85% (e.g., 86%, 87%, 88%, 89%, 90%, 91%, 92%, 93%, 94%, 95, 96%, 97%, 98%, or 99%) identical to SEQ ID NO: 24. In some embodiments, the fragment of factor H may include a polypeptide sequence that is at least 85% (e.g., 86%, 87%, 88%, 89%, 90%, 91%, 92%, 93%, 94%, 95, 96%, 97%, 98%, or 99%) identical to SEQ ID NO: 25. In some embodiments, the fragment of factor H may include a polypeptide sequence that is at least 85% (e.g., 86%, 87%, 88%, 89%, 90%, 91%, 92%, 93%, 94%, 95, 96%, 97%, 98%, or 99%) identical to SEQ ID NO: 26. In some embodiments, the fragment of factor H may include a polypeptide sequence that is at least 85% (e.g., 86%, 87%, 88%, 89%, 90%, 91%, 92%, 93%, 94%, 95, 96%, 97%, 98%, or 99%) identical to SEQ ID NO: 27. In some embodiments, the fragment of factor H may include a polypeptide sequence that is at least 85% (e.g., 86%, 87%, 88%, 89%, 90%, 91%, 92%, 93%, 94%, 95, 96%, 97%, 98%, or 99%) identical to SEQ ID NO: 28. In some embodiments, the fragment of factor H may include a polypeptide sequence that is at least 85% (e.g., 86%, 87%, 88%, 89%, 90%, 91%, 92%, 93%, 94%, 95, 96%, 97%, 98%, or 99%) identical to SEQ ID NO: 29. In some embodiments, the fragment of factor H may include a polypeptide sequence that is at least 85% (e.g., 86%, 87%, 88%, 89%, 90%, 91%, 92%, 93%, 94%, 95, 96%, 97%, 98%, or 99%) identical to SEQ ID NO: 16. In some embodiments, the fragment of factor H may include a polypeptide sequence that is at least 85% (e.g., 86%, 87%, 88%, 89%, 90%, 91%, 92%, 93%, 94%, 95, 96%, 97%, 98%, or 99%) identical to SEQ ID NO: 17. In some embodiments, the fragment of factor H may include a polypeptide sequence that is at least 85% (e.g., 86%, 87%, 88%, 89%, 90%, 91%, 92%, 93%, 94%, 95, 96%, 97%, 98%, or 99%) identical to SEQ ID NO: 18.

The fusion protein may include, in addition to a fragment of factor H, an integrin binding domain. The fragment of factor H in the fusion protein may include at least the first four, five, or six N-terminal SCR domains of factor H and the integrin binding domain may include an arginylglycylaspartic acid (RGD) peptide motif. The arginylglycylaspartic acid peptide motif may include a cyclo(RGD)₄ peptide (SEQ ID NO: 21). In some embodiments, the fusion protein may include an integrin binding domain which includes a polypeptide sequence that is at least 85% (e.g., 86%, 87%, 88%, 89%, 90%, 91%, 92%, 93%, 94%, 95, 96%, 97%, 98%, or 99%) identical to SEQ ID NO: 21.

In certain embodiments, the fragment of factor H includes at least the first five N-terminal SCR domains of factor H (e.g., SCRs 1, 2, 3, 4, and 5) and the integrin binding domain includes a cyclo(RGD)₄ peptide. In certain embodiments, the fragment of factor H includes at least the six five N-terminal SCR domains of factor H (e.g., SCRs 1, 2, 3, 4, 5 and 6), and the integrin binding domain includes a cyclo(RGD)₄ peptide.

The fusion protein may include, in addition to a fragment of factor H, a fragment of a factor H-related protein 5 (FHRP5). In some embodiments, the fusion protein may include a fragment of a FHRP5 domain which includes a polypeptide sequence that is at least 85% (e.g., 86%, 87%, 88%, 89%, 90%, 91%, 92%, 93%, 94%, 95, 96%, 97%, 98%, or 99%) identical to SEQ ID NO: 22. In some embodiments, the fusion protein may include a fragment of a FHRP5 domain which includes a polypeptide sequence of SEQ ID NO: 22. The fragment of factor H in the fusion protein may include at least the first four, five, or six N-terminal SCR domains of factor H, and the fragment of FHRP5 in the fusion protein may include at least the seventh and/or eighth N-terminal SCR domains of FHRP5.

In certain embodiments, the fragment of factor H includes at least the first five N-terminal SCR domains of factor H (e.g., SCRs 1, 2, 3, 4, and 5) and the fragment FHRP5 includes at least the seventh and eighth N-terminal SCR domains of FHRP5.

In some embodiments, the fragment of the factor H portion of the fusion protein is a functional fragment of wild-type factor H. In some embodiments, the factor H, or fragment thereof, portion of the fusion protein is derived from a substituted (e.g., conservatively substituted) factor H or an engineered factor H (e.g., a factor H engineered to increase stability, activity, and/or other desirable properties of the protein, as determined by a predictive model or assay known to one of skill in the art, such as described herein).

In some embodiments, the fragment of the FHRP5 portion of the fusion protein is a functional fragment of wild-type FHRP5. In some embodiments, the FHRP5, or fragment thereof, portion of the fusion protein composition is derived from a substituted (e.g., conservatively substituted) FHRP5 or an engineered FHRP5 (e.g., a FHRP5 engineered to increase stability, activity, and/or other desirable properties of the protein, as determined by a predictive model or assay known to one of skill in the art, such as an assay described herein).

Amino acid substitutions can be introduced into the fusion proteins described herein to improve functionality. For example, amino acid substitutions can be introduced into the fragment of factor H, the integrin binding domain, or the fragment of FHRP5, wherein an amino acid substitution increases binding affinity of the fragment of factor H, the integrin binding domain, or the fragment of FHRP5 for its ligand(s). Similarly, amino acid substitutions can be introduced into the fragment of factor H, or fragment thereof, to increase functionality and/or to improve the pharmacokinetics of the fusion protein.

In certain embodiments, the fusion proteins described herein can be fused with another compound, such as a compound to increase the half-life of the polypeptide and/or to reduce potential immunogenicity of the fusion protein (for example, polyethylene glycol (PEG)). PEG can be used to improve water solubility, reduce the rate of kidney clearance, and reduce immunogenicity of the fusion protein (see, e.g., U.S. Pat. No. 6,214,966, the disclosure of which is incorporated herein by reference). The fusion proteins described herein can be PEGylated by any means known to one skilled in the art.

The fragment of factor H may be prepared by a number of synthetic methods of peptide synthesis by fragment condensation of one or more amino acid residues, according to conventional peptide synthesis methods known in the art (Amblard, M. et al., Mol. Biotechnol., 33:239-54, 2006).

Alternatively, a fragment of factor H, integrin binding domain, and/or fragment of FHRP5 may be produced by expression in a suitable prokaryotic or eukaryotic system. In some embodiments, a DNA construct may be inserted into a plasmid vector adapted for expression in a suitable host cell (such as E. coli) or a yeast cell (such as S. cerevisiae or P. pastoris), or into a baculovirus vector for expression in an insect cell, or a viral vector for expression in a mammalian cell. Examples of suitable mammalian cells for recombinant expression include, e.g., a human embryonic kidney cell (HEK) (e.g., HEK 293), a Chinese Hamster Ovary (CHO) cell, L cell, C127 cell, 3T3 cell, BHK cell, or COS-7 cell. Suitable expression vectors include the regulatory elements necessary and sufficient for expression of the DNA in the host cell. In some embodiments, a leader or secretory sequence or a sequence that is employed for purification of the fusion protein (e.g., a histidine tag), can be included in the fusion protein. The fragment of factor H, integrin binding domain, and/or fragment of FHRP5 produced by gene expression in a recombinant prokaryotic or eukaryotic system may be purified according to methods known in the art (See, e.g., Structural Genomics Consortium, Nat. Methods, 5:135-46, 2008).

In certain embodiments, the cyclized integrin binding domain and fragments of FHRP5 are also produced by the same methods described for the expression and purification of fragments of factor H.

In some embodiments, the fusion protein has the structure, from N-terminus to C-terminus, of Formula I:

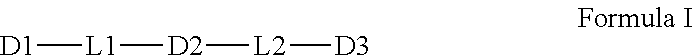

wherein:
D1 is a fragment of FH (e.g., a fragment of FH with the amino acid sequence of any one of SEQ ID NOS: 16-18 or a variant thereof with 85% sequence identity thereto or greater);
L1 is absent (e.g., L1 is a covalent bond between D1 and D2 or between D1 and D3), or is a linker with an amino acid sequence of at least one amino acid (e.g., the linker may have the amino acid sequence of any one of SEQ ID NOs 30-122 or a variant thereof with 85% sequence identity thereto or greater) between D1 and D2 or between D1 and D3;
D2 is absent (e.g., D2 is a covalent bond between L1 and D3, between D1 and L2, or between L1 and L2) or is a VHH domain, such as a single domain antibody (e.g., camelid single-domain antibody VHH with the amino acid sequence of any one of SEQ ID NOs: 19-20 and 23 or a variant thereof with 85% sequence identity thereto or greater);
L2 is absent (e.g., L2 is a covalent bond between D2 and D3), or is a linker with an amino acid sequence of at least one amino acid (e.g., the linker may have the amino acid sequence of any one of SEQ ID NOs: 30-122 or a variant thereof with 85% sequence identity thereto or greater) between D2 and D3; and
D3 is an integrin recognition domain (e.g., an arginylglycylaspartic acid (RGD) peptide motif e.g., cyclo(RGD)₄ with the amino acid sequence of SEQ ID NO: 21 or a variant thereof with 85% sequence identity thereto or greater).

In some embodiments, the fragment of FH of D1 includes one or more FH SCR domains, optionally wherein the one of more SCR domains are selected from the group consisting of SCR 1, 2, 3, 4, 5, or 6 or a variant thereof with at least 85% (e.g., 87%, 90%, 95%, 97%, or 99%) sequence identity to any one of SEQ ID NOS: 24-29. In some embodiments, the FH SCR domains are selected from the group consisting of SCR [1-5] or a variant thereof with at least 85% (e.g., 87%, 90%, 95%, 97%, or 99%) sequence identity to either of SEQ ID NOs: 16 or 17, or SCR [1-6] or a variant thereof with at least 85% (e.g., 87%, 90%, 95%, 97%, or 99%) sequence identity to SEQ ID NO: 18.

L1 and L2 may be linkers of the same type and/or sequence or of a different type and/or sequence.

In some embodiments, the composition of Formula I includes the amino acid sequence of any one of SEQ ID NOs: 4, 5, 8, 9, and 13-15 and variants thereof with at least 85%, 87%, 90%, 95%, 97%, or 99% sequence identity thereto. In some embodiments, the composition of Formula I is encoded by the nucleic acid sequence of any one of SEQ ID NOS: 128, 129, 132, 133, and 137-139 and variants thereof with at least 85%, 87%, 90%, 95%, 97%, or 99% sequence identity thereto.

In some embodiments, the fusion protein has the structure, from N-terminus to C-terminus, of Formula II:

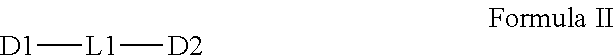

wherein:
D1 is a fragment of FH (e.g., a fragment of FH with the amino acid sequence of any one of SEQ ID NO: 16-18 or a variant thereof with 85% sequence identity thereto or greater.
L1 is absent (e.g., L1 is a covalent bond between D1 and D2), or is a linker with an amino acid sequence of at least one amino acid (e.g., the linker may have the amino acid sequence of any one of SEQ ID NOs 30-122 or a variant thereof with 85% sequence identity thereto or greater) between D1 and D2; and
D2 is a fragment of a factor H-related protein 5 (FHRP5) (e.g., a fragment of FHRP5 with the amino acid sequence of SEQ ID NO: 22 or a variant thereof with 85% sequence identity thereto or greater).

In some embodiments, the fragment of FH of D1 includes one or more FH SCR domains, optionally wherein the one or more SCR domains are selected from the group consisting of SCR 1, 2, 3, 4, 5, or 6 or a variant thereof with at least 85% (e.g., 87%, 90%, 95%, 97%, or 99%) sequence identity to any one of SEQ ID NOS: 24-29. In some embodiments, the FH SCR domains are selected from the group consisting of SCR [1-5] or a variant thereof with at least 85% (e.g., 87%, 90%, 95%, 97%, or 99%) sequence identity to either of SEQ ID NO:16 or 17, or SCR [1-6] or a variant thereof with at least 85% (e.g., 87%, 90%, 95%, 97%, or 99%) sequence identity to SEQ ID NO: 18. In some embodiments, the fragment of FHRP5 includes one or more FHRP5 domains, optionally wherein the domains are selected from domains 7 and 8 (e.g., the amino acid sequence of SEQ ID NO: 22). In some embodiments, the fragment of FHRP5 includes domains 7-8 or a variant thereof with at least 85% (e.g., 87%, 90%, 95%, 97%, or 99%) sequence identity to SEQ ID NO: 22.

In some embodiments, the composition of Formula II includes either SEQ ID NO: 6 or 10 or variants thereof with at least 85%, 87%, 90%, 95%, 97%, or 99% sequence identity thereto. In some embodiments, the composition of Formula II is encoded by the nucleic acid sequence of SEQ ID NO: 130 or 134 or variants thereof with at least 85%, 87%, 90%, 95%, 97%, or 99% sequence identity thereto.

In some embodiments, the fusion protein has the structure, from N-terminus to C-terminus, of Formula III:

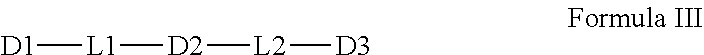

wherein:
D1 is an integrin recognition domain (e.g., an arginylglycylaspartic acid (RGD) peptide motif e.g., cyclo(RGD)₄ of SEQ ID NO: 21 or a variant thereof with 85% sequence identity thereto or greater);
L1 is absent (e.g., L1 is a covalent bond between D1 and D2 or between D1 and D3), or is a linker with an amino acid sequence of at least one amino acid (e.g., the linker may have the amino acid sequence of any one of SEQ ID NOs 30-122 or a variant thereof with 85% sequence identity thereto or greater) between D1 and D2 or between D1 and D3;
D2 is absent (e.g., D2 is a covalent bond between L1 and D3, between D1 and L2, or between L1 and L2) or is a VHH domain, such as a single domain antibody (e.g., camelid single-domain antibody VHH with the amino acid sequence of any one of SEQ ID NOS: 19-20 and 23 or a variant thereof with 85% sequence identity thereto or greater);
L2 is absent (e.g., L2 is a covalent bond between D2 and D3), or is a linker with an amino acid sequence of at least one amino acid (e.g., the linker may have the amino acid sequence of any one of SEQ ID NOs: 30-122 or a variant thereof with 85% sequence identity thereto or greater) between D2 and D3; and
D3 is a fragment of FH (e.g., a fragment of FH with the amino acid sequence of any one of SEQ ID NOS: 16-18 or a variant thereof with 85% sequence identity thereto or greater).

In some embodiments, the fragment of FH of D3 includes one or more FH SCR domains, optionally wherein the one of more SCR domains are selected from the group consisting of SCR 1, 2, 3, 4, 5, or 6 or a variant thereof with at least 85% (e.g., 87%, 90%, 95%, 97%, or 99%) sequence identity to any one of SEQ ID NOS: 24-29. In some embodiments, the FH SCR domains are selected from the group consisting of SCR [1-5] or a variant thereof with at least 85% (e.g., 87%, 90%, 95%, 97%, or 99%) sequence identity to either SEQ ID NO: 16 or 17, or SCR [1-6] or a variant thereof with at least 85% (e.g., 87%, 90%, 95%, 97%, or 99%) sequence identity to SEQ ID NO: 18.
L1 and L2 may be linkers of the same type and/or sequence or of a different type and/or sequence.

In some embodiments, the composition of Formula III includes either SEQ ID NO: 2 or 3 or a variant thereof with at least 85% (e.g., 87%, 90%, 95%, 97%, or 99%) sequence identity thereto. In some embodiments, the composition of Formula III is encoded by the nucleic acid sequence of either SEQ ID NO: 126 or 127 or variants thereof with at least 85%, 87%, 90%, 95%, 97%, or 99% sequence identity thereto.

In some embodiments, the fusion protein has the structure, from N-terminus to C-terminus, of Formula IV:

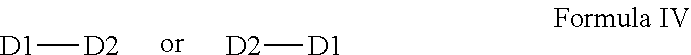

wherein:
D1 is a VHH domain, such as a single domain antibody (e.g., camelid single-domain antibody VHH with the amino acid sequence of any one of SEQ ID NOS: 19-20 and 23 or a variant thereof with 85% sequence identity thereto or greater);
D2 is a fragment of FH (e.g., a fragment of FH of either SEQ ID NO: 16 or 17 or a variant thereof with 85% sequence identity thereto or greater).

In some embodiments, the fragment of FH of D3 includes one or more FH SCR domains, optionally wherein the one of more SCR domains are selected from the group consisting of SCR 1, 2, 3, 4, 5, or 6 or a variant thereof with 85% sequence identity thereto or greater. In some embodiments, the FH SCR domains are selected from the group consisting of SCR [1-5] or a variant thereof with at least 85% (e.g., 87%, 90%, 95%, 97%, or 99%) sequence identity to SEQ ID NO: 16 or 17, or SCR [1-6] or a variant thereof with at least 85% (e.g., 87%, 90%, 95%, 97%, or 99%) sequence identity to SEQ ID NO: 18.

In some embodiments, the composition of Formula IV includes any one of SEQ ID NOs:1, 7, 11, and 12 or a variant thereof with at least 85% (e.g., 87%, 90%, 95%, 97%, or 99%) sequence identity thereto. In some embodiments, the composition of Formula IV is encoded by the nucleic acid sequence of any one of SEQ ID NOs: 125, 131, 135, and 136 or variants thereof with at least 85%, 87%, 90%, 95%, 97%, or 99% sequence identity thereto.

### Immunoglobin Proteins and Domains

The fusion proteins described herein may contain a single chain VHH domain. Such antibodies exist naturally in camelids and sharks (Saerens et al., Curr. Opin. Pharmacol., 8:600-608, 2008). Camelid antibodies are described in, for example, U.S. Pat. Nos. 5,759,808; 5,800,988; 5,840,526; 5,874,541; 6,005,079; and 6,015,695, the entire contents of each are incorporated herein by reference.

Exemplary VHH domains include those having the sequence of

(SEQ ID NO: 19)
QVQLVESGGGLVKPGGSLRLSCAASGRTFSSYAMGWFRQAPGKEREFVSA
ISGSGGSTYYADSVKGRFTISRDNAKNSLYLQMNSLRAEDTAVYYCAADL
GDGSWVDYVNAEPYEYDYWGQGTLVTVSS,
(SEQ ID NO: 20)
EVQLVESGGGLVKPGGSLRLSCAASGRTFSSYAMG WFRQAPGKEREFVS
AISGSGGSTYYADSVKGRFTISRDNAKNSLYLQMNSLRAEDTAVYYCAAD
LGDGSWVDYVNAEPYEYDYWGQGTLVTVSS,
or
(SEQ ID NO: 23)
EVQLLESGGGLVQPGGSLRLSCAASGRTFSSYAMGWFRQAPGKEREFVSAI
SGSGGSTYYADSVKGRFTISRDNSKNTLYLQMNSLKPEDTAVYYCAADLGD
GSWVDYVNMEPYEYDYWGQGTQVTVSS.

In some embodiments, the fusion protein may include a VHH domain which includes a polypeptide sequence that is at least 85% (e.g., 86%, 87%, 88%, 89%, 90%, 91%, 92%, 93%, 94%, 95, 96%, 97%, 98%, or 99%) identical to SEQ ID NO: 19. In some embodiments, the fusion protein may include a VHH domain which includes a polypeptide sequence that is at least 85% (e.g., 86%, 87%, 88%, 89%, 90%, 91%, 92%, 93%, 94%, 95, 96%, 97%, 98%, or 99%) identical to SEQ ID NO: 20. In some embodiments, the fusion protein may include a VHH domain which includes a polypeptide sequence that is at least 85% (e.g., 86%, 87%, 88%, 89%, 90%, 91%, 92%, 93%, 94%, 95, 96%, 97%, 98%, or 99%) identical to SEQ ID NO: 23.

The fusion protein may include, from N-terminus to C-terminus, D1-L1-D2-L2-D3, in which D1 includes a fragment of an FH protein, such as FH SCR 1-5 or FH SCR 1-6, L1 is absent or includes a linker, D2 includes a VHH domain, L2 is absent or includes a linker, and D3 includes an integrin recognition domain, such as cyclo(RGD)₄.

In another example, the fusion protein may include, from N-terminus to C-terminus, D1-L1-D2-L2-D3, in which D1 includes an integrin recognition domain, such as cyclo(RGD)₄, L1 is absent or include a linker, D2 includes a VHH domain, L2 includes a linker or is absent, and D3 includes a fragment of an FH protein, such as FH SCR 1-5.

In another example, the fusion protein may include, from N-terminus to C-terminus, D1-D2 or D2-D1, in which D1 includes a VHH domain and D2 includes a fragment of an FH protein. The fusion protein may have the amino acid sequence of any one of SEQ ID NOs: 1, 7, 11, and 12 or a variant thereof with at least 85% (e.g., 87%, 90%, 95%, 97%, or 99%) sequence identity to any one of SEQ ID NOs: 1, 7, 11, and 12.

In some embodiments, the factor H fusion protein including a VHH domain has an increased intrarenal residence time along the kidney epithelial surface relative to a fusion protein lacking the VHH domain. Without being bound to a particular theory, the size of the fusion proteins described herein (e.g., about ≤60 kDa (such as less than 60 kDa) is believed to enable the fusion proteins to gain entry to extravascular compartments within the kidney inaccessible by monoclonal antibodies and albumin-bond bispecifics, and the use of a VHH domain in the fusion proteins described herein is believed to enable the fusion proteins to deposit on the apical membrane of proximal tubule and parietal epithelial cells, where naturally low levels of membrane-associated surface regulators confer susceptibility to CAP products, and to exhibit extended residence along the kidney epithelium. In some embodiments, the intrarenal residence time is increased by at least 1 fold (e.g., 2 fold, 3 fold, 4 fold, 5 fold, 6 fold, 7 fold, 8 fold, 9 fold, or 10 fold) relative to a fusion protein lacking the VHH domain. In some embodiments, the intrarenal residence time is between 24 hours and 96 hours (e.g., between 36 hours and 96 hours, 48 hours and 96 hours, 60 hours and 96 hours, 72 hours and 96 hours, and 60 hours and 84 hours).

### Integrin Binding Domain

The fusion protein may also have an integrin binding domain, which may act as a targeting motif to improve the pharmacokinetics of the fusion protein and mediate renal cell-specific targeting at sites of damage or remodeling. The integrin binding domain may be added as an additional domain to any one of the fusion proteins described herein.

Exemplary integrin binding domains include one or more cyclic arginylglycylaspartic acid (RGD) peptide motifs fused to either the N- or C-terminus of the fusion protein. RGD motifs engage the extracellular domains of integrin α- and β-subunits on the cell surface that can be upregulated in response to injury (e.g., including renal fibrosis mediated by TGF-β signaling). Without being bound to a particular theory, it is expected that the inclusion of a cyclic RGD motif may also limit pro-TGF-β ligand binding and prevent pro-fibrotic signaling. Different variants of integrin binding motifs can be constructed and attached to the fusion protein. In some embodiments, the fusion protein may include an integrin binding domain which includes an amino sequence that is at least 85% (e.g., 86%, 87%, 88%, 89%, 90%, 91%, 92%, 93%, 94%, 95, 96%, 97%, 98%, or 99%) identical to SEQ ID NO: 21.

The fusion protein may include, from N-terminus to C-terminus, D1-L1-D2-L2-D3, in which D1 includes a fragment of an FH protein, such as an FH with SCR [1-5] or an FH with SCR [1-6]. The fusion protein may have the amino acid sequence of any one of SEQ ID NOs: 4, 5, 8, 9, and 13-15 or variant thereof with at least 85% (e.g., 87%, 90%, 95%, 97%, or 99%) sequence identity to any one of SEQ ID NOS: 4, 5, 8, 9, and 13-15. The fusion protein may also have a fragment of a FH protein, such as an FH with SCR [1-5] or an FH with SCR [1-6]. The fusion protein may have the amino acid sequence of either of SEQ ID NO: 2 or 3 or a variant thereof with at least 85% (e.g., 87%, 90%, 95%, 97%, or 99%) sequence identity to either SEQ ID NO: 2 or 3.

### Linkers for the Fusion Proteins

The L1 and L2 domains of the fusion proteins described herein are linkers. A linker is used to create a linkage or connection between, for example, polypeptides, or protein domains. For example, a fragment of factor H may be linked directly to a VHH domain (e.g., single-domain camelid VHH domain) by one or more suitable linkers. A linker can be a simple covalent bond, e.g., a peptide bond, a synthetic polymer, e.g., a PEG polymer, or any kind of bond created from a chemical reaction, e.g., chemical conjugation. The peptide linker can be, for example, a linker of one or more amino acid residues inserted or included at the transition between the two domains (e.g., a fragment of the FH protein and a VHH domain). The identity and sequence of amino acid residues in the linker may vary depending on the desired secondary structure. For example, glycine, serine, and alanine are useful for linkers given their flexibility. Any amino acid residue can be considered as a linker in combination with one or more other amino acid residues, which may be the same as or different from the first amino acid residue, to construct larger peptide linkers as necessary depending on the desired length and/or properties.

A variety of linkers can be used to fuse two or more protein domains together (e.g., a fragment of factor H and a VHH domain). Linkers may be flexible, rigid, or cleavable. Linkers may be structured or unstructured. The residues for the linker may be selected from naturally occurring amino acids, non-naturally occurring amino acids, and modified amino acids. The linker may include at least 1 or more, 2 or more, 5 or more, 10 or more, 15 or more, or 20 or more amino acid residues. Peptide linkers can include, but are not limited to, glycine linkers, glycine-rich linkers, serine-glycine linkers, and the like. A glycine-rich linker includes at least about 50% glycine.

In some embodiments, the linker(s) used confers one or more other favorable properties or functionality to the polypeptide(s) described herein, and/or provides one or more sites for the formation of derivatives and/or for the attachment of functional groups. For example, linkers containing one or more charged amino acid residues can provide improved hydrophilic properties, whereas linkers that form or contain small epitopes or tags can be used for the purposes of detection, identification, and/or purification. A skilled artisan will be able to determine the optimal linkers for use in a specific polypeptide.

When two or more linkers are used for a polypeptide, the linkers may be the same or different.

Linkers can contain motifs, e.g., multiple or repeating motifs. In one embodiment, the linker has the amino acid sequence GS, or repeats thereof (Huston, J. et al., Methods Enzymol., 203:46-88, 1991). In another embodiment, the linker includes the amino acid sequence EK, or repeats thereof (Whitlow, M. et al., Protein Eng., 6:989-95, 1993). In another embodiment, the linker includes the amino acid sequence GGS, or repeats thereof.

In another embodiment, the linker includes the amino acid sequence GGGGA (SEQ ID NO: 80) or repeats thereof. In certain embodiments, the linker contains more than one repeat of GGS or GGGGS (U.S. Pat. No. 6,541,219, the entire contents of which are herein incorporated by reference). In one embodiment, the peptide linker may be rich in small or polar amino acids, such as G and S, but can contain additional amino acids, such as T and A, to maintain flexibility, as well as polar amino acids, such as K and E, to improve solubility.

Exemplary linkers include, but are not limited to: G₄S (SEQ ID NO: 36), (G₄A)₂G₄S (SEQ ID NO: 34), (G₄A)₂G₃AG₄S (SEQ ID NO: 30), G₄AG₃AG₄S (SEQ ID NO: 33), G₄SDA (SEQ ID NO: 79), G₄SDAA (SEQ ID NO: 31), G₄S (SEQ ID NO: 36), (G₄S)₂ (SEQ ID NO: 37), (G₄S)₃ (SEQ ID NO: 35), (G₄S)₄ (SEQ ID NO: 39), (G₄S)₅ (SEQ ID NO: 40), (G₄S)₆ (SEQ ID NO: 41), EAAAK (SEQ ID NO: 95), (EAAAK)₃ (SEQ ID NO: 42), PAPAP (SEQ ID NO: 43), G₄SPAPAP (SEQ ID NO: 44), PAPAPG₄S (SEQ ID NO: 45), GSTSGKSSEGKG (SEQ ID NO: 46), (GGGDS)₂ (SEQ ID NO: 47), (GGGES)₂ (SEQ ID NO: 48), GGGDSGGGGS (SEQ ID NO: 49), GGGASGGGGS (SEQ ID NO: 50), GGGESGGGGS (SEQ ID NO: 51), ASTKGP (SEQ ID NO: 52), ASTKGPSVFPLAP (SEQ ID NO: 53), G₃P (SEQ ID NO: 54), G₇P (SEQ ID NO: 55), PAPNLLGGP (SEQ ID NO: 56), G₆ (SEQ ID NO: 57), G₁₂ (SEQ ID NO: 58), APELPGGP (SEQ ID NO: 59), SEPQPQPG (SEQ ID NO: 60), (G₃S₂)₃ (SEQ ID NO: 61), GGGGGGGGGGGGS (SEQ ID NO: 62), GGGGSGGGGGGGGGS (SEQ ID NO: 63), (GGSSS)₃ (SEQ ID NO: 64), (GS₄)₃ (SEQ ID NO: 65), G₄A(G₄S)₂ (SEQ ID NO: 66), G₄SG₄AG₄S (SEQ ID NO: 67), G₃AS(G₄S)₂ (SEQ ID NO: 68), G₄SG₃ASG₄S (SEQ ID NO: 69), G₄SAG₃SG₄S (SEQ ID NO: 70), (G₄S)₂AG₃S (SEQ ID NO: 71), G₄SAG₃SAG₃S (SEQ ID NO: 72), G₄D(G₄S)₂ (SEQ ID NO: 73), G₄SG₄DG₄S (SEQ ID NO: 74), (G₄D)₂G₄S (SEQ ID NO: 75), G₄E(G₄S)₂ (SEQ ID NO: 76), G₄SG₄EG₄S (SEQ ID NO: 77), and (G₄E)₂G₄S (SEQ ID NO: 78), (GGGGS)ₙ, wherein n can be any number, KESGSVSSEQLAQFRSLD (SEQ ID NO: 82), and EGKSSGSGSESKST (SEQ ID NO: 83), (Gly)₈ (SEQ ID NO: 84), GSAGSAAGSGEF (SEQ ID NO: 87), and (Gly)₆ (SEQ ID NO: 57). Exemplary rigid linkers include but are not limited to A(EAAAK)A (SEQ ID NO: 86), A(EAAAK)ₙA, wherein n can be any number, or (XP)ₙ wherein n can be any number, with X designating any amino acid. Exemplary in vivo cleavable linkers include, for example, LEAGCKNFFPRSFTSCGSLE (SEQ ID NO: 87), GSST (SEQ ID NO: 88), and CRRRRRREAEAC (SEQ ID NO: 89). In some embodiments, a linker can contain 2 to 12 amino acids including motifs of GS, e.g., GS, GSGS (SEQ ID NO: 90), GSGSGS (SEQ ID NO: 91), GSGSGSGS (SEQ ID NO: 92), GSGSGSGSGS (SEQ ID NO: 93), or GSGSGSGSGSGS (SEQ ID NO: 95). In certain other embodiments, a linker can contain 3 to 12 amino acids including motifs of GGS, e.g., GGS, GGSGGS (SEQ ID NO: 96), GGSGGSGGS (SEQ ID NO: 97), and GGSGGSGGSGGS (SEQ ID NO: 98). In yet other embodiments, a linker can contain 4 to 12 amino acids including motifs of GGSG, e.g., GGSG (SEQ ID NO: 99), GGSGGGSG (SEQ ID NO: 100), or GGSGGGSGGGSG (SEQ ID NO: 101). In other embodiments, a linker can contain motifs of GGGGS (SEQ ID NO: 36). In other embodiments, a linker can also contain amino acids other than glycine and serine, e.g., GENLYFQSGG (SEQ ID NO: 102), SACYCELS (SEQ ID NO: 103), RSIAT (SEQ ID NO: 104), RPACKIPNDLKQKVMNH (SEQ ID NO: 105), GGSAGGSGSGSSGGSSGASGTGTAGGTGSGSGT GSG (SEQ ID NO: 16), AAANSSIDLISVPVDSR (SEQ ID NO: 107), GGSGGGSEGGGSEGGGSEGGGSEGGGSEGGGSGGGS (SEQ ID NO: 108), GGGGAGGGGAGGGGS (SEQ ID NO: 32), GGGGAGGGGAGGGGAGGGGS (SEQ ID NO: 110), DAAGGGGSGGGGSGGGGSGGGGSGGGGS (SEQ ID NO: 111), GGGGAGGGGAGGGGA (SEQ ID NO: 81), GGGGAGGGGAGGGAGGGGS (SEQ ID NO: 30), or GGSSRSSSSGGGGAGGGG (SEQ ID NO: 112).

In one embodiment, the linker is a cleavable linker, such as an enzymatically cleavable linker. Inclusion of a cleavable linker can aid in detection of the fusion protein. An enzymatically cleavable linker can be cleavable, for example, by trypsin, Human Rhinovirus 3C Protease (3C), enterokinase (Ekt), Factor Xa (FXa), Tobacco Etch Virus protease (TEV), or thrombin (Thr). Cleavage sequences for each of these enzymes are well known in the art. For example, trypsin cleaves peptides on the C-terminal side of lysine and arginine amino acid residues. If a proline residue is on the carboxyl side of the cleavage site, the cleavage will not occur. If an acidic residue is on either side of the cleavage site, the rate of hydrolysis has been shown to be slower. The following linkers are examples of linkers that can be cleaved using trypsin: K(G₄A)₂G₃AG₄SK, R(G₄A)₂G₃AG₄SR, K(G₄A)₂G₃AG₄SR, R(G₄A)₂G₃AG₄SK, K(G₄A)₂G₄SK, K(G₄A)₂G₄SR, R(G₄A)₂G₄SK, and R(G₄A)₂G₄SR.

An example of a protease cleavage site that can be included in an enzymatically cleavable linker is a tobacco etch virus (TEV) protease cleavage site, e.g., ENLYTQS, where the protease cleaves between the glutamine and the serine. Another example of a protease cleavage site that can be included in an enzymatically cleavable linker is an enterokinase cleavage site, e.g., DDDDK, where cleavage occurs after the lysine residue. Another example of a protease cleavage site that can be included in an enzymatically cleavable linker is a thrombin cleavage site, e.g., LVPR. For Human Rhinovirus 3C Protease, the cleavage site is LEVLFQGP where cleavage occurs between the glutamine and glycine residues. A cleavage site for Factor Xa protease is IEDGR, where cleavage occurs between the glutamic acid and aspartic acid residues.

The inclusion of the cleavable linker is useful in that it has a sequence of amino acids that is unique from other peptides in the human proteome that are generated with the above-mentioned enzymes. As such this excised linker may serve as a unique identifying peptide of the fusion protein when administered as a pharmaceutical preparation to humans. In this way, the cleavable linker may be detected and quantitated by mass spectrometry and be used to monitor the pharmacokinetics of the fusion protein.

In another embodiment, the linker is a polymeric or oligomeric glycine linker, and can include a lysine at the N-terminus, the C-terminus, or both the N- and the C-termini.

With reference to Formulas I and III above, the C-terminus of D1 may be linked to the N-terminus of D2. In a certain embodiment, the C-terminus of the FH fragment is linked to the N-terminus of a VHH. In certain embodiments, the C-terminus of an integrin binding domain is linked to the N-terminus of a VHH. In a certain embodiment, the C-terminus of D2 may be linked to the N-terminus of D3. In a certain embodiment, the C-terminus of a VHH may be linked to the N-terminus of an integrin binding domain. In a certain embodiment, the C-terminus of the VHH may be linked to the N-terminus of the FH fragment. In another example, the C-terminus of D1 may be linked to the N-terminus of D3. In certain embodiments, the C-terminus of the FH fragment is linked to the N-terminus of the integrin binding domain. In a certain embodiment, the C-terminus of the integrin binding domain is linked to the N-terminus of the FH fragment. In another example, the C-terminus of D2 may be linked to the N-terminus of D3. In certain embodiments, the C-terminus of the VHH may be linked to the N-terminus of the integrin binding domain. In certain embodiments, the C-terminus of the VHH may be linked to the N-terminus of the FH fragment.

With reference to Formula II above, the C-terminus of D1 may be linked to the N-terminus of D2. In a certain embodiment, the C-terminus of the FH fragment is linked to the N-terminus of the FHRP5 fragment.

TABLE 1
Fusion Proteins with the sequence, from N-
terminus to C-terminus, of D1-L1-D2-L2-D3

| | | | | | | Amino | Nucleic |
|---|---|---|---|---|---|---|---|
| Compound | | | | | | Acid | Acid |
| Name | D1 | L1 | D2 | L2 | D3 | Sequence | Sequence |
| Compound | hFH1-5 | G₄A | — | — | Cyclo(RGD)₄ | SEQ ID | SEQ ID |
| D | (SEQ ID | | | | (SEQ ID | NO: 4 | NO: 128 |
| | NO: 16) | | | | NO: 21) | | |
| Compound | hFH1-5 | — | VHH | G₄A | Cyclo(RGD)₄ | SEQ ID | SEQ ID |
| E | (SEQ ID | | (SEQ ID | | (SEQ ID | NO: 5 | NO: 129 |
| | NO: 16) | | NO: 19) | | NO: 21) | | |
| Compound | hFH1-5 | G₄A | — | — | Cyclo(RGD)₄ | SEQ ID | SEQ ID |
| H | (SEQ ID | | | | (SEQ ID | NO: 8 | NO: 132 |
| | NO: 17) | | | | NO: 21) | | |
| Compound | hFH1-5 | — | VHH | G₄A | Cyclo(RGD)₄ | SEQ ID | SEQ ID |
| I | (SEQ ID | | (SEQ ID | | (SEQ ID | NO: 9 | NO: 133 |
| | NO: 17) | | NO: 19) | | NO: 21) | | |
| Compound | hFH1-5 | — | VHH | G₄A | Cyclo(RGD)₄ | SEQ ID | SEQ ID |
| M | (SEQ ID | | (SEQ ID | | (SEQ ID | NO: 13 | NO: 137 |
| | NO: 16) | | NO: 20) | | NO: 21) | | |
| Compound | hFH1-6 | — | VHH | G₄A | Cyclo(RGD)₄ | SEQ ID | SEQ ID |
| N | (SEQ ID | | (SEQ ID | | (SEQ ID | NO: 14 | NO: 138 |
| | NO: 18) | | NO: 20) | | NO: 21) | | |
| Compound | hFH1-5 | G₄A | VHH | G₄A | Cyclo(RGD)₄ | SEQ ID | SEQ ID |
| O | (SEQ ID | | (SEQ ID | | (SEQ ID | NO: 15 | NO: 139 |
| | NO: 16) | | NO: 20) | | NO: 21) | | |

“—” indicates the absence of a feature.

TABLE 2
Fusion Proteins with the sequence, from N-terminus to C-terminus, of D1-L1-D2-L2-D3

| | | | | | | Amino | Nucleic |
|---|---|---|---|---|---|---|---|
| Compound | | | | | | Acid | Acid |
| Name | D1 | L1 | D2 | L2 | D3 | Sequence | Sequence |
| Compound | Cyclo(RGD)₄ | G₄A | ME194 | (G₄A)₃ | hFH1-5 | SEQ ID | SEQ ID |
| B | (SEQ ID | | VHH | | (SEQ ID | NO: 2 | NO: 126 |
| | NO: 21) | | (SEQ ID | | NO: 16) | | |
| | | | NO: 23) | | | | |
| Compound | Cyclo(RGD)₄ | G₄A | ME194 | (G₄A)₃ | hFH1-5 | SEQ ID | SEQ ID |
| C | (SEQ ID | | VHH | | (SEQ ID | NO: 3 | NO: 127 |
| | NO: 21) | | (SEQ ID | | NO: 16) | | |
| | | | NO: 23) | | | | |

TABLE 3
Fusion Proteins with the sequence, from
N-terminus to C-terminus, of D1-L1-D2

| Com- | | | | Amino | Nucleic |
|---|---|---|---|---|---|
| pound | | | | Acid | Acid |
| Name | D1 | L1 | D2 | Sequence | Sequence |
| Com- | hFH1-5 | (G₄A)₃ | FHRP5₇₋₈ | SEQ ID | SEQ ID |
| pound F | (SEQ ID | | (SEQ ID | NO: 6 | NO: 130 |
| | NO: 16) | | NO: 22) | | |
| Com- | hFH1-5 | (G₄A)₂G₃A | FHRP5₇₋₈ | SEQ ID | SEQ ID |
| pound J | (SEQ ID | G₄S | (SEQ ID | NO: 10 | NO: 134 |
| | NO: 17) | | NO: 22) | | |

TABLE 4
Fusion Proteins with the sequence, from
N-terminus to C-terminus, of D1-D2

| Compound | | | Amino Acid | Nucleic Acid |
|---|---|---|---|---|
| Name | D1 | D2 | Sequence | Sequence |
| Compound A | ME194 VHH | hFH1-5 | SEQ ID NO: | SEQ ID NO: |
| | (SEQ ID | (SEQ ID | 1 | 125 |
| | NO: 23) | NO: 16) | | |
| Compound G | VHH (SEQ | hFH1-5 | SEQ ID NO: | SEQ ID NO: |
| | ID NO: 19) | (SEQ ID | 7 | 131 |
| | | NO: 16) | | |

TABLE 5
Fusion Proteins with the sequence, from
N-terminus to C-terminus, of D2-D1

| Compound | | | Amino Acid |
|---|---|---|---|
| Name | D1 | D2 | Sequence |
| Compound K | hFH1-5 | VHH (SEQ | SEQ ID NO: 11 |
| | (SEQ ID | ID NO: 19) | |
| | NO: 16) | | |
| Compound L | hFH1-5 | VHH (SEQ | SEQ ID NO: 12 |
| | (SEQ ID | ID NO: 19) | |
| | NO: 17) | | |

### Production of Fusion Proteins

Described herein are methods for producing a fusion protein using nucleic acid molecules encoding the fusion proteins, such as the fusion proteins shown in Tables 1-5. The nucleic acid molecule can be operably linked to a suitable control sequence to form an expression unit encoding the protein. The expression unit can be used to transform a suitable host cell, and the transformed host cell can be cultured under conditions that allow the production of the recombinant protein. Optionally, the recombinant protein can be isolated from the medium or from the cells; recovery and purification of the protein may not be necessary in some instances where some impurities may be tolerated. Additional residues may be included at the N- or C-terminus of the protein coding sequence to facilitate purification (e.g., a histidine tag) and, if desired, subsequently removed to form the final protein product.

The fusion protein can be expressed from a single polynucleotide that encodes the entire fusion protein or as multiple (e.g., two or more) polynucleotides that may be expressed by suitable expression systems or may be co-expressed. Polypeptides encoded by polynucleotides that are co-expressed may associate through, e.g., disulfide bonds or other means to form a functional fusion protein. For example, the light chain portion of monoclonal antibody may be encoded by a separate polynucleotide from the heavy chain portion of a monoclonal antibody. When co-expressed in a host cell, the heavy chain polypeptides will associate with the light chain polypeptides to form the monoclonal antibody.

Typically, a nucleic acid encoding the desired fusion protein is generated using molecular cloning methods and is generally placed within a vector, such as a plasmid or virus. The vector is used to transform the nucleic acid into a host cell appropriate for the expression of the fusion polypeptide. Representative methods are disclosed, for example, in Maniatis et al. (Cold Springs Harbor Laboratory, 1989). Many cell types can be used as appropriate host cells, although mammalian cells are often selected because they are able to confer appropriate post-translational modifications. Host cells can include, e.g., a Human Embryonic Kidney (HEK) (e.g., HEK 293) cell, Chinese Hamster Ovary (CHO) cell, L cell, C127 cell, 3T3 cell, BHK cell, COS-7 cell, or any other suitable host cell known in the art.

In one embodiment, a nucleic acid or polynucleotide encoding the fusion protein is provided. In one embodiment, a vector including a nucleic acid or polynucleotide encoding the fusion protein is provided. In one embodiment, a host cell including one or more polynucleotides encoding the fusion protein is provided. In certain embodiments a host cell including one or more fusion expression vectors is provided. The fusion proteins can be produced by expression of a nucleotide sequence in any suitable expression system known in the art. Any expression system may be used, including yeast, bacterial, animal, plant, eukaryotic, and prokaryotic systems. In some embodiments, yeast systems that have been modified to reduce native yeast glycosylation, hyper-glycosylation or proteolytic activity may be used. Furthermore, any in vivo expression systems designed for high level expression of recombinant proteins within organisms known in the art can be used for producing the fusion proteins specified herein. In some embodiments, the factor H fusion protein, as described herein, is produced by culturing one or more host cells including one or more nucleic acid molecules capable of expressing the fusion protein under conditions suitable for expression of the fusion protein. In some embodiments, the factor H fusion protein is obtained from the cell culture or culture medium.

The fusion protein can also be produced using chemical methods to synthesize the desired amino acid sequence, in whole or in part. For example, polypeptides can be synthesized by solid phase techniques, cleaved from the resin, and purified by preparative high-performance liquid chromatography (e.g., Creighton (1983) Proteins: Structures and Molecular Principles, WH Freeman and Co, New York N.Y.). The composition of the synthetic polypeptides can be confirmed by amino acid analysis or sequencing. Additionally, the amino acid sequence of a fusion protein or any part thereof, can be altered during direct synthesis and/or combined using chemical methods with a sequence from other subunits, or any part thereof, to produce a variant polypeptide.

### Isolation/Purification of Fusion Proteins

Secreted, biologically active fusion proteins described herein, such as those described in Tables 1-5, may be purified by techniques, such as high-performance liquid chromatography, ion exchange chromatography, gel electrophoresis, affinity chromatography, e.g., protein A affinity chromatography, size exclusion chromatography, and the like, known in the art. The conditions used to purify a particular protein depend, in part, on factors such as net charge, hydrophobicity, hydrophilicity etc., as would be apparent to a skilled artisan.

### Assays for Fusion Protein Activity

### Hemolytic Assay

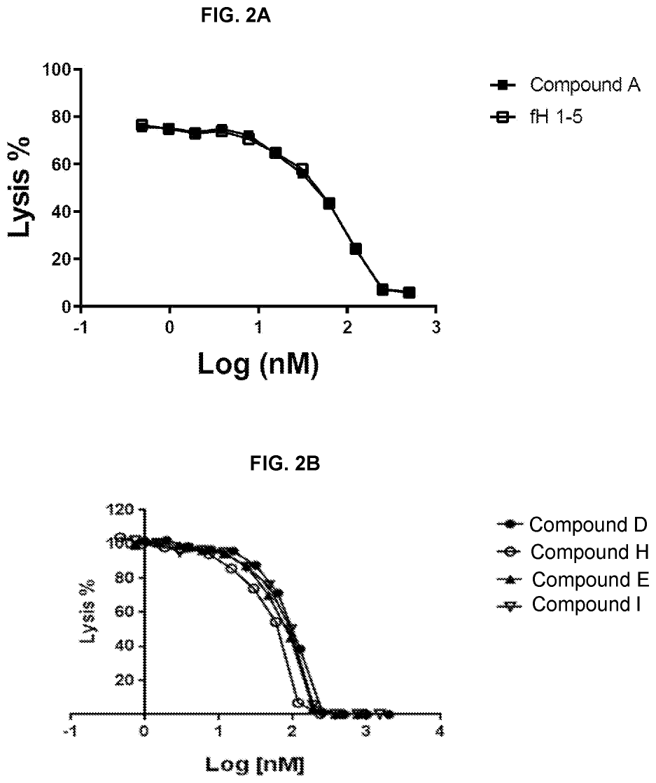

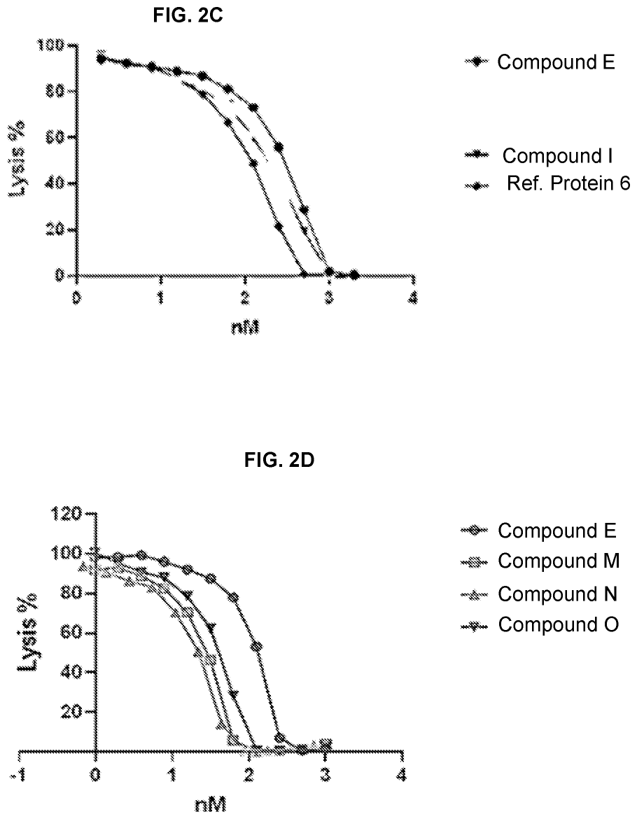

The fusion proteins described herein were assessed for activity using a complement pathway hemolysis assay, which measures complement-mediated lysis of rabbit erythrocytes secondary to activation of the alternative pathway on a cell surface. Rabbit erythrocytes generally activate complement-mediated lysis in mouse or human serum. As serum C3 is activated, C3 convertases, C3 activation fragments, and C5 convertases are deposited on rabbit RBCs. Serum complement alternative pathway activity in the presence of a fusion protein comprising a fragment of factor H and a VHH domain, a fragment of factor H and a fragment of FHRP5 or a fragment of factor H, a VHH, and an integrin binding domain (e.g., the fusion proteins of Tables 1-5), for example, were evaluated in a concentration-dependent manner in human or mouse serum supplemented with Mg++ and EGTA as Ca sequestrant, thus favoring the alternative pathway of complement activation. Incubation of rabbit erythrocytes in normal mouse or human serum causes cell lysis, while addition of nanomolar quantities of a fusion protein comprising a fragment of factor H and a VHH domain, or a fragment of factor H and a fragment of FHRP5, or a fragment of factor H, a VHH domain, and an integrin binding domain, for example, decreases the degree of lysis (see FIGS. 2A-2D). The fusion proteins of the disclosure may exhibit a half maximal inhibitory concentration (IC₅₀) of between about 15 nM to about 250 nM (e.g., between about 15 nM to about 240 nM, between about 15 nM to about 220 nM, between about 200 nM to about 150 nM, between about 15 nM to about 100 nM, between about 15 nM to about 40 nM, or between about 15 nM to about 50 nM. In some embodiments, the fusion protein may exhibit an IC₅₀ of between 19 nM and 240 nM (e.g., between about 19 nM and about 230 nM, about 50 nM and about 240 nM, about 100 nM and about 240 nM, about 150 nM and about 240 nM, about 200 nM and about 240 nM, about 19 nM and 50 nM, about 19 nM and about 100 nM, about 19 nM and about 150 nM, about 19 nM and about 200 nM, and about 19 nM and about 230 nM).

TABLE 6
Complement alternative pathway (CAP) hemolytic assay
half maximal inhibitory concentration (IC₅₀) summary

| Compound | IC₅₀ (nM) |
|---|---|
| A | 48.27 |
| B | 62.63 |
| D | 117.64 |
| E | 165.30 |
| G | 116.40 |
| H | 56.41 |
| I | 137.01 |
| K | 237.10 |
| L | 228.80 |
| M | 28.93 |
| N | 19.61 |
| O | 40.61 |

### Complement Activity Assay

The fusion proteins described herein (e.g., the fusion proteins of Tables 1-5) can be evaluated for complement alternative pathway activity in the fluid phase using a complement alternative pathway assay kit, for example, Complement system Alternative Pathway WIESLAB®, Lund, Sweden. This method combines principles of the hemolytic assay for complement activation with the use of labeled antibodies specific for a neoantigen produced as a result of complement activation. The amount of neoantigen generated is proportional to the functional activity of the alternative pathway. In the Complement system Alternative Pathway kit, wells of the plate are coated with specific activators of the alternative pathway. Serum is diluted in diluent containing specific blockers to ensure that only the alternative pathway is activated. Anti-properdin VHH, for example, can be spiked into the patient's blood in a concentration-dependent manner. During the incubation of the diluted patient serum in the wells, complement is activated by the specific coating. The wells are then washed and C5b-9 is detected with a specific alkaline phosphatase-labelled antibody to the neoantigen as a result of complement activation. The amount of complement activation correlates with the color intensity and is measured in terms of absorbance (optical density (OD)) at 405 nm. The addition of nanomolar quantities of a factor H fusion protein according to the disclosure, for example, decreases the degree of activity. Additional exemplary assays for determining complement pathway activity include those described in Hebell et al., (Science (1991) 254(5028):102-105).

### Pharmaceutical Compositions, Dosage, and Administration

The fusion proteins described herein (see, e.g., Tables 1-5, such as those described in Table 1) can be incorporated into pharmaceutical compositions suitable for administration to a subject. Pharmaceutical compositions including factor H fusion proteins described herein can be formulated for administration at individual doses ranging, e.g., from 0.01 mg/kg to 500 mg/kg. The pharmaceutical composition may contain, e.g., from 0.1 μg/0.5 mL to 1 g/5 mL of the fusion protein. In some embodiments, the pharmaceutical composition described herein contains about 1-200 mg/mL, such as about 30-100 mg/mL, for example about 50 mg/ml (e.g., 50 mg/mL) of the fusion protein.

Compositions including factor H fusion proteins can also be formulated for either a single or multiple dosage regimens. Doses can be formulated for administration, e.g., hourly, bihourly, daily, bidaily, twice a week, three times a week, four times a week, five times a week, six times a week, weekly, biweekly, monthly, bimonthly, or yearly. Alternatively, doses can be formulated for administration, e.g., twice, three times, four times, five times, six times, seven times, eight times, nine times, ten times, eleven times, or twelve times per day.

The pharmaceutical compositions including factor H fusion proteins can be formulated according to standard methods. Pharmaceutical formulation is a well-established art, and is further described in, e.g., Gennaro (2000) Remington: The Science and Practice of Pharmacy, 20th Edition, Lippincott, Williams & Wilkins (ISBN: 0683306472); Ansel et al. (1999) Pharmaceutical Dosage Forms and Drug Delivery Systems, 7th Edition, Lippincott Williams & Wilkins Publishers (ISBN: 0683305727); and Kibbe (2000) Handbook of Pharmaceutical Excipients, American Pharmaceutical Association, 3rd Edition (ISBN: 091733096X).

The pharmaceutical composition can include the fusion protein and at least one pharmaceutically acceptable carrier. As used herein. “pharmaceutically acceptable carrier” includes any and all solvents, dispersion media, coatings, antibacterial and antifungal agents, isotonic and absorption delaying agents, and the like that are physiologically compatible. The term “pharmaceutically acceptable carrier” excludes tissue culture medium including bovine or horse serum. Pharmaceutically acceptable carriers or adjuvants, by themselves, do not induce the production of antibodies harmful to the individual receiving the composition nor do they elicit protection. Therefore, pharmaceutically acceptable carriers are inherently non-toxic and nontherapeutic, and are known to the person skilled in the art. Examples of pharmaceutically acceptable carriers include one or more of water, saline, phosphate buffered saline, dextrose, glycerol, ethanol, and the like, as well as combinations thereof. Some embodiments will include isotonic agents, for example, sugars, polyalcohols such as mannitol, sorbitol, or sodium chloride in the composition. Pharmaceutically acceptable substances include minor amounts of auxiliary substances such as wetting or emulsifying agents, preservatives, stabilizers, or buffers, which enhance the shelf life or effectiveness of the antibody.

The compositions described herein may be prepared in a variety of forms. These include, for example, liquid, semi-solid, and solid dosage forms, such as liquid solutions (e.g., injectable and infusible solutions), dispersions or suspensions, tablets, pills, powders, liposomes, and suppositories. Such formulations can be prepared by methods known in the art such as, e.g., the methods described in Epstein et al. (1985) Proc Natl Acad Sci USA 82:3688; Hwang et al. (1980) Proc Natl Acad Sci USA 77:4030; and U.S. Pat. Nos. 4,485,045 and 4,544,545. Liposomes with enhanced circulation time are disclosed in, e.g., U.S. Pat. No. 5,013,556.

Pharmaceutical compositions including factor H fusion proteins can also be formulated with a carrier that will protect the composition (e.g., a factor H fusion protein) against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known in the art. See, e.g., J. R. Robinson (1978) Sustained and Controlled Release Drug Delivery Systems, Marcel Dekker, Inc., New York.

The final form depends on the intended mode of administration and therapeutic application. Typical compositions are in the form of injectable or infusible solutions, such as compositions similar to those used for passive immunization of humans with other antibodies. The composition(s) can be delivered by, for example, parenteral injection (e.g., intravenous, subcutaneous, intraperitoneal, or intramuscular injection) or by local administration (e.g., directly to the kidneys).

The pharmaceutical compositions can be provided in a sterile form and stable under the conditions of manufacture and storage. The composition can be formulated as a solution, microemulsion, dispersion, liposome, or other ordered structure suitable to high drug concentration. Sterile injectable solutions can be prepared by incorporating the fusion protein in the required amount in an appropriate solvent with one or a combination of ingredients enumerated above, as required, followed by filter sterilization. Generally, dispersions are prepared by incorporating the fusion protein into a sterile vehicle that contains a basic dispersion medium and the required other ingredients from those enumerated above. In the case of sterile powders for the preparation of sterile injectable solutions, exemplary methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin. The form chosen depends, in part, on the intended mode of administration and therapeutic application. For example, compositions intended for systemic or local delivery can be in the form of injectable or infusible solutions. The composition can be formulated, for example, as a buffered solution at a suitable concentration and suitable for storage at 2-8° C. (e.g., 4° C.). A composition can also be formulated for storage at a temperature below 0° ° C. (e.g., −20° C. or)−80° ° C. A composition can further be formulated for storage for up to 2 years (e.g., one month, two months, three months, four months, five months, six months, seven months, eight months, nine months, 10 months, 11 months, 1 year, 1½ years, or 2 years) at 2-8° C. (e.g., 4° C.). Thus, the compositions described herein can be stable in storage for at least 1 year at 2-8° C. (e.g., 4° C.).

The fusion proteins described herein can be administered by a variety of methods known in the art, although for many therapeutic applications, the chosen route/mode of administration is intravenous injection or infusion. The fusion proteins can also be administered by intramuscular or subcutaneous injection. As will be appreciated by the skilled artisan, the route and/or mode of administration will vary depending upon the desired results.

In certain embodiments, the fusion protein may be prepared with a carrier that will protect against rapid release, such as a controlled release formulation, including implants, transdermal patches, and microencapsulated delivery systems.

Biodegradable, biocompatible polymers can be used, such as ethylene vinyl acetate, polyanhydrides, polyglycolic acid, collagen, polyorthoesters, and polylactic acid. Prolonged absorption of injectable compositions can be attained by including in the composition an agent that delays absorption, for example, monostearate salts and gelatin. Many methods for the preparation of such formulations are known to those skilled in the art (e.g., Sustained and Controlled Release Drug Delivery Systems. J. R. Robinson, ed., Marcel Dekker, Inc., New York, 1978). Additional methods applicable to the controlled or extended release of fusion proteins disclosed herein are described, for example, in WO 2016/081884, the entire contents of which are incorporated herein by reference.

The pharmaceutical composition(s) may have a pH of about 5.6-10.0, about 6.0-8.8, or about 6.5-8.0. For example, the pH may be about 6.2, 6.5, 6.75, 7.0, or 7.5, such as pH 7.0. The pharmaceutical compositions may be formulated for oral, sublingual, intranasal, intraocular, rectal, transdermal, mucosal, topical, intravitreal, or parenteral administration. Parenteral administration may include intradermal, subcutaneous (SC, s.q., sub-Q, Hypo), intramuscular (i.m.), intravenous (IV), intraperitoneal (i.p.), intra-arterial, intramedulary, intracardiac, intravitreal (eye), intra-articular (joint), intrasynovial (joint fluid area), intracranial, intraspinal, and intrathecal (spinal fluids) injection or infusion. SC administration may include an SC infusion or an SC push. Any device suitable for parenteral injection or infusion of drug formulations may be used for such administration. For example, the pharmaceutical composition may be contained in a sterile pre-filled syringe.

Additional active compounds can also be incorporated into the composition. In certain embodiments, a fusion protein is co-formulated with and/or co-administered with one or more additional therapeutic agents. When compositions are to be used in combination with a second active agent, the compositions can be co-formulated with the second agent, or the compositions can be formulated separately from the second agent formulation. For example, the respective pharmaceutical compositions can be mixed, e.g., just prior to administration, and administered together or can be administered separately, e.g., at the same or different times. In some embodiments, a fusion protein can be co-formulated and/or co-administered with one or more additional antibodies that bind other targets (e.g., antibodies that bind regulators of the complement alternative pathway). Such combination therapies may utilize lower dosages of the administered therapeutic agents, thus avoiding possible toxicities or complications associated with the various monotherapies. Additionally, the compositions described herein can be co-formulated or co-administered with other therapeutic agents to ameliorate side effects of administering the compositions described herein (e.g., therapeutic agents that minimize risk of infection in an immunocompromised environment, for example, anti-bacterial agents, anti-fungal agents and anti-viral agents).

Preparations of compositions containing factor H fusion proteins can be provided to a subject in combination with pharmaceutically acceptable sterile aqueous or non-aqueous solvents, suspensions, or emulsions. Examples of non-aqueous solvents are propylene glycol, polyethylene glycol, vegetable oil, fish oil, and injectable organic esters. Aqueous carriers include water, water-alcohol solutions, emulsions, or suspensions, including saline and buffered medical parenteral vehicles including sodium chloride solution, Ringer's dextrose solution, dextrose plus sodium chloride solution, Ringer's solution containing lactose, or fixed oils.

Intravenous vehicles can include fluid and nutrient replenishers, electrolyte replenishers, such as those based upon Ringer's dextrose, and the like. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, can be present in such vehicles. A thorough discussion of pharmaceutically acceptable carriers is available in Remington's Pharmaceutical Sciences (Mack Pub. Co., N.J. 1991).

The pharmaceutical compositions can include a “therapeutically effective amount” or a “prophylactically effective amount” of a fusion protein. A “therapeutically effective amount” refers to an amount effective, at dosages, and for periods of time sufficient, to achieve the desired therapeutic result. A therapeutically effective amount of the fusion protein can vary according to factors such as the disease state, age, sex, and weight of the individual, and the ability of the fusion protein to elicit a desired response in the individual. A “prophylactically effective amount” refers to an amount effective, at dosages, and for periods of time sufficient, to achieve the desired prophylactic result. In some embodiments, a prophylactic dose is used in subjects prior to or at an earlier stage of disease where the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time, or the dose may be proportionally reduced or increased as indicated by the exigencies of the therapeutic situation. It is advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. It is to be noted that dosage values can vary with the type and severity of the condition to be alleviated. It is to be further understood that for any particular subject, specific dosage regimens should be adjusted over time according to the individual need and the professional judgment of the administering clinician.

The efficacy of treatment with a fusion protein as described herein can be assessed based on an improvement in one or more symptoms or indicators of the disease state or disorder being treated (e.g., an improvement in one or more symptoms of a complement alternative pathway (CAP)-mediated disease or disorder, such as a kidney disease or disorder mediated by dysregulation of the CAP). An improvement of at least 10% (increase or decrease, depending upon the indicator being measured) in one or more clinical indicators is considered “effective treatment,” although greater improvements are possible, such as 20%. 30%, 40%, 50%, 75%, 90%, or even 100%, or, depending upon the indicator being measured, more than 100% (e.g., two-fold, three-fold, ten-fold, etc., up to and including attainment of a disease-free state.

### Methods of Treatment Using the Fusion Proteins

The complement factor H fusion proteins described herein (see, e.g., Tables 1-5) can be used to treat diseases mediated by complement alternative pathway activation or dysregulation by inhibiting the complement alternative pathway activation in a mammal (e.g., a human). The fusion protein(s) described herein can be used to treat a variety of diseases or disorders mediated by complement alternative pathway activation or dysregulation. Such disorders include, without limitation, kidney disorders, FSGS, IgA nephropathy, MCD, diabetic nephropathy, Alport syndrome, lupus nephritis, membranous nephropathy, acute kidney injury, Goodpasture syndrome, nephrotic syndrome, chronic proteinuria, chronic kidney disease, C3G, dense deposit disease, glomerulonephritis, membranoproliferative glomerulonephritis, polycystic kidney disease, hypertensive nephropathy, nephrosclerosis, aHUS, ischemia reperfusion injury, or rejection of a transplanted organ, such as a kidney.

A therapeutically effective amount of a complement factor H fusion protein, as disclosed herein (e.g., a fusion protein having any one of SEQ ID NOs: 1-15 or a variant thereof with at least 85% (e.g., at least 87%, 88%, 89%, 90%, 91%, 92%, 93%, 94%, 95%, 96%, 97%, 98%, or 99%) sequence identity thereto) can be administered to a mammalian subject in need of such treatment. In some embodiments, the subject is a human patient. The amount administered should be sufficient to inhibit complement activation and/or restore normal complement alternative pathway regulation. The determination of a therapeutically effective dose is within the capability of practitioners in this art; however, as an example, in embodiments of the method described herein utilizing systemic administration of a fusion protein for the treatment diseases mediated by complement alternative pathway activation or dysregulation, an effective human dose will be in the range of 0.01 mg/kg-150 mg/kg ((e.g., from 0.05 mg/kg to 500 mg/kg, from 0.1 mg/kg to 20 mg/kg, from 5 mg/kg to 500 mg/kg, from 0.1 mg/kg to 100 mg/kg, from 10 mg/kg to 100 mg/kg, from 0.1 mg/kg to 50 mg/kg, from 0.5 mg/kg to 25 mg/kg, from 1.0 mg/kg to 10 mg/kg, from 1.5 mg/kg to 5 mg/kg, or from 2.0 mg/kg to 3.0 mg/kg) or from 1 μg/kg to 1,000 μg/kg (e.g., from 5 μg/kg to 1,000 μg/kg, from 1 μg/kg to 750 μg/kg, from 5 μg/kg to 750 μg/kg, from 10 μg/kg to 750 μg/kg, from 1 μg/kg to 500 μg/kg, from 5 μg/kg to 500 μg/kg, from 10 g/kg to 500 μg/kg, from 1 μg/kg to 100 μg/kg, from 5 g/kg to 100 μg/kg, from 10 μg/kg to 100 μg/kg, from 1 μg/kg to 50 μg/kg, from 5 μg/kg to 50 μg/kg, or from 10 μg/kg to 50 μg/kg). The route of administration may affect the recommended dose. Repeated systemic doses are contemplated to maintain an effective level, e.g., to attenuate or inhibit complement activation in a patient's system, depending on the mode of administration adopted.

The compositions and methods described herein may be useful for treatment of a kidney disorder mediated by dysregulation of the CAP, such as FSGS. FSGS is characterized by obliteration of glomerular capillary tufts with increased matrix deposition and scarring (D'Agati et al., Am J Kidney Dis. 43(2):368-382, 2004). The incidence of FSGS has increased over the past decades and it is one of the leading causes of nephrotic syndrome in adults (Korbet, J Am Soc Nephrol. 23(11):1769-1776, 2012). Spontaneous remission is rare (<5%) and presence of persistent nephrotic syndrome indicates a poor prognosis with 50% of patients progressing to end-stage renal disease (ESRD) 6-8 years after initial diagnosis (Korbet, Nephrol Dial Transplant. 14 Suppl 3:68-73, 1999). Primary FSGS is responsible for 3.3% of all the cases of end-stage renal disease (ESRD) resulting from primary kidney disease in the United States. The complement system has been shown to be activated in patients with primary FSGS and elevated levels of plasma Ba, indicative of activation of the alternative pathway, correlates with disease severity. Patients with low serum C3 had a higher percentage of interstitial injury. Furthermore, renal survival was found to be higher in patients with normal serum C3 as compared to those with low serum C3. Low serum C3 is indicative of complement activation. Therefore, activation of the complement system may play a crucial role in the pathogenesis and outcome of FSGS (Liu et al., Scientific Reports, 7: 4095, 2017). In humans, tubulointerstitial deposition of the complement membrane attack complex (C5b-9) is correlated with interstitial myofibroblast accumulation and proteinuria. Experimentally, in focal segmental glomerulosclerosis, the intratubular formation of C5b-9 was found to promote peritubular myofibroblast accumulation. Myofibroblasts may act as sentinel inflammatory cells and deposit extracellular matrix. These cells may also constrict kidney tubules leading to a tubular glomeruli. By this mechanism, complement activation may contribute to tubulointerstitial injury and fibrosis in FSGS (Rangan et al., Kidney Int. 66:1838-1848, 2004). Factor B and factor D deficient mice have lower proteinuria than WT controls in the adriamycin-induced FSGS model, suggesting that activation of CAP has a pathogenic role (Lenderink et al., Am. J. Physiol. Renal Physio. 293:F555-F564, 2007). The alternative pathway of complement is activated in the glomeruli and tubulointerstitium of mice with adriamycin nephropathy (Turnberg et al., J Immunol. 177(6):4094-4102, 2006). Furthermore, complement factor H deficient mice display higher C3b glomerular deposition and more severe kidney damage than wild-type controls (Morigi et al., Sci Rep. 6:28445, 2016), which confirms a previously unrecognized role of C3a in proteinuric progressive nephropathy. Therefore, an inhibitor of the alternative pathway of complement activation can be used to achieve clinical utility in FSGS.

The methods described herein may be useful for treating renal lesions characterized histologically by predominant C3 accumulation in glomeruli in the absence of significant deposition of immunoglobulin (Nester and Smith, Curr. Opin. Nephrol. Hypertens., 22:231-237, 2013) from aberrant regulation of the alternative pathway of complement, also known as C3G.

The methods described herein may be useful for treating dense deposit disease; dense deposit disease is a rare kidney disease leading to persisting proteinuria, hematuria, and nephritic syndrome. Factor H deficiency and concurrent dysfunction in dense deposit disease has been reported in several cases. For example, mutations in factor H have been found in human patients with dense deposit disease. Symptoms of dense deposit disease include, e.g., one or both of hematuria and proteinuria; acute nephritic syndrome; drusen development and/or visual impairment; acquired partial lipodystrophy and complications thereof; and the presence of serum C3 nephritic factor (C3NeF), an autoantibody directed against C3bBb, the C3 convertase of the complement alternative pathway (Appel et al., J. Am. Soc. Nephrol., 16:1392-1404, 2005). Targeting factor H to complement activation sites has therapeutic effects on an individual with dense deposit disease. In some embodiments, administering a composition including a fusion molecule described herein to an individual is effective in treating dense deposit disease. The route of administration may affect the recommended dose. Repeated systemic doses are contemplated to maintain an effective level, e.g., to attenuate or inhibit complement activation in a patient's system, depending on the mode of administration adopted.

The compositions and methods described herein may be useful for treatment of renal inflammation caused by systemic lupus erythematosus (SLE), such as lupus nephritis. Lupus glomerulonephritis includes diverse and complex morphological lesions depending on the proportion of glomeruli affected, by active or chronic lesions, the degree of interstitial inflammation or fibrosis, as well as vascular lesions (Weening et al., J. Am. Soc. Nephrol., 15:241-250, 2004). Lupus nephritis is a serious complication that occurs in a subpopulation of patients with SLE. SLE is the prototypic autoimmune disease resulting in multi-organ involvement. This anti-self response is characterized by autoantibodies directed against a variety of nuclear and cytoplasmic cellular components. These autoantibodies bind to their respective antigens, forming immune complexes that circulate and eventually deposit in tissues. This immune complex deposition causes chronic inflammation and tissue damage. Complement pathways (including the complement alternative pathway) are implicated in the pathology of SLE, and thus fusion proteins provided herein are thus useful for treating lupus nephritis.

The compositions and methods described herein may be useful for treatment of membranous nephropathy (MN), a glomerular disease and the most common cause of idiopathic nephrotic syndrome in nondiabetic, white adults. If untreated, about one-third of MN patients progress to end stage renal disease over 10 years. The incidence of ESRD due to MN in the United States is about 1.9/million per year. Most cases of primary MN (70%) have circulating pathogenic IgG4 autoantibodies to the podocyte membrane antigen PLA2R. Complement components including C3, C4d, and C5b-9 are also commonly present, but not C1q, indicating that the lectin and potentially the alternative pathways of complement activation are involved. Over time, IgG4 and C5b-9 deposition leads to podocyte injury, urine protein excretion and nephrotic syndrome (Couser, Clin J Am Soc Nephrol 12: 983-997, 2017). Mice lacking factor B, an essential component of the alternative pathway of complement activation, did not exhibit C3 and C5b-9 deposition and did not develop albuminuria in a mouse model of MN (Wentian et al., Front Immunol. 9: 1433, 2018). Therefore, complement inhibitors that reduce the amount of C3 and C5 convertases deposited in glomerular lesions, such as the fusion proteins described herein, may be used to effect treatment for this disease.

In some embodiments, the method involves treating a subject with a disease or disorder mediated by complement alternative pathway activation or dysregulation by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M Compound N, and Compound O (e.g., a fusion protein having the amino acid sequence of any one of SEQ ID NOS: 1-15 or a variant thereof with at least 85% sequence identity (or greater) thereto).

In some embodiments, the method involves treating a subject having a kidney disorder by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOS: 1-15) or a variant thereof.

In some embodiments, the method involves treating a subject having membranous nephropathy by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOS: 1-15) or a variant thereof.

In some embodiments, the method involves treating a subject having FSGS by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOs: 1-15) or a variant thereof.

In some embodiments, the method involves treating a subject having glomerulonephritis by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOs: 1-15) or a variant thereof.

In some embodiments, the method involves treating a subject having membranoproliferative glomerulonephritis by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOs: 1-15) or a variant thereof.

In some embodiments, the method involves treating a subject having complement 3 glomerulopathy (C3G) by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOS: 1-15) or a variant thereof.

In some embodiments, the method involves treating a subject having IgA nephropathy by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOS: 1-15) or a variant thereof.

In some embodiments, the method involves treating a subject having MCD by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOs: 1-15) or a variant thereof.

In some embodiments, the method involves treating a subject having diabetic nephropathy by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof.

In some embodiments, the method involves treating a subject having Alport syndrome by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof.

In some embodiments, the method involves treating a subject having lupus nephritis by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof.

In some embodiments, the method involves treating a subject having acute kidney injury by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof.

In some embodiments, the method involves treating a subject having Goodpasture syndrome by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof.

In some embodiments, the method involves treating a subject having nephrotic syndrome by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof.

In some embodiments, the method involves treating a subject having chronic proteinuria by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof.

In some embodiments, the method involves treating a subject having chronic kidney disease by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof.

In some embodiments, the method involves treating a subject having dense deposit disease by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof.

In some embodiments, the method involves treating a subject having polycystic kidney disease by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof.

In some embodiments, the method involves treating a subject having hypertensive nephropathy by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof.

In some embodiments, the method involves treating a subject having nephrosclerosis by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof.

In some embodiments, the method involves treating a subject having aHUS by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof.

In some embodiments, the method involves treating a subject having ischemia reperfusion injury by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof.

In some embodiments, the method involves treating a subject having rejection of a transplanted organ, such as a kidney by administering to the subject a therapeutically effective amount of a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof.

The disclosure further relates to a composition comprising the fusion proteins, as provided above, for use in treatment of a disease or disorder mediated by CAP activation or dysregulation. In some embodiments, the disease or disorder is selected from the group consisting of kidney disorders, FSGS, IgA nephropathy, MCD, diabetic nephropathy, Alport syndrome, lupus nephritis, membranous nephropathy, acute kidney injury, Goodpasture syndrome, nephrotic syndrome, chronic proteinuria, chronic kidney disease, C3G, dense deposit disease, glomerulonephritis, membranoproliferative glomerulonephritis, polycystic kidney disease, hypertensive nephropathy, nephrosclerosis, aHUS, ischemia reperfusion injury, or rejection of a transplanted organ, such as a kidney.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for use in treatment of kidney disorders.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for use in treatment of FSGS.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for use in treatment of membranous nephropathy.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for use in treatment of IgA nephropathy.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for use in treatment of MCD.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOs: 1-15) or a variant thereof (e.g., a fusion protein having at least 85% sequence identity to any one of SEQ ID NOS: 1-15) for use in treatment of diabetic nephropathy.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOS: 1-15) or a variant thereof (e.g., a fusion protein having at least 85% sequence identity to any one of SEQ ID NOS: 1-15) for use in treatment of Alport syndrome.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOS: 1-15) or a variant thereof (e.g., a fusion protein having at least 85% sequence identity to any one of SEQ ID NOS: 1-15) for use in treatment of lupus nephritis.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOS: 1-15) or a variant thereof (e.g., a fusion protein having at least 85% sequence identity to any one of SEQ ID NOs: 1-15) for use in treatment of acute kidney injury.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOS: 1-15) or a variant thereof (e.g., a fusion protein having at least 85% sequence identity to any one of SEQ ID NOS: 1-15) for use in treatment of Goodpasture syndrome.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOS: 1-15) or a variant thereof (e.g., a fusion protein having at least 85% sequence identity to any one of SEQ ID NOS: 1-15) for use in treatment of nephrotic syndrome.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOS: 1-15) or a variant thereof (e.g., a fusion protein having at least 85% sequence identity to any one of SEQ ID NOs: 1-15) for use in treatment of chronic proteinuria.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOs: 1-15) or a variant thereof (e.g., a fusion protein having at least 85% sequence identity to any one of SEQ ID NOs: 1-15) for use in treatment of chronic kidney disease.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOS: 1-15) or a variant thereof (e.g., a fusion protein having at least 85% sequence identity to any one of SEQ ID NOS: 1-15) for use in treatment of C3G.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOS: 1-15) or a variant thereof (e.g., a fusion protein having at least 85% sequence identity to any one of SEQ ID NOS: 1-15) for use in treatment of dense deposit disease.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOS: 1-15) or a variant thereof (e.g., a fusion protein having at least 85% sequence identity to any one of SEQ ID NOS: 1-15) for use in treatment of glomerulonephritis.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOs: 1-15) or a variant thereof (e.g., a fusion protein having at least 85% sequence identity to any one of SEQ ID NOS: 1-15) for use in treatment of membranoproliferative glomerulonephritis.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOs: 1-15) or a variant thereof (e.g., a fusion protein having at least 85% sequence identity to any one of SEQ ID NOS: 1-15) for use in treatment of polycystic kidney disease.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOS: 1-15) or a variant thereof (e.g., a fusion protein having at least 85% sequence identity to any one of SEQ ID NOS: 1-15) for use in treatment of hypertensive nephropathy.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOs: 1-15) or a variant thereof (e.g., a fusion protein having at least 85% sequence identity to any one of SEQ ID NOS: 1-15) for use in treatment of nephrosclerosis.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOS: 1-15) or a variant thereof (e.g., a fusion protein having at least 85% sequence identity to any one of SEQ ID NOs: 1-15) for use in treatment of aHUS.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOS: 1-15) or a variant thereof (e.g., a fusion protein having at least 85% sequence identity to any one of SEQ ID NOS: 1-15) for use in treatment of ischemia reperfusion injury.

The disclosure further relates to a composition including a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID NOS: 1-15) or a variant thereof (e.g., a fusion protein having at least 85% sequence identity to any one of SEQ ID NOS: 1-15) for use in treatment of rejection of a transplanted organ, such as a kidney.

In some embodiments, the disclosure relates to a pharmaceutical composition for treating a disease or disorder mediated by CAP activation or dysregulation. In some embodiments, the disease is kidney disorders, FSGS, IgA nephropathy, MCD, diabetic nephropathy, Alport syndrome, lupus nephritis, membranous nephropathy, acute kidney injury, Goodpasture syndrome, nephrotic syndrome, chronic proteinuria, chronic kidney disease, C3G, dense deposit disease, glomerulonephritis, membranoproliferative glomerulonephritis, polycystic kidney disease, hypertensive nephropathy, nephrosclerosis, aHUS, ischemia reperfusion injury, or rejection of a transplanted organ, such as a kidney.

In some embodiments, the disclosure relates to a pharmaceutical composition for treating kidney disorders, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating FSGS, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating IgA nephropathy, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating diabetic nephropathy, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating acute kidney injury, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating chronic kidney disease, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating membranous nephropathy, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating rejection of a transplanted organ, such as a kidney, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating MCD, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating Alport syndrome, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating nephrotic syndrome, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating lupus nephritis, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating glomerulonephritis, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating membranoproliferative glomerulonephritis, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating Goodpasture syndrome, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating chronic proteinuria, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating C3G, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating dense deposit disease, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating polycystic kidney disease, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating hypertensive nephropathy, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating nephrosclerosis, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating aHUS, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to a pharmaceutical composition for treating ischemia reperfusion injury, containing a fusion protein selected from the group consisting of Compound A, Compound B, Compound C, Compound D, Compound E, Compound F, Compound G, Compound H, Compound I, Compound J, Compound K, Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) as an active ingredient.

In some embodiments, the disclosure relates to use of a composition comprising a fusion protein, as provided above, for the manufacture of a medicament for treating a disease or disorder mediated by CAP activation or dysregulation. In some embodiments, the disease is selected from the group consisting of kidney disorders, FSGS, IgA nephropathy, MCD, diabetic nephropathy, Alport syndrome, lupus nephritis, membranous nephropathy, acute kidney injury, Goodpasture syndrome, nephrotic syndrome, chronic proteinuria, chronic kidney disease, C3G, dense deposit disease, glomerulonephritis, membranoproliferative glomerulonephritis, polycystic kidney disease, hypertensive nephropathy, nephrosclerosis, aHUS, ischemia reperfusion injury, or rejection of a transplanted organ, such as a kidney.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for kidney disorders.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for FSGS.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for IgA nephropathy.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for diabetic nephropathy.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for acute kidney injury.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for chronic kidney disease.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for membranous nephropathy.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for rejection of a transplanted organ, such as a kidney.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for MCD.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for Alport syndrome.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for nephrotic syndrome.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for chronic proteinuria.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for lupus nephritis.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for membranoproliferative glomerulonephritis.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for glomerulonephritis.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for Goodpasture syndrome.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for C3 glomerulopathy.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for dense deposit disease.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for polycystic kidney disease.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for hypertensive nephropathy.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for nephrosclerosis.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for aHUS.

In some embodiments, the disclosure relates to use of a composition comprising a fusion protein selected from the group consisting of Compound A, Compound B, Compound C, Compound D, Compound E, Compound F, Compound G, Compound H, Compound I, Compound J, Compound K Compound L, Compound M, Compound N, and Compound O (e.g., a fusion protein having the amino acid sequence of any one of SEQ ID Nos: 1-15) or a variant thereof (e.g., a fusion protein having at least 85% sequence identity to any one of SEQ ID Nos: 1-15) for the manufacture of a medicament for ischemia reperfusion injury.

### EXAMPLES

The following examples are put forth so as to provide those of ordinary skill in the art with a disclosure and description of how the methods and compounds claimed herein are performed, made. They are intended to be purely exemplary and are not intended to limit the scope of the disclosure.

### Example 1. In Silico Design and Construction of Kidney-Localizing Factor H Fusion Proteins

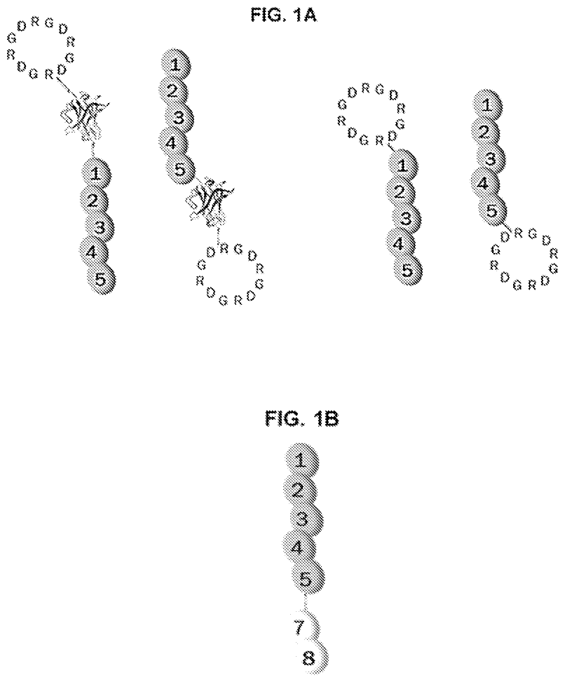

Constructs including various combinations of the Factor H N-terminus functional domain, a VHH domain and a poly-arginylglycylaspartic acid (RGD) domain were designed in silico. Exemplary constructs are illustrated in FIGS. 1A and 1B.

The amino acid sequences of the constructs shown in FIGS. 1A and 1B were provided to GeneArt (ThermoFisher) for codon optimization and gene synthesis. Nucleotide sequences were cloned into a proprietary vector for expression in mammalian cells. Plasmid DNA was then transiently transfected into HEK293 and CHO cells. After 4-5 days, supernatants were harvested. The concentrations of fusion proteins were determined by SDS-PAGE and densitometry.

### Example 2. Inhibition of the Alternative Pathway Using Fusion Proteins

Compounds according to the disclosure were tested for their ability to inhibit the alternative pathway using the CAP-specific hemolytic assay. Results are shown in FIGS. 2A, 2B, 2C, and 2D. Briefly, rabbit red blood cells were washed and added to 10% human serum containing Mg²⁺ and EGTA. Serial dilutions of inhibitors were added, and the cells were incubated for 30 min at 37° C. Cells were removed by centrifugation and the amount of cell lysis was determined by measuring the absorbance of the supernatant at 415 nm. Inhibition of the alternative pathway lysis was shown for all molecules tested, confirming that the molecules were functional as expected.

### Example 3. Visualization of Fusion Proteins in the Kidney

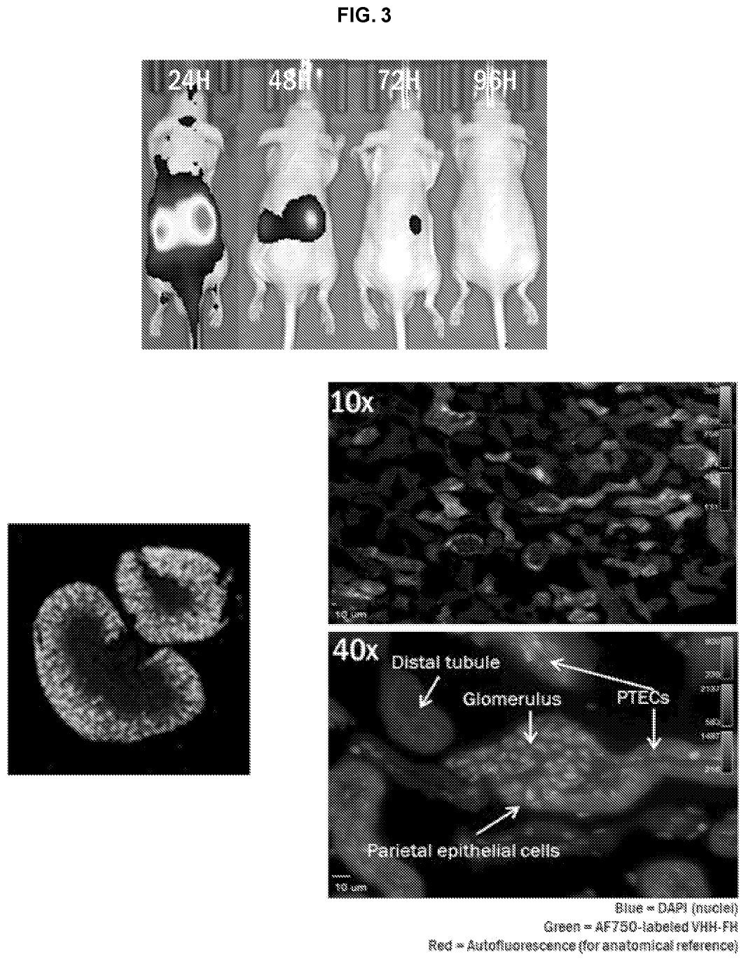

In vivo imaging of fluorescently-labeled constructs was performed in female J:NU outbred nude mice (Jackson Laboratories, Bar Harbor, ME). FIG. 3 shows representative longitudinal in vivo imaging in the same subject acquired over a period of 4 days. For image acquisition, subjects were maintained under 2-3% isoflurane anesthesia on the warmed imaging platform within the IVIS Spectrum Imaging System (PerkinElmer Inc., Waltham, MA). Fluorescent imaging analysis was performed using Living Image 4.5.1 software (PerkinElmer Inc., Waltham, MA), with automatic 2D epi-illumination exposure settings, field of view (FOV) C, F/Stop 2, medium binning and 800 nm emission/750 nm excitation filters. Subjects received 1 mg/kg of AlexaFluor 750-labeled test article via volume-normalized 100 μL intravenous tail vein injection. All animal studies were conducted according to provisions of the Animal Welfare Act and the principles of the Guide for the Care and Use of Laboratory Animals. All procedures were approved by the IACUC at Alexion Pharmaceuticals, Inc., New Haven, CT, protocol #917103.

FIG. 3 also shows the kidney from the same mouse imaged via fluorescent microscopy. The signal shows biodistribution of the tested compound, with selectivity for the apical side of parietal and proximal tubule epithelial cells. Kidney residence time was extended by the presence of the VHH and RGD-containing motifs.

### Example 4. Pharmacokinetic Determination of Select Fusion Proteins

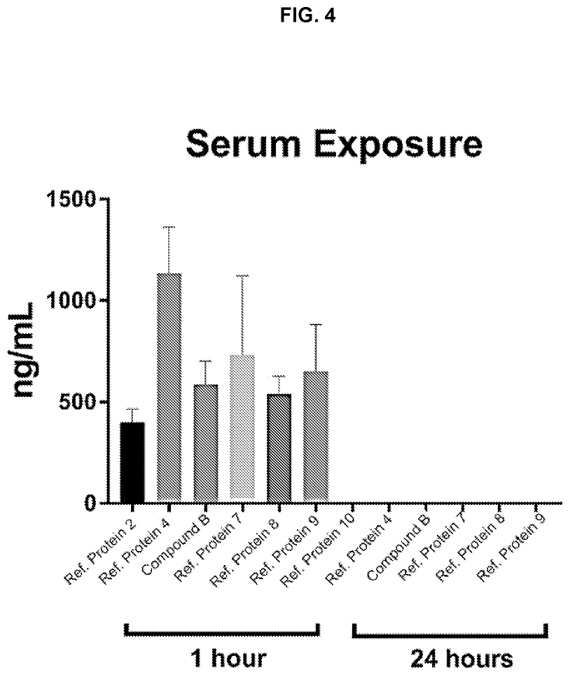

Single-dose pharmacokinetic exposures were determined for select compounds in female J:NU outbred nude mice (Jackson Laboratories, Bar Harbor, ME). Serum exposures were measured by an electrochemiluminescent detection assay developed on the MSD (Meso Scale Discovery, Rockville, Maryland) platform. FIG. 4 shows the PK of compounds at 1- and 24-hours post IV bolus administration. In contrast to the kidney residence kinetics shown in FIG. 3, in serum, the compounds were below the lower limit of detection by 24 hours post-administration, confirming the targeting specificity of the compounds.

### Example 5. Determination of Therapeutic Efficacy in FSGS Model

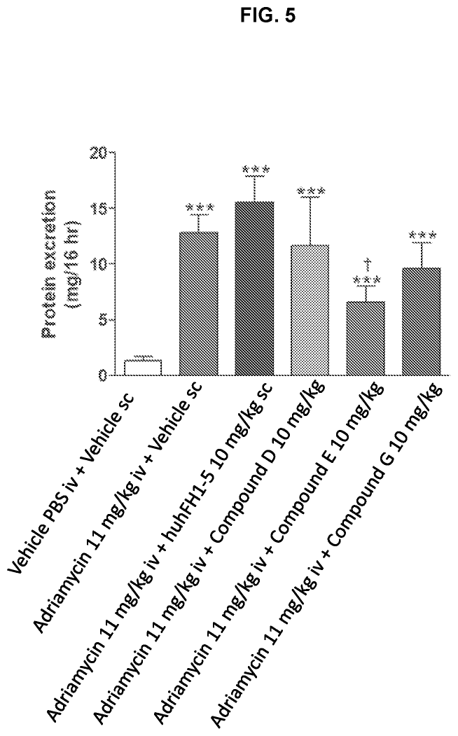

Compound D, Compound E, and Compound G were evaluated for therapeutic efficacy in the Adriamycin-induced nephropathy mouse model of focal segmental glomerulosclerosis (FSGS). FIG. 5 shows therapeutic effects on urine protein. Factor H₁₋₅ alone was not sufficient for providing significant benefit. Efficacy was enhanced by the presence of the VHH and RGD-containing motifs.

### Example 6. Determination of Therapeutic Efficacy in FSGS Model

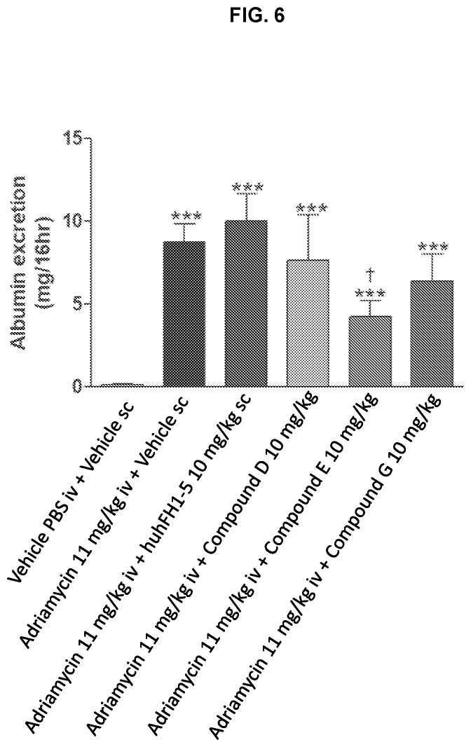

Compound D, Compound E, and Compound G were evaluated for therapeutic efficacy in the Adriamycin-induced nephropathy mouse model of FSGS. FIG. 6 shows therapeutic effects on urine albumin. Factor H₁₋₅ alone was not sufficient for providing significant benefit. Efficacy was enhanced by the presence of the VHH and RGD-containing motifs.

### Example 7. Determination of Therapeutic Efficacy in FSGS Model

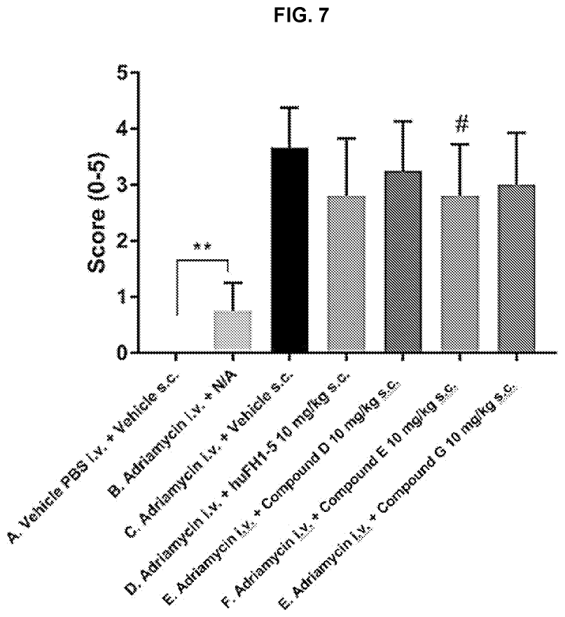

Compound D, Compound E, and Compound G were evaluated for therapeutic efficacy in the Adriamycin-induced nephropathy mouse model of FSGS. FIG. 7 shows therapeutic effects on tubular protein. Factor H₁₋₅ alone was not sufficient for providing significant benefit.

### Example 8. Visualization of Kidneys in FSGS Model after Fusion Protein Administration

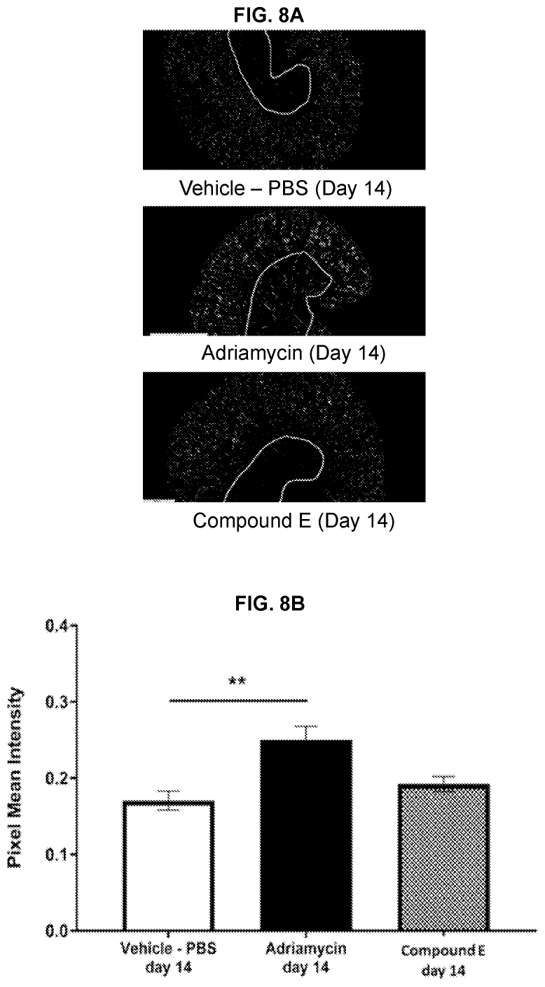

Immunofluorescent C3 activation product staining was performed on kidney sections from mice in the Adriamycin-induced nephropathy mouse model of FSGS 7 days following therapeutic administration of Compound E (study day 14). FIG. 8A exemplifies regions-of-interest (ROIs) manually applied to approximate the kidney medulla for performing area-normalized C3 fragment mean pixel intensity analysis and evaluating local complement alternative pathway (CAP) activation. FIG. 8B qualitatively illustrates the medullary CAP regulation that occurs following treatment with Compound E.

### Example 9. Determination of Therapeutic Efficacy in FSGS Model

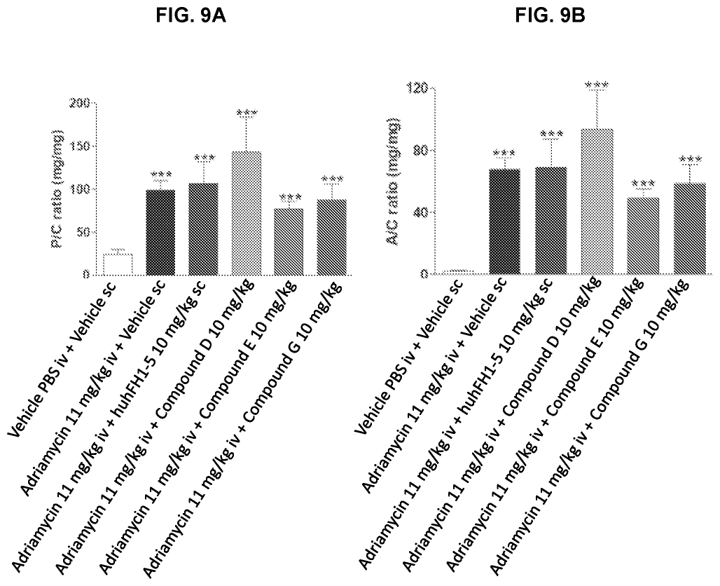

Compound D, Compound E, and Compound G were evaluated for therapeutic efficacy in the Adriamycin-induced nephropathy mouse model of FSGS. On study day 13, the animals were individually placed into metabolism cages for 16 hours. Collected urine was analyzed for albumin, protein, and creatinine by Cobas analyzer. Calculated urine protein/creatinine (FIG. 9A) and albumin/creatinine (FIG. 9B) ratios demonstrate positive but statistically insignificant benefit trends following treatment with Compound E.

### Example 10. Characterization of Fusion Protein Purity

Proteins were evaluated by non-reducing SDS-PAGE gel to confirm purity and molecular weight.

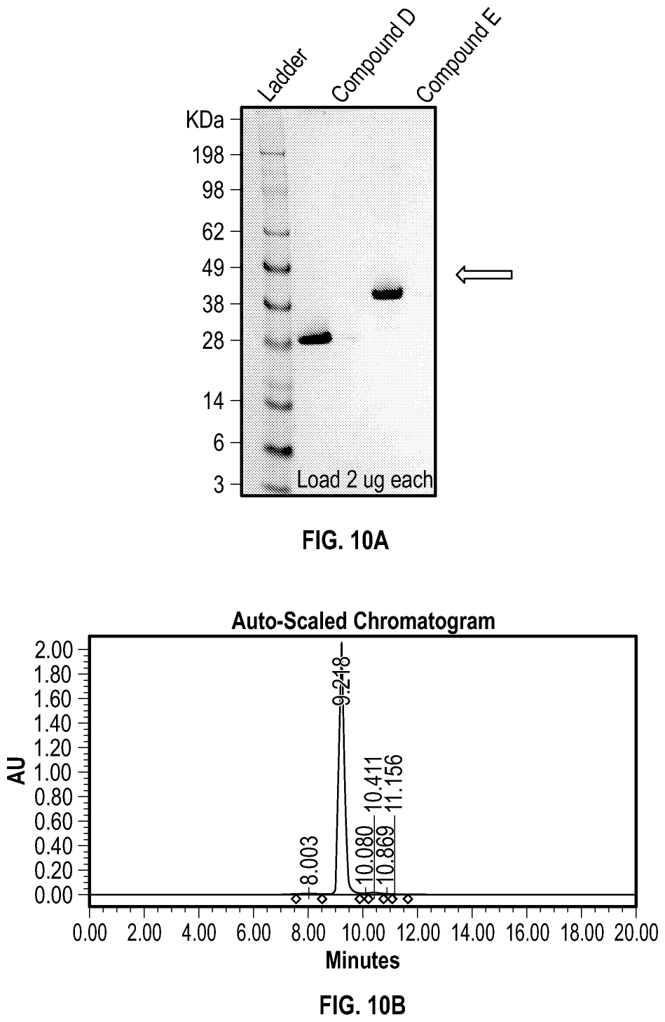

Most fusion proteins were readily purified by anion exchange chromatography, followed by hydrophobic interaction chromatography (HIC) to high levels of purity. Anion exchange was performed on 34 mL Capto Q Impres. HIC (HiTrap Phenyl FF (HS)) was equilibrated in buffer A (20 mM Tris-HCl, 3M NaCl, pH 8.2), and eluted with buffer B (20 mM Tris-HCl, pH 8.2). Purity was greater than 95% for Compound E. Non-reducing SDS-PAGE showed a single band at 2 μg loading per well after two-step purification. Select proteins were readily purified by Protein A chromatography to high levels of purity. The concentrations of purified fusion proteins were determined by UV spectroscopy absorbance at 280 nm corrected for molar extinction coefficient. Purity was assessed by SDS-PAGE and size-exclusion chromatography (SEC) HPLC. Exemplary harvested cell culture supernatants assessed via SDS-PAGE and purified protein via SEC-HPLC are shown in FIGS. 10A and 10B, respectively.

### Example 11. Characterization of Fusion Proteins Using Mass Spectrometry

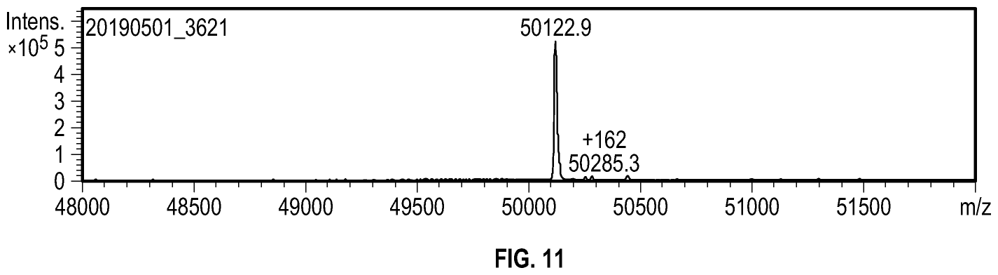

Select compounds were evaluated to determine intact molecular weights by electrospray ionization-time of flight (ESI-TOF) mass spectrometry. FIG. 11 shows confirmation with the expected theoretical molecular weight for representative Compound E.

### Example 12. Characterization of Fusion Proteins Using Dynamic Light Scattering

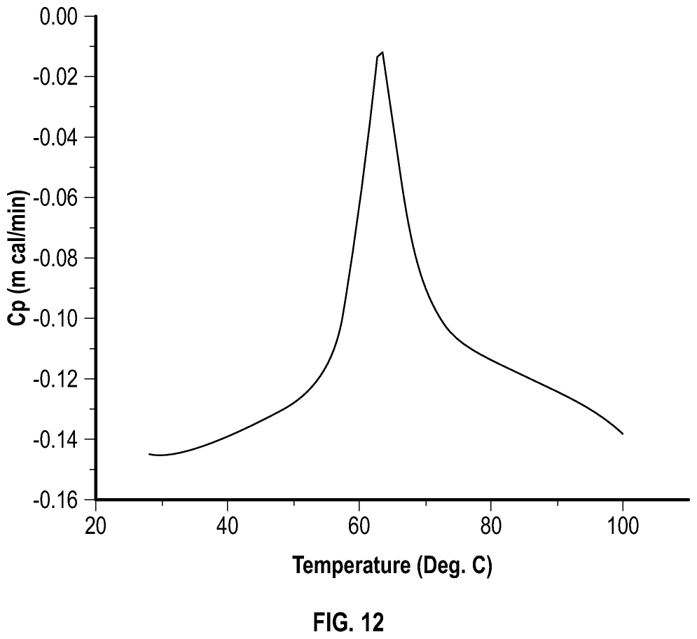

Melting points were determined by dynamic light scattering (DLS). Compounds of interest were first diluted to 1 mg/mL in PBS, PH 7.4 and evaluated with a scan rate of 60° C.-Hour. FIG. 12 is an exemplary scan and shows satisfactory melting point as measured by DLS for Compound E. Other compounds gave similar results.

### Example 13. Characterization of Fusion Proteins Using Size Exclusion Chromatography

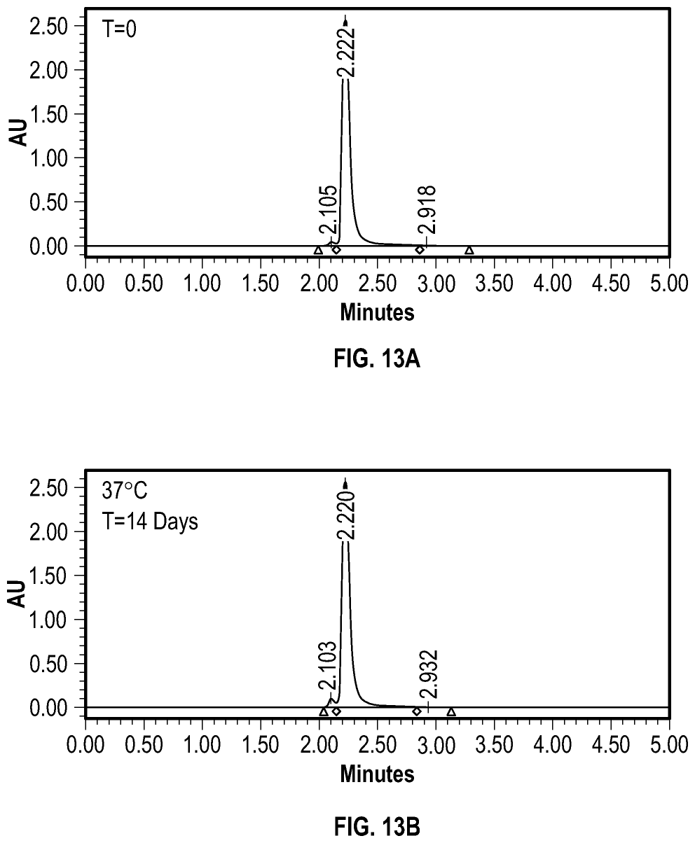

A size exclusion chromatogram of Compound E was obtained following 0 and 14 days of incubation at 37° C. The relative percent of aggregate protein and the relative percent of the intact fusion protein were calculated from the chromatograms measured after 0 days (FIG. 13A), with 1.1% aggregate and 98.4% fusion protein, and 14 days (FIG. 13B), with 2.4% aggregate and 97.4% fusion protein. The negligible 1.3% increase in aggregate protein over the 14-day period indicates the fusion protein is stable over this length of time.

### Example 14. Characterization of Fusion Protein by Hydrophobic Interactions

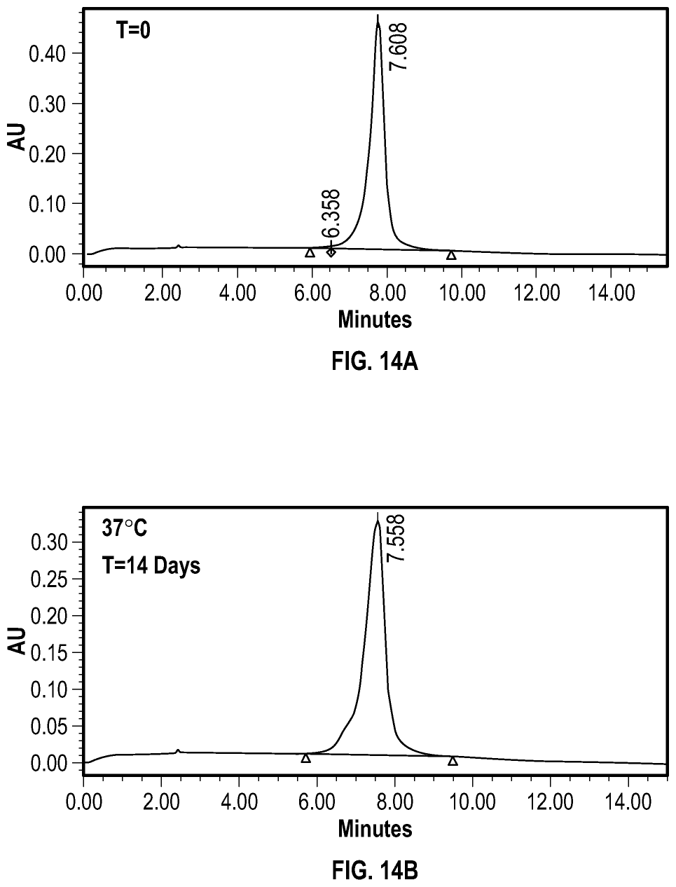

A hydrophobic interaction chromatogram of Compound E was obtained following 0 and 14 days of incubation at 37° C. The resulting chromatograms show the retention time and area under the peak remain unchanged from 0 days to 14 days, as shown in FIGS. 14A and 14B, indicating that the fusion protein is stable over this time period.

### Example 15. Characterization of Fusion Protein Stability Using Capillary Electrophoresis

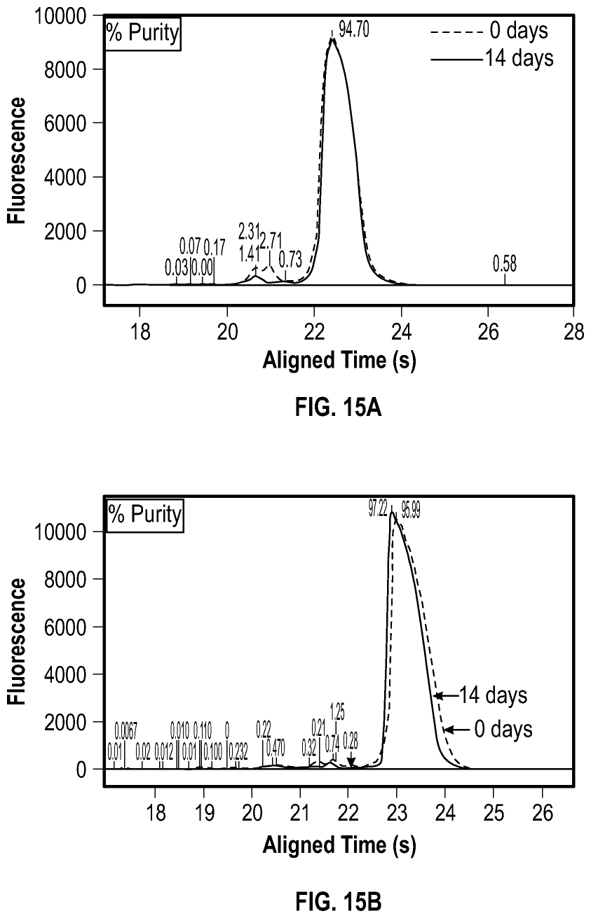

Stability of the compounds over time (14 days at 37° C.) was evaluated via CE-SDS. FIG. 15A shows Compound E non-reduced CE-SDS chromatogram at time zero and after 14 days. FIG. 15B shows the reduced CE-SDS chromatogram after zero days and after 14 days. The profiles at day zero and day 14 were similar, confirming the stability of the compound. No appreciable difference was shown under reducing vs non-reducing conditions, additionally confirming protein stability.

### Example 16. Characterization of Fusion Protein Stability Using Iso-Electric Capillary Electrophoresis

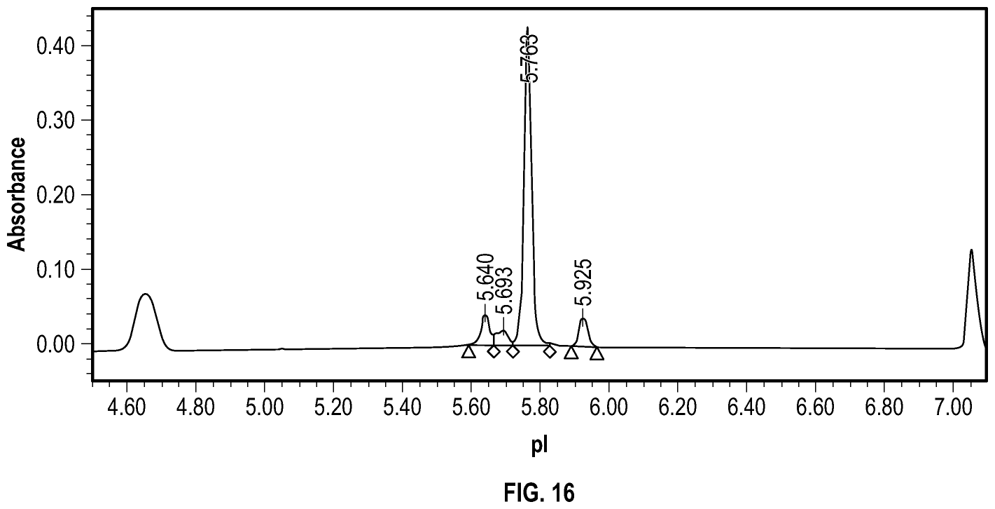

Iso-electric capillary electrophoresis (ICE) was performed to determine stability and lack of charge heterogeneity according to standard techniques. FIG. 16 shows a representative Compound E ICE result. Six total replicates confirmed a consistent stability and manufacturability profiles for Compound E.

### Example 17. Characterization of Fusion Protein Stability Over Using Mass Spectrometry

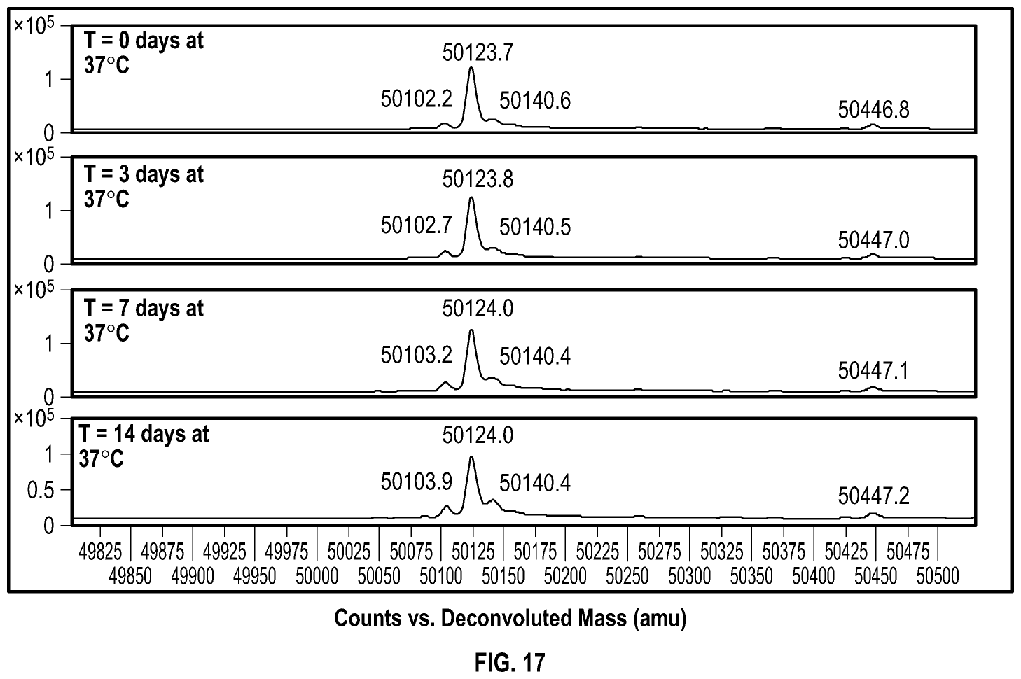

Stability of intact molecular weight of the compounds was measured at various time points over 14 days at 37° C. was evaluated via MS via standard techniques. FIG. 17 shows Compound E at time zero, day 3, day 7, and day 14. The profiles were all similar, confirming the stability of Compound E. Similar results were obtained for the compounds Compound D and Compound G.

### Example 18. Characterization of Binding of Fusion Proteins to C3b

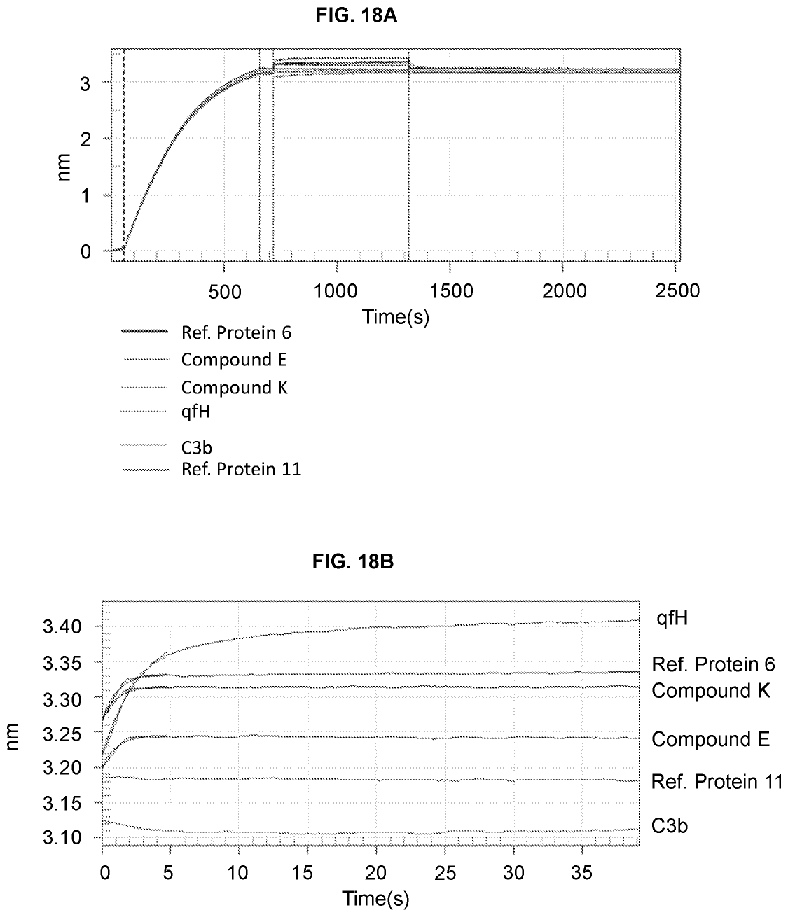

Binding of the compounds to C3b was assessed by bio-layer interferometry (BLI). Biotinylated C3b was immobilized to streptavidin biosensor tips and exposed to analytes of molar-equivalent concentrations diluted in kinetics buffer. FIG. 18A shows expected binding profiles of Compound E and Compound K as compared to non-binding controls (reference protein 11, which is an anti-C5 VHH reference protein used as a negative control, and reference protein 6, which is an anti-HSA factor H-VHH fusion protein used as a positive control) represented by shifts in optical thickness on the sensor tips. FIG. 18B shows a 40-second, blown-up section of the binding curve of FIG. 18A, in which the t=0 seconds timepoint on FIG. 18B corresponds to the t=720 seconds timepoint on FIG. 18A. These results confirm the binding of the compounds to C3b.

### Example 19. Measuring Effect of Fusion Protein on Complement Alternative Pathway Regulation

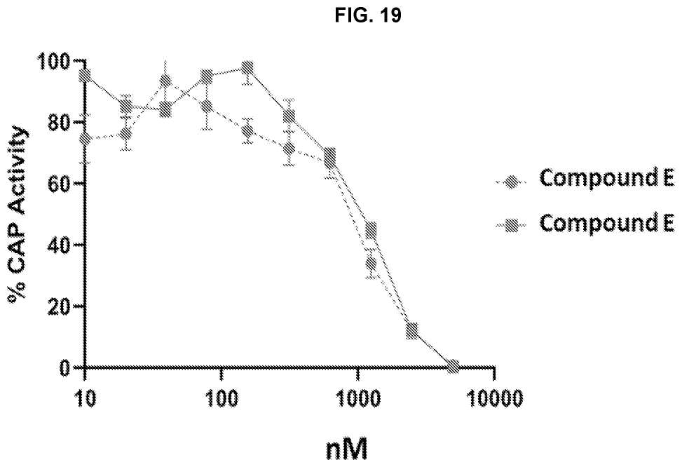

Regulation of the complement alternative pathway (CAP) in normal human serum (NHS) by Compound E was assessed according to manufacturer's instructions in the Complement system Alternative Pathway WIESLAB® assay. A comparable, dose-dependent inhibition of the C5b-9 neoantigen expressed during MAC formation was observed across two tested lots of NHS under otherwise identical conditions, as shown in FIG. 19.

### Example 20. Pharmacokinetic Assessment of Compound E in Wild-Type Mice

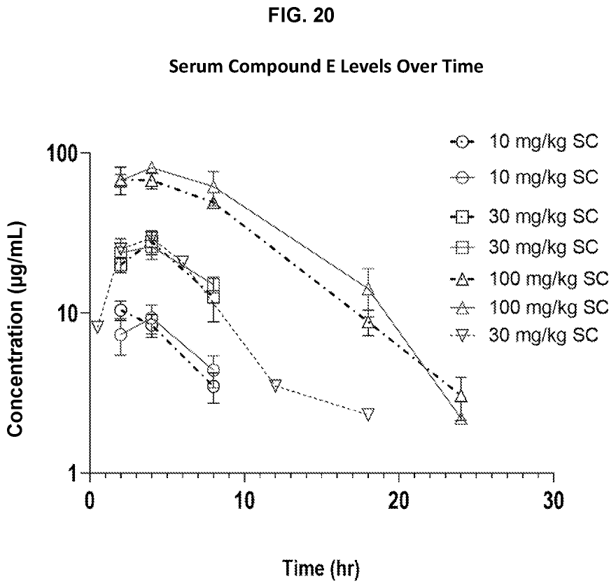

Single-dose pharmacokinetic exposures were determined for Compound E across a range of subcutaneous (SC) doses in wild-type male C57BI/6J mice (Jackson Laboratories, Bar Harbor, ME). FIG. 20 shows composite data from two separate studies illustrating dose-dependent pharmacokinetics of Compound E administered at 10, 30, and 100 mg/kg. Serum exposures were measured by LC-MS/MS.

### Example 21. Pharmacokinetic Assessment of Compound E in Cynomolgus Monkeys

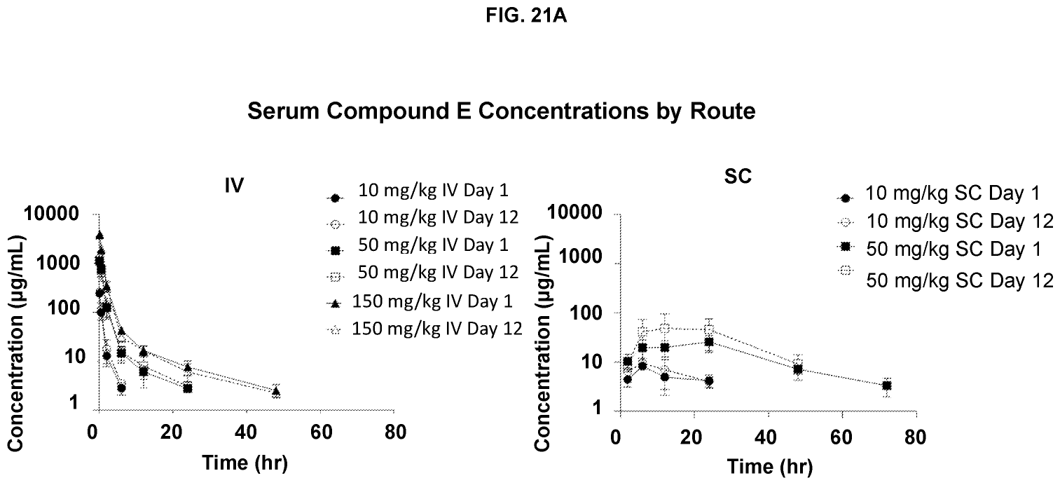

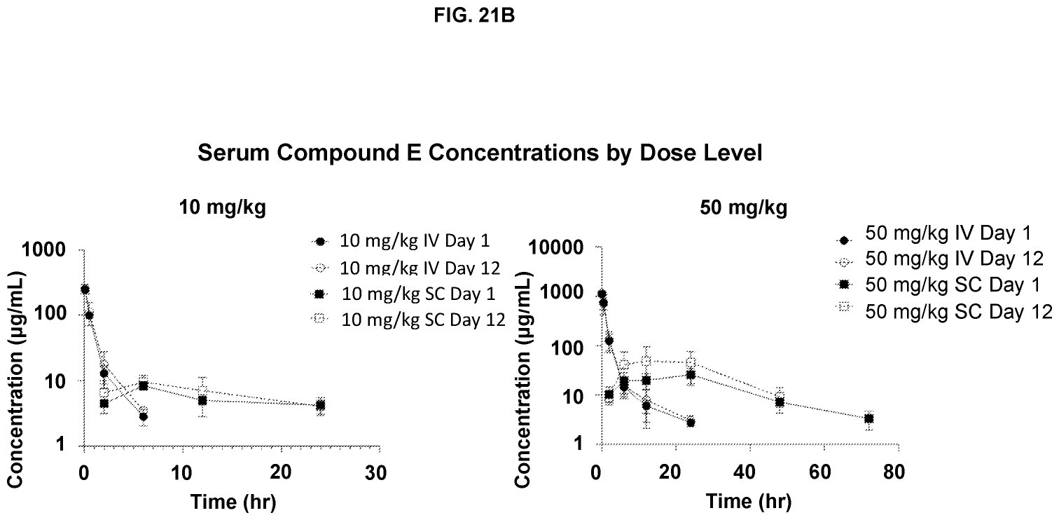

Single-dose pharmacokinetic exposures were determined for Compound E across a range of intravenous (IV) and subcutaneous (SC) doses in female cynomolgus (Macaca fascicularis) monkeys (Charles River Laboratories, Inc., Mattawan, MI). FIG. 21A shows serum pharmacokinetics of Compound E following both an initial dose given on study day 0 and a fourth dose administered on study day 12. FIG. 21B includes the data from FIG. 21A re-plotted to compare equivalent dose levels given by IV or SC route of administration. Serum exposures were measured by LC-MS/MS.

### Other Embodiments

All publications, patents, and patent applications mentioned in this specification are herein incorporated by reference to the same extent as if each independent publication or patent application was specifically and individually indicated to be incorporated by reference. While particular embodiments are herein described, one of skill in the art will appreciate that further modifications and embodiments are encompassed including variations, uses or adaptations generally following the principles described herein and including such departures from the present disclosure that come within known or customary practice within the art and may be applied to the essential features hereinbefore set forth, and follows in the scope of the claims.

1. A fusion protein having the structure, from N-terminus to C-terminus:
D1-L1-D2-L2-D3,
wherein
D1 comprises a fragment of complement factor H (FH);
L1 is absent, is a covalent bond, or is an amino acid sequence of at least one amino acid;
D2 comprises a VHH or is absent;
L2 is absent, is a covalent bond, or is an amino acid sequence of at least one amino acid; and
D3 is an integrin recognition domain.

2. The fusion protein of claim 1, wherein D1 comprises one or more FH short consensus repeat (SCR) domains, optionally wherein the one or more SCR domains are selected from the group consisting of SCR 1, 2, 3, 4, 5, and 6.

3. The fusion protein of claim 2, wherein the FH SCR domains are selected from the group consisting of SCR 1-4; 1-5; and 1-6.

4. The fusion protein of any one of claims 1-3, wherein the VHH of D2 comprises a single-domain antibody.

5. The fusion protein of any one of claims 1-4, wherein the VHH of D2 comprises a camelid single domain antibody.

6. The fusion protein of any one of claims 1-5, wherein the integrin recognition domain of D3 comprises an integrin recognition domain comprising an arginylglycylaspartic acid (RGD) peptide motif.

7. The fusion protein of any one of claims 1-6, wherein the integrin recognition domain of D3 comprises a cyclo(RGD)4 peptide motif.

8. The fusion protein of any one of claims 1-7, wherein L1 and L2 comprise the same amino acid sequence.

9. The fusion protein of any one of claims 1-7, wherein L1 and L2 comprise different amino acid sequences.

10. The fusion protein of claim 8 or 9, wherein L1 and/or L2 are selected from the group consisting of: (G₄A)₂G₃AG₄S, G₄SDAA, (G₄A)₂G₄S, G₄AG₃AG₄S, GGGGAGGGGAGGGGS, GGGGGGGGSGGGGS, G₄S, (G₄S)₂, (G₄S)₃, (G₄S)₄, (G₄S)₅, (G₄S)₆, (EAAAK)₃, PAPAP, G₄SPAPAP, PAPAPG₄S, GSTSGKSSEGKG, (GGGDS)₂, (GGGES)₂, GGGDSGGGGS, GGGASGGGGS, GGGESGGGGS, ASTKGP, ASTKGPSVFPLAP, G₃P, G₇P, PAPNLLGGP, G₆, G₁₂, APELPGGP, SEPQPQPG, (G₃S2)₃, GGGGGGGGGSGGGS, GGGGSGGGGGGGGGS, (GGSSS)₃, (GS₄)₃, G₄A(G₄S)₂, G₄SG₄AG₄S, G₃AS(G₄S)₂, G₄SG₄ASG₄S, G₄SAG₃SG₄S, (G₄S)₂AG₃S, G₄SAG₃SAG₃S, G₄D(G₄S)₂, G₄SG₄DG₄S, (G₄D)₂G₄S, G₄E(G₄S)₂, G₄SG₄EG₄S, (G₄E)₂G₄S, G₄SDA, G₄A, and (G₄A)₃.

11. The fusion protein of claim 10, wherein L1 and/or L2 are selected from the group consisting of: (G₄A)₂G₃AG₄S, G₄SDAA, (G₄A)₂G₄S, G₄SDAA, (G₄S)₄, G₄AG₃AG₄S, G₄A, and (G₄A)₃.

12. The fusion protein of claim 1, wherein
(a) D1 comprises the FH SCR domains 1-5; L1 comprises G₄A; D2 is absent; L2 is absent; and D3 comprises cyclo(RGD)₄;
(b) D1 comprises the FH SCR domains 1-5; L1 is absent; D2 comprises the VHH; L2 comprises G₄A; and D3 comprises cyclo(RGD)₄;
(c) D1 comprises the FH SCR domains 1-5; L1 comprises G₄A; D2 is absent; L2 comprises G₄A; and D3 comprises cyclo(RGD)₄;
(d) D1 comprises the FH SCR domains 1-5; L1 is absent; D2 comprises a VHH; L2 comprises G₄A; and D3 comprises cyclo(RGD)₄;
(f) D1 comprises the FH SCR domains 1-5; L1 is absent; D2 comprises a VHH; L2 comprises G₄A; and D3 comprises cyclo(RGD)₄;
(g) D1 comprises the FH SCR domains 1-6; L1 is absent; D2 comprises a VHH; L2 comprises G₄A; and D3 comprises cyclo(RGD)₄; or
(h) D1 comprises the FH SCR domains 1-5; L1 comprises G₄A; D2 comprises a VHH; L2 comprises G₄A; and D3 comprises cyclo(RGD)₄.

13. The fusion protein of claim 1, wherein the fusion protein
(a) has an amino acid sequence of SEQ ID NO: 4, or a variant thereof having up to 10 amino acid substitutions, additions, or deletions;
(b) has an amino acid sequence of SEQ ID NO: 5, or a variant thereof having up to 10 amino acid substitutions, additions, or deletions;
(c) has an amino acid sequence of SEQ ID NO: 8, or a variant thereof having up to 10 amino acid substitutions, additions, or deletions;
(d) has an amino acid sequence of SEQ ID NO: 9, or a variant thereof having up to 10 amino acid substitutions, additions, or deletions;
(e) has an amino acid sequence of SEQ ID NO: 13, or a variant thereof having up to 10 amino acid substitutions, additions, or deletions; or
(f) has an amino acid sequence of SEQ ID NO: 14, or a variant thereof having up to 10 amino acid substitutions, additions, or deletions;
(g) has an amino acid sequence of SEQ ID NO: 15, or a variant thereof having up to 10 amino acid substitutions, additions, or deletions.

14. The fusion protein of claim 1, wherein the fusion protein
(a) has an amino acid sequence with at least 85% sequence identity to SEQ ID NO: 4;
(b) has an amino acid sequence with at least 85% sequence identity to SEQ ID NO: 5;
(c) has an amino acid sequence with at least 85% sequence identity to SEQ ID NO: 8;
(d) has an amino acid sequence with at least 85% sequence identity to SEQ ID NO: 9;
(e) has an amino acid sequence with at least 85% sequence identity to SEQ ID NO: 13;
(f) has an amino acid sequence with at least 85% sequence identity to SEQ ID NO: 14;
(g) has an amino acid sequence with at least 85% sequence identity to SEQ ID NO: 15.

15. A fusion protein comprising the structure, from N-terminus to C-terminus:
D1-L1-D2, wherein:
D1 comprises a FH fragment, such as FH1-5;
L1 comprises a linker or is absent; and
D2 comprises a factor H-related protein 5 (FHRP5) domain, such as FHRP domains 7 and 8.

16. The fusion protein of claim 15, wherein L1 is selected from the group consisting of: G₄A, (G₄A)₃, (G₄A)₂G₃AG₄S, G₄SDAA, (G₄A)₂G₄S, G₄AG₃AG₄S, GGGGAGGGGAGGGGS, GGGGSGGGGSGGGGS, G₄S, (G₄S)₂, (G₄S)₃, (G₄S)₄, (G₄S)₅, (G₄S)₆, (EAAAK)₃, PAPAP, G₄SPAPAP, PAPAPG₄S, GSTSGKSSEGKG, (GGGDS)₂, (GGGES)₂, GGGDSGGGGS, GGGASGGGGS, GGGESGGGGS, ASTKGP, ASTKGPSVFPLAP, G₃P, G₇P, PAPNLLGGP, G₆, G₁₂, APELPGGP, SEPQPQPG, (G₃S2)₃, GGGGGGGGGGGGS, GGGGSGGGGGGGGGS, (GGSSS)₃, (GS₄)₃, G₄A(G₄S)₂, G₄SG₄AG₄S, G₃AS(G₄S)₂, G₄SG₃ASG₄S, G₄SAG₃SG₄S, (G₄S)₂AG₃S, G₄SAG₃SAG₃S, G₄D(G₄S)₂, G₄SG₄DG₄S, (G₄D)₂G₄S, G₄E(G₄S)₂, G₄SG₄EG₄S, (G₄E)₂G₄S, and G₄SDA.

17. The fusion protein of claim 16, wherein L1 is selected from the group consisting of: G₄A, and (G₄A)₃, (G₄A)₂G₃AG₄S, G₄SDAA, (G₄A)₂G₄S, G₄SDAA, (G₄S)₄, and G₄AG₃AG₄S.

18. The fusion protein of any one of claims 15-17, wherein the fusion protein
(a) has an amino acid sequence of SEQ ID NO: 6, or a variant having up to 10 amino acid substitutions, additions, or deletions; or
(b) has an amino acid sequence of SEQ ID NO: 10, or a variant having up to 10 amino acid substitutions, additions, or deletions.

19. The fusion protein of any one of claims claim 15-17, wherein the fusion protein:
(a) has an amino acid sequence with at least 85% sequence identity to SEQ ID NO: 6; or
(b) has an amino acid sequence with at least 85% sequence identity to SEQ ID NO: 10.

20. A fusion protein comprising the structure, from N-terminus to C-terminus:
D1-L1-D2-L2-D3, wherein:
D1 comprises an integrin recognition domain, such as cyclo(RGD)₄;
L1 comprises a linker or is absent;
D2 is a VHH, such as a single domain antibody,
L2 is a linker or is absent; and
D3 is a FH fragment, such FH1-5.

21. The fusion protein of claim 20, wherein the fusion protein has a C-terminal His tag.

22. The fusion protein of claim 20 or 21, wherein L1 and L2 comprise the same amino acid sequence.

23. The fusion protein of any one of claims 20-22, wherein L1 and L2 comprise different amino acid sequences.

24. The fusion protein of claim 22 or 23, wherein L1 and/or L2 are selected from the group consisting of: G₄A, (G₄A)₃, (G₄A)₂G₃AG₄S, G₄SDAA, (G₄A)₂G₄S, G₄AG₃AG₄S, GGGGAGGGGAGGGGS, GGGGSGGGGSGGGGS, G₄S, (G₄S)₂, (G₄S)₃, (G₄S)₄, (G₄S)₅, (G₄S)₆, (EAAAK)₃, PAPAP, G₄SPAPAP, PAPAPG₄S, GSTSGKSSEGKG, (GGGDS)₂, (GGGES)₂, GGGDSGGGGS, GGGASGGGGS, GGGESGGGGS, ASTKGP, ASTKGPSVFPLAP, G₃P, G₇P, PAPNLLGGP, G₆, G₁₂, APELPGGP, SEPQPQPG, (G₃S2)₃, GGGGGGGGGSGGGS, GGGGSGGGGGGGGGS, (GGSSS)₃, (GS₄)₃, G₄A(G₄S)₂, G₄SG₄AG₄S, G₃AS(G₄S)₂, G₄SG₃ASG₄S, G₄SAG₃SG₄S, (G₄S)₂AG₃S, G₄SAG₃SAG₃S, G₄D(G₄S)₂, G₄SG₄DG₄S, (G₄D)₂G₄S, G₄E(G₄S)₂, G₄SG₄EG₄S, (G₄E)₂G₄S, and G₄SDA.

25. The fusion protein of claim 24, wherein L1 and/or L2 are selected from the group consisting of: G₄A, (G₄A)₃, (G₄A)₂G₃AG₄S, G₄SDAA, (G₄A)₂G₄S, G₄SDAA, (G₄S)₄, and G₄AG₃AG₄S.

26. The fusion protein of claim 18 or 19, wherein the fusion protein
(a) has an amino acid sequence of SEQ ID NO: 2, or a variant having up to 10 amino acid substitutions, additions, or deletions; or
(b) has an amino acid sequence of SEQ ID NO: 3, or a variant having up to 10 amino acid substitutions, additions, or deletions.

27. The fusion protein of any one of claims 20-26, wherein the fusion protein
(a) has an amino acid sequence with at least 85% sequence identity to SEQ ID NO: 2; or
(b) has an amino acid sequence with at least 85% sequence identity to SEQ ID NO: 3.

28. A fusion protein comprising the structure, from N-terminus to C-terminus:
D1-D2 or D2-D1, wherein:
D1 is a VHH, such as a single domain antibody, and
D2 is a FH fragment, such as FH1-5.

29. The fusion protein of claim 28, wherein the fusion protein has a C-terminal His tag.

30. The fusion protein of claim 28 or 29, wherein the fusion protein
(a) has an amino acid sequence of SEQ ID NO: 1, or a variant having up to 10 amino acid substitutions, additions, or deletions; or
(b) has an amino acid sequence of SEQ ID NO: 7, or a variant having up to 10 amino acid substitutions, additions, or deletions.

31. The fusion protein of claim 28 or 29, wherein the fusion protein
(a) has an amino acid sequence with at least 85% sequence identity to SEQ ID NO: 1; or
(b) has an amino acid sequence with at least 85% sequence identity to SEQ ID NO: 7.

32. The fusion protein of claim 28 or 29, wherein the fusion protein
(a) has an amino acid sequence of SEQ ID NO: 11, or a variant having up to 10 amino acid substitutions, additions, or deletions; or
(b) has an amino acid sequence of SEQ ID NO: 12, or a variant having up to 10 amino acid substitutions, additions, or deletions.

33. The fusion protein of claim 28 or 29, wherein the fusion protein
(a) has an amino acid sequence with at least 85% sequence identity to SEQ ID NO: 11; or
(b) has an amino acid sequence with at least 85% sequence identity to SEQ ID NO: 12.

34. The fusion protein of any one of claims 1-33, wherein the fusion protein has an increased intrarenal residence time relative to the fusion protein lacking the VHH domain.

35. A pharmaceutical composition comprising the fusion protein of any one of claims 1-34 and a pharmaceutically acceptable carrier.

36. A polynucleotide encoding the fusion protein of any one of claims 1-35.

37. A vector comprising the polynucleotide of claim 36.

38. A host cell comprising the polynucleotide of claim 36 or the vector of claim 37.

39. A method of producing the fusion protein of any one of claims 1-34, comprising the steps of culturing one or more host cells comprising one or more nucleic acid molecules capable of expressing the fusion protein under conditions suitable for expression of the fusion protein.

40. The method of claim 39, wherein the method further comprises the step of obtaining the fusion protein from the cell culture or culture medium.

41. A method of treating a disease mediated by complement alternative pathway activation or dysregulation comprising administering an effective amount of a composition comprising the fusion protein of any one of claims 1-34, the pharmaceutical composition of claim 35, the polynucleotide of claim 36, the vector of claim 37, or the host cell of claim 38 to a subject in need thereof.

42. The method of claim 41, wherein the fusion protein is formulated as a pharmaceutical composition with at least one pharmaceutically acceptable carrier.

43. The method of claim 42, wherein the composition is lyophilized.

44. The method of claim 43, wherein the composition is rehydrated prior to administration.

45. The method of claim 42, wherein the at least one pharmaceutically acceptable carrier is saline.

46. The method of any one of claims 41-45, wherein the composition is formulated for daily, weekly, or monthly administration.

47. The method of any one of claims 41-46, wherein the composition is formulated for intravenous, subcutaneous, intramuscular, oral, nasal, sublingual, intrathecal, and intradermal administration.

48. The method of any one of claims 41-47, wherein the composition is formulated for administration at a dosage of between about 0.1 mg/kg to about 150 mg/kg.

49. The method of any one of claims 41-48, wherein the composition is formulated for administration in combination with an additional therapeutic agent.

50. The method of any one of claims 41-49, wherein the disease is a kidney disorder, such as focal segmental glomerulosclerosis (FSGS), IgA nephropathy, minimal change disease (MCD), diabetic nephropathy, Alport syndrome, lupus nephritis, membranous nephropathy, acute kidney injury, Goodpasture syndrome, nephrotic syndrome, chronic proteinuria, chronic kidney disease, C3 glomerulopathy (C3G), dense deposit disease, glomerulonephritis, membranoproliferative glomerulonephritis, polycystic kidney disease, hypertensive nephropathy, nephrosclerosis, atypical hemolytic uremic syndrome (aHUS), ischemia reperfusion injury, or rejection of a transplanted organ, such as a kidney.

51. The method of any one of claims 41-50, wherein the disease is FSGS.

52. The method of any one of claims 41-51, wherein the subject is a mammal.

53. The method of claim 52, wherein the mammal is a human.

54. A kit comprising a composition selected from the fusion protein of any one of claims 1-34, the pharmaceutical composition of claim 35, the polynucleotide of claim 36, the vector of claim 37, or the host cell of claim 38.

55. The kit of claim 54, further comprising instructions for administering an effective amount of the composition to a subject in need thereof.

56. The use of a composition comprising a fusion protein of any one of claims 1-34 for the manufacture of a medicament for a disease mediated by complement alternative pathway activation or dysregulation.

57. The use of claim 56, wherein the disease is a kidney disorder, such as FSGS, IgA nephropathy, MCD, diabetic nephropathy, Alport syndrome, lupus nephritis, membranous nephropathy, acute kidney injury, Goodpasture syndrome, nephrotic syndrome, chronic proteinuria, chronic kidney disease, C3G, dense deposit disease, glomerulonephritis, membranoproliferative glomerulonephritis, polycystic kidney disease, hypertensive nephropathy, nephrosclerosis, aHUS, ischemia reperfusion injury, or rejection of a transplanted organ, such as a kidney.
